SEQUENCE LISTING (68 sequences)

SEQ ID NO: 1 - protein, 8 aa, Artificial Sequence
  Description of Artificial Sequence Synthetic peptide
GFSLTDYG

SEQ ID NO: 2 - protein, 7 aa, Artificial Sequence
  Description of Artificial Sequence Synthetic peptide
IWGGGST

SEQ ID NO: 3 - protein, 14 aa, Artificial Sequence
  Description of Artificial Sequence Synthetic peptide
AKEKRRGYYYAMDY

SEQ ID NO: 4 - protein, 7 aa, Artificial Sequence
  Description of Artificial Sequence Synthetic peptide
SSVSSSY

SEQ ID NO: 5 - protein, 3 aa, Artificial Sequence
  Description of Artificial Sequence Synthetic peptide
STS

SEQ ID NO: 6 - protein, 9 aa, Artificial Sequence
  Description of Artificial Sequence Synthetic peptide
QQYSGYPLT

SEQ ID NO: 7 - DNA, 360 bp, Artificial Sequence
  Description of Artificial Sequence Synthetic polynucleotide
caggtgcagctgaaggagtcaggacctggcctggtggcgccctcacagagcctgtccatcacatgcactgtctcagggttctcattaaccgactatggtgtaaggtggattcgccagcctccaggaaagggtctggagtggctgggagtaatatggggtggtggaagcacatactataattcagctctcaaatccagactgagcatcagcaaggacaactccaagagccaagttttcttaaaaatgaacagtctgcaaactgatgacacagccatgtactactgtgccaaagagaaacggagggggtattactatgctatggactactggggtcaaggaacctcagtcaccgtctcctca

SEQ ID NO: 8 - DNA, 327 bp, Artificial Sequence
  Description of Artificial Sequence Synthetic polynucleotide
ggagaaaatgtgctcacccagtctccagcaatcatgtctgcatctccaggggaaaaggtcaccatgacctgcagggccagctcaagtgtaagttccagttacttgcactggtaccagcagaagtcaggtgcctcccccaaactctggatttatagcacatccaacttggcttctggagtccctgctcgcttcagtggcagtgggtctgggacctcttactctctcacaatcagcagtgtggaggctgaagatgctgccacttattactgccagcagtacagtggttacccactcacgttcggaggggggaccaagctggaaataaaa

SEQ ID NO: 9 - protein, 8 aa, Artificial Sequence
  Description of Artificial Sequence Synthetic peptide
GYSFTGYY

SEQ ID NO: 10 - protein, 8 aa, Artificial Sequence
  Description of Artificial Sequence Synthetic peptide
INPYNGAT

SEQ ID NO: 11 - protein, 8 aa, Artificial Sequence
  Description of Artificial Sequence Synthetic peptide
ARLDRGDY

SEQ ID NO: 12 - protein, 6 aa, Artificial Sequence
  Description of Artificial Sequence Synthetic peptide
ENVVTY

SEQ ID NO: 13 - protein, 3 aa, Artificial Sequence
  Description of Artificial Sequence Synthetic peptide
GAS

SEQ ID NO: 14 - protein, 9 aa, Artificial Sequence
  Description of Artificial Sequence Synthetic peptide
GQGYSYPYT

SEQ ID NO: 15 - DNA, 345 bp, Artificial Sequence
  Description of Artificial Sequence Synthetic polynucleotide
gaggtacagctgcagcagtctggacctgagctggtgaagcctggggcttcagtgaagatatcctgcaaggcttctggttactcattcactggctactacatgcactgggtgaagcaaagccatgtaaagagccttgagtggattggacgtattaatccttacaatggtgctactagctacaaccagaatttcaaggacaaggccagcttgactgtagataagtcctccagcacagcctacatggagctccacagcctgacatctgaggactctgcagtctattactgtgcaagactagaccggggggactactggggtcaaggaacctcagtcaccgtctcctca

SEQ ID NO: 16 - DNA, 327 bp, Artificial Sequence
  Description of Artificial Sequence Synthetic polynucleotide
aacattgtaatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggacaaagttggaaataaaacgtacg

SEQ ID NO: 17 - protein, 8 aa, Artificial Sequence
  Description of Artificial Sequence Synthetic peptide
GYTFTRYW

SEQ ID NO: 18 - protein, 8 aa, Artificial Sequence
  Description of Artificial Sequence Synthetic peptide
IYPGNSDT

SEQ ID NO: 19 - protein, 14 aa, Artificial Sequence
  Description of Artificial Sequence Synthetic peptide
ARGEEIGVRRWFAY

SEQ ID NO: 20 - protein, 6 aa, Artificial Sequence
  Description of Artificial Sequence Synthetic peptide
QSISNY

SEQ ID NO: 21 - protein, 3 aa, Artificial Sequence
  Description of Artificial Sequence Synthetic peptide
YAS

SEQ ID NO: 22 - protein, 9 aa, Artificial Sequence
  Description of Artificial Sequence Synthetic peptide
QQSNSWPLT

SEQ ID NO: 23 - DNA, 363 bp, Artificial Sequence
  Description of Artificial Sequence Synthetic polynucleotide
caggtccaactgcagcagtcaggagctgaactggtcaagactggggcctcagtgaagatgtcctgcaaggcttctggctacacctttaccagatactggatgcactgggtaaaacagaggcctggacaggctctggaatggattggcgctatttatcctggaaatagtgatactagctactaccagaagttcaagggcaaggccaaactgactgcagtcacatctgccagcactgcctacatggagctcagcagcctgacatctgaggactctgccgtctattactgtgcaagaggggaggaaataggggtacgacgctggtttgcttactggggccaagggactctggtcactgtctctgca

SEQ ID NO: 24 - DNA, 321 bp, Artificial Sequence
  Description of Artificial Sequence Synthetic polynucleotide
gatattgtgctaactcagtctccagccaccctgtctgtgactccaggagatagagtcagtctttcctgcagggccaggcaaagtattagcaactacctacactggtatcaacaaaaatcacatgagtctccaaggcttctcatcaagtatgcttcccagtccatctctggcatcccctccaggttcagtggcagtggatcagggacagatttcactctcagtatcaacagtgtggagactgaagattttggaatgtatttctgtcaacagagtaacagctggccgctcacgttcggtgctgggaccaagctggaaataaaa

SEQ ID NO: 25 - protein, 6 aa, Artificial Sequence
  Description of Artificial Sequence Synthetic peptide
SGYYWG

SEQ ID NO: 26 - protein, 16 aa, Artificial Sequence
  Description of Artificial Sequence Synthetic peptide
SIYHSGSTYYNPSLKS

SEQ ID NO: 27 - protein, 8 aa, Artificial Sequence
  Description of Artificial Sequence Synthetic peptide
GKWSKFDY

SEQ ID NO: 28 - protein, 11 aa, Artificial Sequence
  Description of Artificial Sequence Synthetic peptide
QGDSLRSYYAS

SEQ ID NO: 29 - protein, 7 aa, Artificial Sequence
  Description of Artificial Sequence Synthetic peptide
GKNNRPS

SEQ ID NO: 30 - protein, 11 aa, Artificial Sequence
  Description of Artificial Sequence Synthetic peptide
NSRDSSGNHVV

SEQ ID NO: 31 - DNA, 351 bp, Artificial Sequence
  Description of Artificial Sequence Synthetic polynucleotide
caggtgcagctgcaggagtccggcccaggactggtgaagccttcggagaccctgtccctcacctgcgctgtctctggttactccatcagcagtggttactactggggctggattcggcagcccccagggaaggggctggagtggattgggagtatctatcatagtgggagcacctactacaacccgtccctcaagagtcgagtcaccatatcagtagacacgtccaagaaccagttctccctgaagctgagctctgtgaccgccgcagacacggccgtgtattactgtgcaagagggaagtggtcgaagtttgactattggggccaaggcaccctggtcaccgtctcttca

SEQ ID NO: 32 - DNA, 324 bp, Artificial Sequence
  Description of Artificial Sequence Synthetic polynucleotide
tcctctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggattccagaccgattctctggctccagctcaggaaacacagcttccttgaccatcactggggctcaggcggaagatgaggctgactattactgtaactcccgggacagcagtggtaaccatgtggtattcggcggagggaccaagctgaccgtccta

SEQ ID NO: 33 - protein, 51 aa, Homo sapiens
PAKPTTTPAPRPPTPAPTIASQPLSLRPEACRPAAGGAVHTRGLDFACDIY

SEQ ID NO: 34 - protein, 49 aa, Mus musculus
KVNSTTTKPVLRTPSPVHPTGTSQPQRPEDCRPRGSVKGTGLDFACDIY

SEQ ID NO: 35 - protein, 51 aa, Felis catus
PVKPTTTPAPRPPTQAPITTSQRVSLRPGTCQPSAGSTVEASGLDLSCDIY

SEQ ID NO: 36 - protein, 21 aa, Homo sapiens
IYIWAPLAGTCGVLLLSLVIT

SEQ ID NO: 37 - protein, 21 aa, Mus musculus
IWAPLAGICVALLLSLIITLI

SEQ ID NO: 38 - protein, 21 aa, Rattus norvegicus
IWAPLAGICAVLLLSLVITLI

SEQ ID NO: 39 - protein, 42 aa, Unknown
  Description of Unknown 4-1BB costimulatory signaling region
KRGRKKLLYIFKQPFMRPVQTTQEEDGCSCRFPEEEEGGCEL

SEQ ID NO: 40 - protein, 220 aa, Unknown
  Description of Unknown CD28 polypeptide
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 41 - protein, 112 aa, Unknown
  Description of Unknown CD3 zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 43 - protein, 98 aa, Homo sapiens
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 44 - protein, 96 aa, Homo sapiens
MCCTKSLLLAALMSVLLLHLCGESEAASNFDCCLGYTDRILHPKFIVGFTRQLANEGCDINAIIFHTKKKLSVCANPKQTWVKYIVRLLSKKVKNM

SEQ ID NO: 45 - protein, 95 aa, Homo sapiens
MCCTKSLLLAALMSVLLLHLCGESEASNFDCCLGYTDRILHPKFIVGFTRQLANEGCDINAIIFHTKKKLSVCANPKQTWVKYIVRLLSKKVKNM

SEQ ID NO: 46 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 47 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 48 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 49 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 50 - DNA, 1611 bp, Homo sapiens
catggccatgtgtcatcagcagctggtcatcagctggttcagcctggtgttcctggccagccccctggtggccatctgggagctgaagaaagacgtgtacgtggtggagctggactggtatcccgacgcccctggcgagatggtggtgctgacctgcgacacccccgaagaggacggcatcacctggaccctggaccagagcagcgaggtgctgggcagcggcaagaccctgaccatccaggtcaaagagttcggcgacgccggccagtacacctgccacaagggcggcgaagtgctgtcccacagcctgctgctgctgcacaagaaagaggatggcatctggtccaccgacatcctgaaggaccagaaagagcccaagaacaagaccttcctgcggtgcgaggccaagaactacagcggccggttcacctgttggtggctgaccaccatcagcaccgacctgaccttcagcgtgaagagcagccggggcagcagcgaccctcagggcgtgacctgcggagccgccaccctgagcgccgagagagtgcggggcgacaacaaagagtacgagtacagcgtcgagtgccaggaagatagcgcctgccctgccgccgaggaaagcctgcccatcgaggtgatggtggacgccgtgcacaagctgaagtacgagaactacacctccagctttttcatccgggacatcatcaagcccgacccccccaagaacctgcagctgaagcccctgaagaacagccggcaggtggaggtgtcctgggagtaccctgacacctggtccaccccccacagctacttcagcctgaccttctgtgtgcaggtgcagggcaagagcaagcgggagaagaaagaccgggtgttcaccgacaagaccagcgccaccgtgatctgccggaagaacgccagcatcagcgtgcgggcccaggaccggtactacagcagctcctggtccgagtgggccagcgtgccctgcagcggcggagggggcggaggaagccggaacctgcccgtggctacccccgaccccggcatgttcccctgcctgcaccacagccagaacctgctgcgggccgtgagcaacatgctgcagaaggcccggcagaccctggaattctacccctgcaccagcgaggaaatcgaccacgaggacatcaccaaggataagaccagcaccgtggaggcctgcctgcccctggaactgaccaagaacgagagctgtctgaactctcgggagacaagcttcatcaccaacggctcttgcctggccagcagaaagaccagcttcatgatggccctgtgcctgagcagcatctacgaggacctgaagatgtaccaggtggagttcaagaccatgaacgccaagctgctgatggaccccaagcggcagatcttcctggatcagaacatgctggccgtgatcgacgagctgatgcaggccctgaacttcaacagcgagacagtgccccagaagtccagcctggaagagcccgacttctacaagaccaagatcaagctgtgcatcctcctgcatgccttccggatccgggccgtgaccatcgaccgggtgatgagctacctgaacgccagctgatgagc

SEQ ID NO: 51 - protein, 133 aa, Homo sapiens [sequence omitted]

SEQ ID NO: 52 - protein, 113 aa, Homo sapiens
WVNVISDLKKIEDLIQSMHIDATLYTESDVHPSCKVTAMKCFLLELQVISLESGDASIHDTVENLIILANNSLSSNGNVTESGCKECEELEKKNIKEFLQSFVHIVQMFINTS

SEQ ID NO: 53 - protein, 193 aa, Homo sapiens
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 54 - protein, 133 aa, Homo sapiens [sequence omitted]

SEQ ID NO: 55 - protein, 120 aa, Homo sapiens
MKVSEAALSLLVLILIITSASRSQPKVPEWVNTPSTCCLKYYEKVLPRRLVVGYRKALNCHLPAIIFVTKRNREVCTNPNDDWVQEYIKDPNLPLLPTRNLSTVKIITAKNGQPQLLNSQ

SEQ ID NO: 56 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 57 - DNA, 29 bp, Homo sapiens
ggaggaaaaactgtttcatacagaaggcg

SEQ ID NO: 58 - DNA, 9 bp, Artificial Sequence
  Description of Artificial Sequence Synthetic oligonucleotide
aggaaaaac

SEQ ID NO: 59 - DNA, 48 bp, Unknown
  Description of Unknown IgG1 heavy chain hinge sequence
ctcgagcccaaatcttgtgacaaaactcacacatgcccaccgtgcccg

SEQ ID NO: 60 - DNA, 81 bp, Unknown
  Description of Unknown CD28 transmembrane region
ttttgggtgctggtggtggttggtggagtcctggcttgctatagcttgctagtaacagtggcctttattattttctgggtg

SEQ ID NO: 61 - DNA, 126 bp, Unknown
  Description of Unknown 4-1BB co-stimulatory signaling region
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 62 - DNA, 123 bp, Unknown
  Description of Unknown CD28 co-stimulatory signaling region
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 63 - DNA, 339 bp, Unknown
  Description of Unknown CD3 zeta signaling region
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 64 - DNA, 105 bp, Unknown
  Description of Unknown ICOS costimulatory signaling region
acaaaaaagaagtattcatccagtgtgcacgaccctaacggtgaatacatgttcatgagagcagtgaacacagccaaaaaatccagactcacagatgtgacccta

SEQ ID NO: 65 - DNA, 108 bp, Unknown
  Description of Unknown OX40 costimulatory signaling region
agggaccagaggctgccccccgatgcccacaagccccctgggggaggcagtttccggacccccatccaagaggagcaggccgacgcccactccaccctggccaagatc

SEQ ID NO: 66 - protein, 12 aa, Artificial Sequence
  Description of Artificial Sequence Synthetic peptide
  MISC_FEATURE (1)..(12): This sequence may encompass 1-6 repeating “Gly Ser” units
GSGSGSGSGSGS

SEQ ID NO: 67 - protein, 15 aa, Artificial Sequence
  Description of Artificial Sequence Synthetic peptide
GGGGSGGGGSGGGGS

SEQ ID NO: 68 - protein, 5 aa, Artificial Sequence
  Description of Artificial Sequence Synthetic peptide
GGGGS

(19) United States
(12) Patent Application Publication
(10) Pub. No.: US 2018/0291089 A1
(43) Pub. Date: Oct. 11, 2018

(54) SECRETORY TNT CAR CELL IMMUNOTHERAPY

(71) Applicant: University of Southern California, Los Angeles, CA (US)

(72) Inventors: Alan L. Epstein, Pasadena, CA (US); Harvey Kaslow, Los Angeles, CA (US); Peisheng Hu, Covina, CA (US)

(21) Appl. No.: 15/570,747

(22) PCT Filed: Apr. 29, 2016
(86) PCT No.: PCT/US16/30248

§ 371 Date: Oct. 30, 2017

Related U.S. Application Data
(60) Provisional application No. 62/155,296, filed on Apr. 30, 2015.

(51) Int. Cl. C07K 16/18; C07K 14/705; C07K 14/725; C07K 14/47; A61K 45/06; A61K 38/17; A61K 39/395; A61K 38/20; A61K 38/19; C07K 14/54; C07K 14/535; C07K 14/55; C07K 14/52

(52) U.S. Cl. CPC C07K 16/18; A61K 2039/5158; C07K 14/70521; C07K 14/7051; C07K 14/70578; C07K 14/4748; A61K 45/06; A61K 38/1774; A61K 38/177; A61K 39/3955; A61K 38/208; A61K 38/193; C07K 14/5434; C07K 14/535; A61K 38/2013; C07K 14/55; C07K 14/5443; A61K 38/2086; C07K 14/54; A61K 38/20; C07K 14/70532; C07K 14/70575; A61K 38/195; C07K 14/521; C07K 2319/30; C07K 2317/53; C07K 2317/565; C07K 2317/56; C07K 2319/40; C07K 2319/33; C07K 2317/24; C07K 14/70517

(57) ABSTRACT

CAR cells targeting tumor necrosis therapy relevant antigens are described as a new method of cancer treatment. It is proposed that TNT CAR cells are safe and effective in patients and can be used to treat human tumors and cancer.

### CROSS-REFERENCE TO RELATED APPLICATION

This application claims priority under 35 U.S.C. § 119(e) to U.S. Provisional Application No. 62/155,296, filed Apr. 30, 2015, the content of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Apr. 29, 2016, is named 064189-7181_SL.txt and is 42,860 bytes in size.

### TECHNICAL FIELD

The present disclosure relates generally to the field of human immunology, specifically cancer immunotherapy.

### BACKGROUND

The following discussion of the background of the invention is merely provided to aid art to the present invention.

A unique approach to cancer imaging and therapy utilizing necrotic cells as a target for the selective binding of monoclonal antibodies (MAbs) has been developed by applicant's research group. (Epstein, A. L. et al. (1988) Cancer Res. 48:5842-5848; Chen, F. M. et al. (1991) J Natl Cancer Inst. 83:200-204; Epstein, A. L. et al. (1991) Antibody, Immunoconj & Radiopharm. 4:151-161; Miller, G. K. et al. (1993) Hybridoma 12:689-698; Hornick, J. L. et al. (1998) Cancer Biother & Radiopharm 13:255-268). Tumor Necrosis Therapy (TNT) represents a radical departure from other methods that employ MAbs to bind to tumor-associated cell surface antigens. In contrast, TNT MAbs recognize intracellular antigens which are revealed and retained by the cell ghost when normal or tumor cells die or become compromised—for example, by hypoxia, necrosis, apoptosis, or cytotoxic reagents and/or processes. Thus, TNT MAbs localize in malignant tumors due to the presence of permeable, degenerating cells not found in normal tissues.

### SUMMARY

Aspects of the disclosure relate to a chimeric antigen receptor (CAR) comprising, or alternatively consisting essentially of, or yet further consisting of: (a) an antigen binding domain of a TNT antibody; (b) a hinge domain; (c) a transmembrane domain; and (d) an intracellular domain. Further aspects of the disclosure relate to a chimeric antigen receptor (CAR) comprising, or alternatively consisting essentially of, or yet further consisting of: (a) an antigen binding domain of a TNT antibody; (b) a CD8 α hinge domain; (c) a CD8 α transmembrane domain; (d) a CD28 costimulatory signaling region and/or a 4-1BB costimulatory signaling region; and (e) a CD3 zeta signaling domain.

In certain embodiments, the antigen binding domain of the TNT antibody comprises, or alternatively consists essentially thereof, or further consists of a TNT heavy chain variable region and a TNT light chain variable region.

In some embodiments, the TNT heavy chain variable region comprises, or alternatively consists essentially thereof, or further consists of a CDR region comprising any one of SEQ ID NOs: 1-3, 9-11, 17-19, 25-27 or an equivalent of each thereof. In some embodiments, the TNT heavy chain variable region comprises, or alternatively consists essentially thereof, or further consists of an amino acid sequence encoded by any one of SEQ ID NOs: 7, 15, 23, 31 or an equivalent of each thereof.

In some embodiments, the TNT light chain variable region comprises a CDR region comprising, or alternatively consisting essentially of, or yet further consisting of any one of SEQ ID NOs: 4-6, 12-14, 28-30 or an equivalent of each thereof. In some embodiments, the TNT light chain variable region comprises, or alternatively consists essentially thereof, or further consists of an amino acid sequence encoded by any one of SEQ ID NOs: 8, 16, 24, 32 or an equivalent of each thereof.

In certain embodiments, the CAR further comprises, or alternatively further consists essentially of, or yet further consists of, a linker polypeptide located between the TNT heavy chain variable region and the TNT light chain variable region. In certain embodiments, the linker is a glycine-serine linker. In further embodiments, the linker polypeptide comprises, or alternatively consists essentially thereof, or further consists of the sequence (glycine-serine)n wherein n is an integer from 1 to 6 (SEQ ID NO: 66).

In certain embodiments, the CAR further comprises, or alternatively further consists essentially of, or yet further consists of, a detectable marker and/or a purification marker attached to the CAR.

Additional aspects of the disclosure relate to an isolated nucleic acid sequence encoding a CAR, as described above, or its complement, or an equivalent of each thereof.

In certain embodiments, the isolated nucleic acid sequence further comprises, or further consists essentially of, or yet further consists of, a Kozak consensus sequence located upstream of the polynucleotide encoding the antigen binding domain of the TNT antibody.

In certain aspects, the isolated nucleic acid further comprises, or alternatively consists essentially thereof, or further consists of a polynucleotide encoding an antibiotic resistance polypeptide operatively coupled to the isolated nucleic acid.

Aspects of the disclosure relate to a vector comprising one or more of the isolated nucleic acids described above. In certain embodiments, the vector is a plasmid or a viral vector selected from the group of a retroviral vector, a lentiviral vector, an adenoviral vector, and an adeno-associated viral vector. The isolated nucleic acids and vectors containing them are useful to prepare the CARs as described herein.

Further aspects of the disclosure relate to an isolated cell comprising, or alternatively consisting essentially thereof, or further consisting of one or more of the above described compositions: a TNT CAR, an isolated nucleic acid encoding a CAR or its complement, or a vector containing the isolated nucleic acid. In certain embodiments, the isolated cell may be a prokaryotic cell such as a bacteria cell, e.g., an E. coli, or a eukaryotic cell. In some embodiments the isolated eukaryotic cell is selected from an animal cell, a mammalian cell, a bovine cell, a feline cell, a canine cell, a murine cell, an equine cell or a human cell. In further embodiments, the isolated cell is the cell is a T-cell, a B cell, or a NK cell, from any of the species as disclosed herein.

In certain embodiments, the isolated cell further comprises, or alternatively consists essentially of, or yet further consists of, an isolated nucleic acid comprising, or alternatively consisting essentially of, or yet further consisting of an NFAT regulatory polynucleotide operatively linked to a polynucleotide encoding an immunoregulatory molecule. Non-limiting examples of an isolated cell is a prokaryotic cell such as a bacteria cell, e.g., an E coli, or a eukaryotic cell. In some embodiments the isolated eukaryotic cell is selected from an animal cell, a mammalian cell, a bovine cell, a feline cell, a canine cell, a murine cell, an equine cell or a human cell. In further embodiments, the isolated cell is the cell is a T-cell, a B cell, or a NK cell from any of the species as disclosed herein.

In certain embodiments, the isolated nucleic acid further comprises, or alternatively consists essentially thereof, or yet further consists of a polynucleotide encoding an antibiotic resistance gene. In certain embodiments, the immunoregulatory molecule is one or more molecule selected from the group consisting of B7.1, CCL19, CCL21, CD40L, CD137L, GITRL, GM-CSF, IL-12, IL-2, low-toxicity IL-2, IL-15, IL-18, IL-21, LEC, and OX40L.

Aspects of the disclosure relate to a composition comprising, or alternatively consisting essentially of, or further consisting of one or more of the above described compositions, e.g., a CAR, an isolated nucleic acid, a cell, or a vector and a carrier.

In certain embodiments, the composition further comprises, or alternatively consists essentially of, or yet further consists of, an immunoregulatory molecule and/or an isolated nucleic acid comprising an NFAT regulatory polynucleotide operatively linked to a polynucleotide encoding an immunoregulatory molecule. In certain embodiments, the immunoregulatory molecule is one or more molecule selected from the group consisting of B7.1, CCL19, CCL21, CD40L, CD137L, GITRL, GM-CSF, IL-12, IL-2, low-toxicity IL-2, IL-15, IL-18, IL-21, LEC, and OX40L.

Aspects of the disclosure relate to an isolated complex comprising a CAR or a cell comprising the CAR bound to a TNT relevant antigen or a fragment thereof, and/or a cell expressing TNT relevant antigen. In one aspect, the antigen binding domain is expressed on the surface of the cell. In another aspect, the TNT relevant antigen is expressed in a tumor, e.g., the necrotic tissue of a tumor. A non-limiting example of a tumor is a solid tumor. A non-limiting example of a solid tumor is a tumor comprising a colon cancer cell. Other solid tumors are disclosed herein. In one aspect the cell containing or expressing the TNT CAR is a NK cell, a B cell, or a T cell. The tumors or cells can be from any animal, e.g., mammalian such as a human cell.

Some aspects of the disclosure relate to a method of producing a TNT CAR expressing cell, the method comprising, or alternatively consisting essentially thereof, or yet further consisting of transducing an isolated cell with a nucleic acid sequence encoding a CAR as described herein.

In a further aspect, the method further comprises selecting and isolating the cell expressing the CAR. In a further aspect, the cell is a eukaryotic cell such as a mammalian cell, e.g., a human cell such as a NK cell, a B cell, or T cell. The cells can be transduced using the viral vectors as described herein or alternatively using technology described in Riet et al. (2013) Meth. Mol. Biol. 969:187-201 entitled “Nonviral RNA transfection to transiently modify T cell with chimeric antigen receptors for adoptive therapy.”

In certain embodiments, the method further comprises, or alternatively consists essentially of, or yet further consists of transducing the cell with an isolated nucleic acid comprising, or alternatively consisting essentially of, or yet further consisting of an NFAT regulatory polynucleotide operatively linked to a polynucleotide encoding an immunoregulatory molecule. The cells can be transduced using the viral vectors as described herein or alternatively using technology described in Riet et al. (2013) Meth. Mol. Biol. 969:187-201 entitled “Nonviral RNA transfection to transiently modify T cell with chimeric antigen receptors for adoptive therapy.”

In certain embodiments, the immunoregulatory molecule is one or more selected from the group consisting of B7.1, CCL19, CCL21, CD40L, CD137L, GITRL, GM-CSF, IL-12, IL-2, low-toxicity IL-2, IL-15, IL-18, IL-21, LEC, and OX40L.

In certain embodiments, the method of producing a TNT CAR expressing cell further comprises, or alternatively consists essentially of, or yet further consists of activating and expanding the population of TNT CAR expressing cells. Certain aspects of the present disclosure relate to an isolated, activated population of cells comprising, or alternatively consisting essentially of, or yet further consisting of a TNT CAR. In certain embodiments, the cells are one or more of NK cells, B cells, or T cells.

Aspects of the disclosure relate to a method of inhibiting the growth of a tumor expressing a TNT relevant antigen, by contacting the tumor with an effective amount of the isolated cells or compositions disclosed above. The contacting can be in vitro or in vivo. When the contacting is in vitro, the method can be used to test personalized therapy against a patient's tumor or to assay for combination therapies. When the contacting is in vivo, the method is useful to inhibit the growth of the tumor in a subject in need thereof, such as a human patient suffering from cancer and the patient receives an effective amount of the cells. In certain embodiments, the tumor is a solid tumor. In certain embodiments, the cancer/tumor targeted is a solid tumor or a cancer affecting the blood and/or bone marrow. In certain embodiments the isolated cells are autologous to the subject being treated. In another aspect, the method further comprises, or consists essentially of, or yet further consists of, administering to the subject an effective amount of a cytoreductive therapy. In a further aspect, the method further comprises the steps of isolating the cells to be administered to the subject, transducing the cells with an effective amount of an isolated nucleic acid encoding a CAR as described herein, culturing the cells to obtain a population of CAR encoding cells, that are optionally expanded and activated and then administering the cells to the patient.

Also disclosed herein are kits comprising one or more of the above noted compositions and instructions for their use in the methods as disclosed herein.

### BRIEF DESCRIPTION OF THE DRAWINGS

FIG. 1 is a schematic diagram showing an approach to CAR T-cell formation in which the T-cell synapse is replaced by an antibody single chain construct to bypass HLA dependent T-cell activation. Linker sequence disclosed as SEQ ID NO: 67.

FIGS. 2A-2D show characteristics of TNT antibody binding to necrotic regions of tumors. FIG. 2A shows H & E stained human tumor with viable cords, newly necrotic areas, and old necrosis typical of all tumors. The ability of TNT to bind to tumors of both mice and man are shown in FIGS. 2B-2D. FIG. 2B shows the uptake of radiolabeled TNT antibodies in a subcutaneous Colon 26 tumor in a BALB/c mouse. FIG. 2C shows immunoscintigraphic image of radiolabeled chTNT-3 in the lungs of a patient with bronchioalveolar carcinoma composed of small tumor nodules in both the right and left lung fields. FIG. 2D shows immunoscintigraphic images of a patient injected with I-131 radiolabeled chTNT-3 with an apical posterior lung carcinoma mass. Images taken over time show initial imaging of blood pool which decreases in intensity over time while the image of the tumor remains over a ten day period demonstrating specificity to tumor. In addition, this patient had a purulent inflammatory lesion in the left lower abdominal area which did not image due to the finding that PMNs have a highly heterochromatic nucleus which hides the TNT antigen preventing TNT antibodies from binding. Bladder shows accumulation of metabolized I-131 radiolabel which is eliminated upon emptying.

FIGS. 3A-3B show autoradiographic studies demonstrating binding of radiolabeled TNT antibodies to necrotic regions of tumors. FIG. 3A is an H & E stained section of the human ME-180 cervical tumor heterotransplanted in a nude mouse. Lightly stained areas are necrotic and more darkly stained areas are viable tumor. FIG. 3B shows a serial section stained by macroscopic autoradiography. Darkly stained areas show deposition of radiolabeled antibody administered 48 hours previously. In this study, antibody is seen to stain only necrotic areas. An example of antibody binding to a necrotic region is shown by the red arrows while the yellow arrows mark viable zones unlabeled by antibody.

FIG. 4 shows LEC induced immune cell infiltration in Colon 26 murine tumor model. In these studies, tumor bearing mice were treated on days 7-11 after tumor implantation with either chTNT-3 alone (control) or LEC/chTNT-3 and sacrificed 3 days later. The top three panels were taken from control treated mice and the bottom three panels were obtained from LEC/chTNT-3 treated mice. Compared to control, the targeted LEC produced a massive infiltration of both PMNs and dendritic cells generating an effective immune response shown in the bottom right panel in which lymphoid infiltrates are seen surrounding newly generated necrotic areas and concomitant tumor vessel fibrosis and clotting.

FIG. 5 shows chemotaxic activity of LEC/chTNT-3. THP-1 human monocytic leukemia cells were used in a chemotaxis chamber to determine the biologic activity of the LEC/chTNT-3, free LEC, and chTNT-3 (negative control).

FIGS. 6A-6B show schematic diagrams of inducible IL-12 and LEC genes.

FIG. 7 shows a schematic diagram of inducible bicistronic IL-12 and LEC genes.

FIG. 8A-8B shows a schematic diagrams of (in FIG. 8A) NFAT protein binding motif and inducible NFAT genes (SEQ ID NO: 57). TSS, Transcription Start Site and (in FIG. 8B) inducible bicistronic IL-12 and LEC genes.

FIG. 9 shows NK-92MI cells that were transduced with lentivirus containing the inducible NFAT-ZsGreen1 gene. ZsGreen1 is a modified GFP developed by Clontech Laboratories (Mountain View, Calif.). Transduced cells were cultured in complete RPMI for two weeks prior to being stimulated with 50 fmol rmIL-12. After 16 hours of stimulation, cells were assessed for ZsGreen1 expression.

FIG. 10 shows flow cytometry results for Jurkat cells that were transduced either with lentivirus containing the inducible NFAT-ZsGreen1 gene alone or in addition to a CD19 CAR. CAR-NFAT cells were incubated overnight with target CD19+ Raji cells at 1:1 CAR-to-target cell ratio. After 16 hours of incubation, cells were assessed for induced ZsGreen1 expression via flow cytometry.

FIG. 11 shows induced IL-12 secretion for Jurkat cells transduced with lentiviruses containing TNT-CAR, either alone or in addition to lentiviruses containing inducible NFAT-LEC or NFAT-IL12 genes. On a 24-well plate, transduced cells were incubated at 2×10⁵ cells in 500 μL complete RPMI either in wells coated with single-stranded calf thymus DNA (Sigma-Aldrich, St. Louis, Mo.) or with PMA (10 ng/mL) and ionomycin (500 ng/mL). After 16 hours of stimulation, cell supernatants were harvested and assayed for scmIL-12 via ELISA. scmIL-12, single-chain murine IL-12.

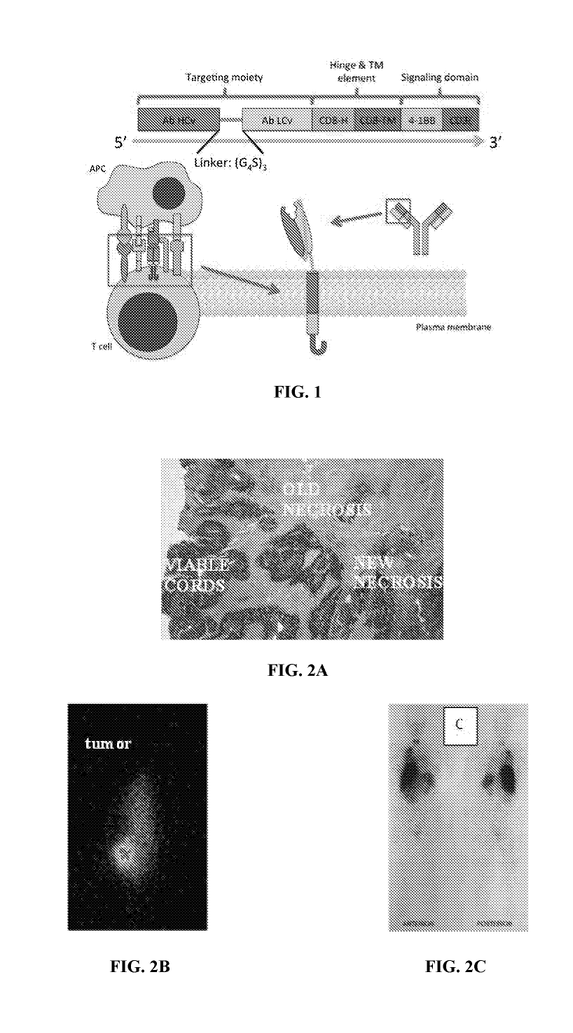

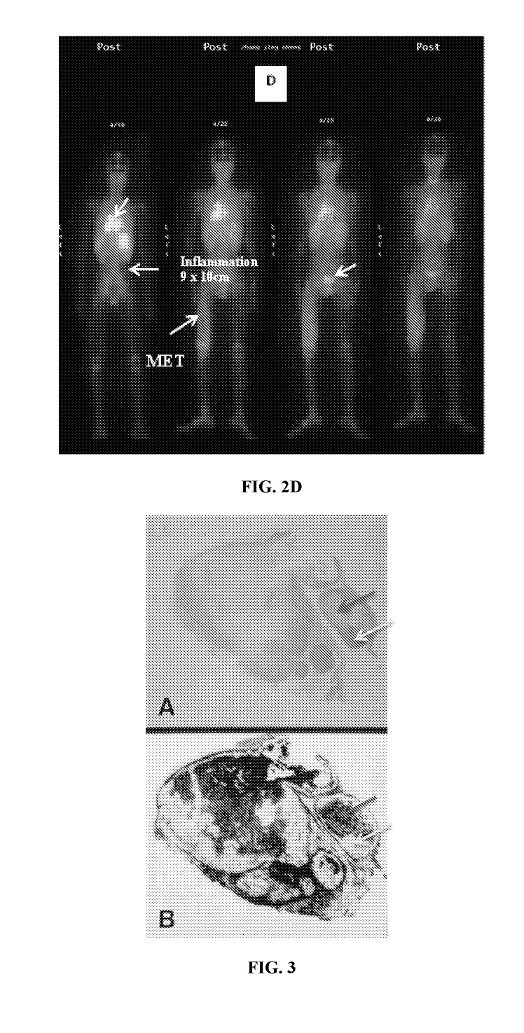

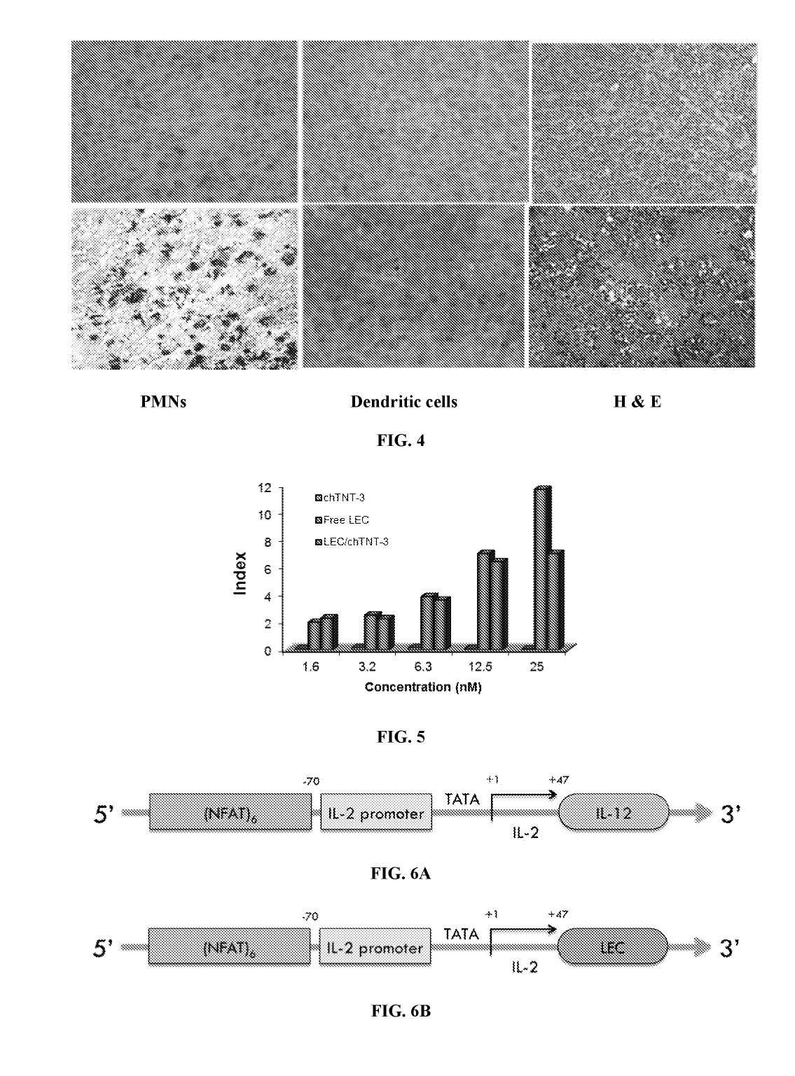

### DETAILED DESCRIPTION

It is to be understood that the present disclosure is not limited to particular aspects described, as such may, of course, vary. It is also to be understood that the terminology used herein is for the purpose of describing particular aspects only, and is not intended to be limiting, since the scope of the present disclosure will be limited only by the appended claims.

Unless defined otherwise, all technical and scientific terms used herein have the same meanings as commonly understood by one of ordinary skill in the art to which this technology belongs. Although any methods and materials similar or equivalent to those described herein can be used in the practice or testing of the present technology, the preferred methods, devices and materials are now described. All technical and patent publications cited herein are incorporated herein by reference in their entirety. Nothing herein is to be construed as an admission that the present technology is not entitled to antedate such disclosure by virtue of prior invention.

The practice of the present technology will employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

It is to be inferred without explicit recitation and unless otherwise intended, that when the present technology relates to a polypeptide, protein, polynucleotide or antibody, an equivalent or a biologically equivalent of such is intended within the scope of the present technology.

### Definitions

As used in the specification and claims, the singular form “a”, “an”, and “the” include plural references unless the context clearly dictates otherwise. For example, the term “a cell” includes a plurality of cells, including mixtures thereof.

As used herein, the term “animal” refers to living multi-cellular vertebrate organisms, a category that includes, for example, mammals and birds. The term “mammal” includes both human and non-human mammals.

The terms “subject,” “host,” “individual,” and “patient” are as used interchangeably herein to refer to human and veterinary subjects, for example, humans, animals, non-human primates, dogs, cats, sheep, mice, horses, and cows. In some embodiments, the subject is a human.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴M⁻¹ greater or at least 10⁵M⁻¹ greater than a binding constant for other molecules in a biological sample). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

As used herein, the term “monoclonal antibody” refers to an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

In terms of antibody structure, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located (heavy chain regions labeled CDHR and light chain regions labeled CDLR). Thus, a CDHR3 is the CDR3 from the variable domain of the heavy chain of the antibody in which it is found, whereas a CDLR1 is the CDR1 from the variable domain of the light chain of the antibody in which it is found. A TNT antibody will have a specific V_{H} region and the V_{L} region sequence unique to the TNT relevant antigen, and thus specific CDR sequences. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

As used herein, the term “antigen binding domain” refers to any protein or polypeptide domain that can specifically bind to an antigen target.

As used herein, the term “TNT” when used to describe an antibody refers to an antibody that recognizes a tumor necrosis therapy (TNT) relevant antigen or a functional equivalent thereof. A “tumor necrosis therapy (TNT) relevant antigen” is an antigen where the whole antigen, epitope, or fragment thereof is retained by a dying cell and where the whole antigen, epitope, or fragment would not normally be exposed but for cell death. Non-limiting examples of such TNT relevant antigens are DNA or DNA/histone targets usually made accessible only after cell death; for instance, TNT-1 binds to a portion of histone H1. Other histone targets include but are not limited to H2A, H2B, H3, H4, H5 and/or other human or animal histones. Other TNT relevant antigens include, but are not limited to, single stranded DNA and heterochromatic DNA. TNT antibodies include, but are not limited to TNT-1, TNT-2, TNT-3, and NHS76; the CDRs of which are listed below or known in the art as described in U.S. Pat. No. 8,795,672; U.S. Pat. No. 8,545,838.

As used herein, the term TNT-1 refers to an antibody comprising an amino acid sequence with CDRs that share at least 70%, or alternatively at least 80% amino acid sequence identity, preferably 90% sequence identity, more preferably at least 95% sequence identity with any one of the CDRs, preferably at least one of the CDR3 regions, most preferably both of the CDR3 regions, disclosed below.

TNT-1 CDHR1, SEQ ID NO: 1:
GFSLTDYG
TNT-1 CDHR2, SEQ ID NO: 2:
IWGGGST
TNT-1 CDHR3, SEQ ID NO: 3:
AKEKRRGYYYAMDY
TNT-1 CDLR1, SEQ ID NO: 4:
SSVSSSY
TNT-1 CDLR2, SEQ ID NO: 5:
STS
TNT-1 CDLR3, SEQ ID NO: 6:
QQYSGYPLT
TNT-1 Heavy Chain Variable Region Sequence,
SEQ ID NO: 7:
CAGGTGCAGCTGAAGGAGTCAGGACCTGGCCTGGTGGCGCCCTCACAGA
GCCTGTCCATCACATGCACTGTCTCAGGGTTCTCATTAACCGACTATGG
TGTAAGGTGGATTCGCCAGCCTCCAGGAAAGGGTCTGGAGTGGCTGGGA
GTAATATGGGGTGGTGGAAGCACATACTATAATTCAGCTCTCAAATCCA
GACTGAGCATCAGCAAGGACAACTCCAAGAGCCAAGTTTTCTTAAAAAT
GAACAGTCTGCAAACTGATGACACAGCCATGTACTACTGTGCCAAAGAG
AAACGGAGGGGGTATTACTATGCTATGGACTACTGGGGTCAAGGAACCT
CAGTCACCGTCTCCTCA
TNT-1 Light Chain Variable Region Sequence,
SEQ ID NO: 8:
GGAGAAAATGTGCTCACCCAGTCTCCAGCAATCATGTCTGCATCTCCAG
GGGAAAAGGTCACCATGACCTGCAGGGCCAGCTCAAGTGTAAGTTCCAG
TTACTTGCACTGGTACCAGCAGAAGTCAGGTGCCTCCCCCAAACTCTGG
ATTTATAGCACATCCAACTTGGCTTCTGGAGTCCCTGCTCGCTTCAGTG
GCAGTGGGTCTGGGACCTCTTACTCTCTCACAATCAGCAGTGTGGAGGC
TGAAGATGCTGCCACTTATTACTGCCAGCAGTACAGTGGTTACCCACTC
ACGTTCGGAGGGGGGACCAAGCTGGAAATAAAA

As used herein, the term TNT-2 refers to an antibody comprising an amino acid sequence with CDRs that share at least 70%, or alternatively at least 80% amino acid sequence identity, preferably 90% sequence identity, more preferably at least 95% sequence identity with any one of the CDRs, preferably at least one of the CDR3 regions, most preferably both of the CDR3 regions, disclosed below.

TNT-2 CDHR1, SEQ ID NO: 9:
GYSFTGYY
TNT-2 CDHR2, SEQ ID NO: 10:
INPYNGAT
TNT-2 CDHR3, SEQ ID NO: 11:
ARLDRGDY
TNT-2 CDLR1, SEQ ID NO: 12:
ENVVTY
TNT-2 CDLR2, SEQ ID NO: 13:
GAS
TNT-2 CDLR3, SEQ ID NO: 14:
GQGYSYPYT
TNT-2 Heavy Chain Variable Region Sequence,
SEQ ID NO: 15:
GAGGTACAGCTGCAGCAGTCTGGACCTGAGCTGGTGAAGCCTGGGGCTT
CAGTGAAGATATCCTGCAAGGCTTCTGGTTACTCATTCACTGGCTACTA
CATGCACTGGGTGAAGCAAAGCCATGTAAAGAGCCTTGAGTGGATTGGA
CGTATTAATCCTTACAATGGTGCTACTAGCTACAACCAGAATTTCAAGG
ACAAGGCCAGCTTGACTGTAGATAAGTCCTCCAGCACAGCCTACATGGA
GCTCCACAGCCTGACATCTGAGGACTCTGCAGTCTATTACTGTGCAAGA
CTAGACCGGGGGGACTACTGGGGTCAAGGAACCTCAGTCACCGTCTCCT
CA
TNT-2 Light Chain Variable Region Sequence,
SEQ ID NO: 16:
AACATTGTAATGACCCAATCTCCCAAATCCATGTCCATGTCAGTAGGAG
AGAGGGTCACCTTGACCTGCAAGGCCAGTGAGAATGTGGTTACTTATGT
TTCCTGGTATCAACAGAAACCAGAGCAGTCTCCTAAACTGCTGATATAC
GGGGCATCCAACCGGTACACTGGGGTCCCCGATCGCTTCACAGGCAGTG
GATCTGCAACAGATTTCACTCTGACCATCAGCAGTGTGCAGGCTGAAGA
CCTTGCAGATTATCACTGTGGACAGGGTTACAGCTATCCGTACACGTTC
GGAGGGGGGACAAAGTTGGAAATAAAACGTACG

As used herein, the term TNT-3 refers to an antibody comprising an amino acid sequence with CDRs that share at least 70%, or alternatively at least 80% amino acid sequence identity, preferably 90% sequence identity, more preferably at least 95% sequence identity with any one of the CDRs, preferably at least one of the CDR3 regions, most preferably both of the CDR3 regions, disclosed below.

TNT-3 CDHR1, SEQ ID NO: 17:
GYTFTRYW
TNT-3 CDHR2, SEQ ID NO: 18:
IYPGNSDT
TNT-3 CDHR3, SEQ ID NO: 19:
ARGEEIGVRRWFAY
TNT-3 CDLR1, SEQ ID NO: 20:
QSISNY
TNT-3 CDLR2, SEQ ID NO: 21:
YAS
TNT-3 CDLR3, SEQ ID NO: 22:
QQSNSWPLT
TNT-3 Heavy Chain Variable Region Sequence,
SEQ ID NO: 23:
CAGGTCCAACTGCAGCAGTCAGGAGCTGAACTGGTCAAGACTGGGGCCT
CAGTGAAGATGTCCTGCAAGGCTTCTGGCTACACCTTTACCAGATACTG
GATGCACTGGGTAAAACAGAGGCCTGGACAGGCTCTGGAATGGATTGGC
GCTATTTATCCTGGAAATAGTGATACTAGCTACTACCAGAAGTTCAAGG
GCAAGGCCAAACTGACTGCAGTCACATCTGCCAGCACTGCCTACATGGA
GCTCAGCAGCCTGACATCTGAGGACTCTGCCGTCTATTACTGTGCAAGA
GGGGAGGAAATAGGGGTACGACGCTGGTTTGCTTACTGGGGCCAAGGGA
CTCTGGTCACTGTCTCTGCA
TNT-3 Light Chain Variable Region Sequence,
SEQ ID NO: 24:
GATATTGTGC TAACTCAGTC TCCAGCCACC CTGTCTGTGA
CTCCAGGAGA TAGAGTCAGT CTTTCCTGCA GGGCCAGGCA
AAGTATTAGC AACTACCTAC ACTGGTATCA ACAAAAATCA
CATGAGTCTC CAAGGCTTCT CATCAAGTAT GCTTCCCAGT
CCATCTCTGG CATCCCCTCC AGGTTCAGTG GCAGTGGATC
AGGGACAGAT TTCACTCTCA GTATCAACAG TGTGGAGACT
GAAGATTTTG GAATGTATTT CTGTCAACAG AGTAACAGCT
GGCCGCTCAC GTTCGGTGCT GGGACCAAGC TGGAAATAAA
A

As used herein, the term NHS76 refers to an antibody comprising an amino acid sequence with CDRs that share at least 70%, or alternatively at least 80% amino acid sequence identity, preferably 90% sequence identity, more preferably at least 95% sequence identity with any one of the CDRs, preferably at least one of the CDR3 regions, most preferably both of the CDR3 regions, disclosed below and in U.S. Pat. No. 6,827,925.

NHS76 CDHR1, SEQ ID NO: 25:
SGYYWG
NHS76 CDHR2, SEQ ID NO: 26:
SIYHSGSTYYNPSLKS
NHS76 CDHR3, SEQ ID NO: 27:
GKWSKFDY
NHS76 CDLR1, SEQ ID NO: 28:
QGDSLRSYYAS
NHS76 CDLR2, SEQ ID NO: 29:
GKNNRPS
NHS76 CDLR3, SEQ ID NO: 30:
NSRDSSGNHVV
NHS76 Heavy Chain Variable Region Sequence,
SEQ ID NO: 31:
CAGGTGCAGC TGCAGGAGTC CGGCCCAGGA CTGGTGAAGC
CTTCGGAGAC CCTGTCCCTC ACCTGCGCTG TCTCTGGTTA
CTCCATCAGC AGTGGTTACT ACTGGGGCTG GATTCGGCAG
CCCCCAGGGA AGGGGCTGGA GTGGATTGGG AGTATCTATC
ATAGTGGGAG CACCTACTAC AACCCGTCCC TCAAGAGTCG
AGTCACCATA TCAGTAGACA CGTCCAAGAA CCAGTTCTCC
CTGAAGCTGA GCTCTGTGAC CGCCGCAGAC ACGGCCGTGT
ATTACTGTGC AAGAGGGAAG TGGTCGAAGT TTGACTATTG
GGGCCAAGGC ACCCTGGTCA CCGTCTCTTC A
NHS76 Light Chain Variable Region Sequence,
SEQ ID NO: 32:
TCCTCTGAGC TGACTCAGGA CCCTGCTGTG TCTGTGGCCT
TGGGACAGAC AGTCAGGATC ACATGCCAAG GAGACAGCCT
CAGAAGCTAT TATGCAAGCT GGTACCAGCA GAAGCCAGGA
CAGGCCCCTG TACTTGTCAT CTATGGTAAA AACAACCGGC
CCTCAGGGAT TCCAGACCGA TTCTCTGGCT CCAGCTCAGG
AAACACAGCT TCCTTGACCA TCACTGGGGC TCAGGCGGAA
GATGAGGCTG ACTATTACTG TAACTCCCGG GACAGCAGTG
GTAACCATGT GGTATTCGGC GGAGGGACCA AGCTGACCGT
CCTA

As used herein, the term “autologous,” in reference to cells refers to cells that are isolated and infused back into the same subject (recipient or host). “Allogeneic” refers to non-autologous cells.

As used herein, the term “B cell,” refers to a type of lymphocyte in the humoral immunity of the adaptive immune system. B cells principally function to make antibodies, serve as antigen presenting cells, release cytokines, and develop memory B cells after activation by antigen interaction. B cells are distinguished from other lymphocytes, such as T cells, by the presence of a B-cell receptor on the cell surface. B cells may either be isolated or obtained from a commercially available source. Non-limiting examples of commercially available B cell lines include lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™), DS-1 (ATCC® CRL-11102™) EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), AND SUP-B15 (ATCC CRL-1929). Further examples include but are not limited to cell lines derived from anaplastic and large cell lymphomas, e.g., DEL, DL-40, FE-PD, JB6, Karpas 299, Ki-JK, Mac-2A Ply1, SR-786, SU-DHL-1, -2, -4, -5, -6, -7, -8, -9, -10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, SU/RH-HD-1. Non-limiting exemplary sources for such commercially available cell lines include the American Type Culture Collection, or ATCC, (http://www.atcc.org/) and the German Collection of Microorganisms and Cell Cultures (https://www.dsmz.de/).

The term “chimeric antigen receptor” (CAR), as used herein, refers to a fused protein comprising an extracellular domain capable of binding to an antigen, a transmembrane domain derived from a polypeptide different from a polypeptide from which the extracellular domain is derived, and at least one intracellular domain. The “chimeric antigen receptor (CAR)” is sometimes called a “chimeric receptor”, a “T-body”, or a “chimeric immune receptor (CIR).” The “extracellular domain capable of binding to an antigen” means any oligopeptide or polypeptide that can bind to a certain antigen. The “intracellular domain” or “intracellular signaling domain” means any oligopeptide or polypeptide known to function as a domain that transmits a signal to cause activation or inhibition of a biological process in a cell. In certain embodiments, the intracellular domain may comprise, alternatively consist essentially of, or yet further comprise one or more costimulatory signaling domains in addition to the primary signaling domain. The “transmembrane domain” means any oligopeptide or polypeptide known to span the cell membrane and that can function to link the extracellular and signaling domains. A chimeric antigen receptor may optionally comprise a “hinge domain” which serves as a linker between the extracellular and transmembrane domains. Non limiting examples of such domains are provided herein, e.g.:

Hinge domain: IgG1 heavy chain hinge sequence,
SEQ ID NO: 59:
CTCGAGCCCAAATCTTGTGACAAAACTCACACATGCCCACCGTGCCCG
Transmembrane domain: CD28 transmembrane region
SEQ ID NO: 60:
TTTTGGGTGCTGGTGGTGGTTGGTGGAGTCCTGGCTTGCTATAGCTTGC
TAGTAACAGTGGCCTTTATTATTTTCTGGGTG
Intracellular domain: 4-1BB co-stimulatory
signaling region, SEQ ID NO: 61:
AAACGGGGCAGAAAGAAACTCCTGTATATATTCAAACAACCATTTATGA
GACCAGTACAAACTACTCAAGAGGAAGATGGCTGTAGCTGCCGATTTCC
AGAAGAAGAAGAAGGAGGATGTGAACTG
Intracellular domain: CD28 co-stimulatory
signaling region, SEQ ID NO: 62:
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCC
Intracellular domain: CD3 zeta signaling region,
SEQ ID NO: 63:
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGCTAA

As used herein, the term “CD8 α hinge domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α hinge domain sequence as shown herein. The example sequences of CD8 α hinge domain for human, mouse, and other species are provided in Pinto, R. D. et al. (2006) Vet. Immunol. Immunopathol. 110:169-177. The sequences associated with the CD8 α hinge domain are provided in Pinto, R. D. et al. (2006) Vet. Immunol. Immunopathol. 110:169-177. Non-limiting examples of such include:

Human CD8 alpha hinge domain, SEQ. ID NO: 33:
PAKPTTTPAPRPPTPAPTIASQPLSLRPEACRPAAGGAVHTRGLDFACD
IY
Mouse CD8 alpha hinge domain, SEQ. ID NO: 34:
KVNSTTTKPVLRTPSPVHPTGTSQPQRPEDCRPRGSVKGTGLDFACDIY
Cat CD8 alpha hinge domain, SEQ. ID NO: 35:
PVKPTTTPAPRPPTQAPITTSQRVSLRPGTCQPSAGSTVEASGLDLSCD
IY

As used herein, the term “CD8 α transmembrane domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD8 α transmembrane domain sequence as shown herein. The fragment sequences associated with the amino acid positions 183 to 203 of the human T-cell surface glycoprotein CD8 alpha chain (GenBank Accession No: NP 001759.3), or the amino acid positions 197 to 217 of the mouse T-cell surface glycoprotein CD8 alpha chain (GenBank Accession No: NP 001074579.1), and the amino acid positions 190 to 210 of the rat T-cell surface glycoprotein CD8 alpha chain (GenBank Accession No: NP 113726.1) provide additional example sequences of the CD8 α transmembrane domain. The sequences associated with each of the listed accession numbers are provided as follows:

Human CD8 alpha transmembrane domain,
SEQ. ID NO: 36:
IYIWAPLAGTCGVLLLSLVIT
Mouse CD8 alpha transmembrane domain,
SEQ. ID NO: 37:
IWAPLAGICVALLLSLIITLI
Rat CD8 alpha transmembrane domain,
SEQ. ID NO: 38:
IWAPLAGICAVLLLSLVITLI

As used herein, the term “CD28 transmembrane domain” refers to a specific protein fragment associated with this name and any other molecules that have analogous biological function that share at least 70%, or alternatively at least 80% amino acid sequence identity, at least 90% sequence identity, or alternatively at least 95% sequence identity with the CD28 transmembrane domain sequence as shown herein. The fragment sequences associated with the GenBank Accession Nos: XM_006712862.2 and XM_009444056.1 provide additional, non-limiting, example sequences of the CD28 transmembrane domain. The sequences associated with each of the listed accession numbers are provided as follows the sequence encoded by SEQ ID NO: 60.

As used herein, the term “4-1BB costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the 4-1BB costimulatory signaling region sequence as shown herein. Non-limiting example sequences of the 4-1BB costimulatory signaling region are provided in U.S. Publication 20130266551A1 (filed as U.S. application Ser. No. 13/826,258), such as the exemplary sequence provided below:

4-1BB costimulatory signaling region,
SEQ ID NO: 39:
KRGRKKLLYIFKQPFMRPVQTTQEEDGCSCRFPEEEEGGCEL

As used herein, the term “CD28 costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the CD28 costimulatory signaling region sequence shown herein. The example sequences CD28 costimulatory signaling domain are provided in U.S. Pat. No. 5,686,281; Geiger, T. L. et al., Blood 98: 2364-2371 (2001); Hombach, A. et al., J Immunol 167: 6123-6131 (2001); Maher, J. et al. Nat Biotechnol 20: 70-75 (2002); Haynes, N. M. et al., J Immunol 169: 5780-5786 (2002); Haynes, N. M. et al., Blood 100: 3155-3163 (2002). Non-limiting examples include residues 114-220 of the below CD28 Sequence: MLRLLLALNL FPSIQVTGNK ILVKQSPMLV AYDNAVNLSC KYSYNLFSRE FRASLHKGLDSAVEVCVVYG NYSQQLQVYS KTGFNCDGKL GNESVTFYLQ NLYVNQTDIY FCKIEVMYPPPYLDNEKSNG TIIHVKGKHL CPSPLFPGPS KPFWVLVVVG GVLACYSLLVTVAFIIFWVR SKRSRLLHSD YMNMTPRRPG PTRKHYQPYA PPRDFAAYRS (SEQ ID NO: 40), and equivalents thereof.

As used herein, the term “ICOS costimulatory signaling region” refers to a specific protein fragment associated with this name and any other molecules that have analogous biological function that share at least 70%, or alternatively at least 80% amino acid sequence identity, preferably 90% sequence identity, more preferably at least 95% sequence identity with the ICOS costimulatory signaling region sequence as shown herein. Non-limiting example sequences of the ICOS costimulatory signaling region are provided in U.S. Publication 2015/0017141A1 the exemplary polynucleotide sequence provided below.

ICOS costimulatory signaling region,
SEQ ID NO: 64:
ACAAAAAAGA AGTATTCATC CAGTGTGCAC GACCCTAACG
GTGAATACAT GTTCATGAGA GCAGTGAACA CAGCCAAAAA
ATCCAGACTC ACAGATGTGA CCCTA

As used herein, the term “OX40 costimulatory signaling region” refers to a specific protein fragment associated with this name and any other molecules that have analogous biological function that share at least 70%, or alternatively at least 80% amino acid sequence identity, or alternatively 90% sequence identity, or alternatively at least 95% sequence identity with the OX40 costimulatory signaling region sequence as shown herein. Non-limiting example sequences of the OX40 costimulatory signaling region are disclosed in U.S. Publication 2012/20148552A1, and include the exemplary sequence provided below.

OX40 costimulatory signaling region,
SEQ ID NO: 65:
AGGGACCAG AGGCTGCCCC CCGATGCCCA CAAGCCCCCT
GGGGGAGGCA GTTTCCGGAC CCCCATCCAA GAGGAGCAGG
CCGACGCCCA CTCCACCCTG GCCAAGATC

As used herein, the term “CD3 zeta signaling domain” refers to a specific protein fragment associated with this name and any other molecules that have analogous biological function that share at least 70%, or alternatively at least 80% amino acid sequence identity, preferably 90% sequence identity, more preferably at least 95% sequence identity with the CD3 zeta signaling domain sequence as shown herein. Non-limiting example sequences of the CD3 zeta signaling domain are provided in U.S. application Ser. No. 13/826,258, e.g.:

(SEQ ID NO: 41)

RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

A “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. For example, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions disclosed herein. Aspects defined by each of these transition terms are within the scope of the present disclosure.

The term “consensus sequence” as used herein refers to an amino acid or nucleic acid sequence that is determined by aligning a series of multiple sequences and that defines an idealized sequence that represents the predominant choice of amino acid or base at each corresponding position of the multiple sequences. Depending on the sequences of the series of multiple sequences, the consensus sequence for the series can differ from each of the sequences by zero, one, a few, or more substitutions. Also, depending on the sequences of the series of multiple sequences, more than one consensus sequence may be determined for the series. The generation of consensus sequences has been subjected to intensive mathematical analysis. Various software programs can be used to determine a consensus sequence.

“Cytoreductive therapy,” as used herein, includes but is not limited to chemotherapy, cryotherapy, and radiation therapy. Agents that act to reduce cellular proliferation are known in the art and widely used. Chemotherapy drugs that kill cancer cells only when they are dividing are termed cell-cycle specific. These drugs include agents that act in S-phase, including topoisomerase inhibitors and anti-metabolites.

Toposiomerase inhibitors are drugs that interfere with the action of topoisomerase enzymes (topoisomerase I and II). During the process of chemo treatments, topoisomerase enzymes control the manipulation of the structure of DNA necessary for replication, and are thus cell cycle specific. Examples of topoisomerase I inhibitors include the camptothecan analogs listed above, irinotecan and topotecan. Examples of topoisomerase II inhibitors include amsacrine, etoposide, etoposide phosphate, and teniposide.

Antimetabolites are usually analogs of normal metabolic substrates, often interfering with processes involved in chromosomal replication. They attack cells at very specific phases in the cycle. Antimetabolites include folic acid antagonists, e.g., methotrexate; pyrimidine antagonist, e.g., 5-fluorouracil, foxuridine, cytarabine, capecitabine, and gemcitabine; purine antagonist, e.g., 6-mercaptopurine and 6-thioguanine; adenosine deaminase inhibitor, e.g., cladribine, fludarabine, nelarabine and pentostatin; and the like.

Plant alkaloids are derived from certain types of plants. The vinca alkaloids are made from the periwinkle plant (Catharanthus rosea). The taxanes are made from the bark of the Pacific Yew tree (taxus). The vinca alkaloids and taxanes are also known as antimicrotubule agents. The podophyllotoxins are derived from the May apple plant. Camptothecan analogs are derived from the Asian “Happy Tree” (Camptotheca acuminata). Podophyllotoxins and camptothecan analogs are also classified as topoisomerase inhibitors. The plant alkaloids are generally cell-cycle specific.

Examples of these agents include vinca alkaloids, e.g., vincristine, vinblastine and vinorelbine; taxanes, e.g., paclitaxel and docetaxel; podophyllotoxins, e.g., etoposide and tenisopide; and camptothecan analogs, e.g., irinotecan and topotecan.

Cryotherapy includes, but is not limited to, therapies involving decreasing the temperature, for example, hypothermic therapy.

Radiation therapy includes, but is not limited to, exposure to radiation, e.g., ionizing radiation, UV radiation, as known in the art. Exemplary dosages include, but are not limited to, a dose of ionizing radiation at a range from at least about 2 Gy to not more than about 10 Gy and/or a dose of ultraviolet radiation at a range from at least about 5 J/m² to not more than about 50 J/m², usually about 10 J/m².

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I.

An “effective amount” or “efficacious amount” refers to the amount of an agent, or combined amounts of two or more agents, that, when administered for the treatment of a mammal or other subject, is sufficient to effect such treatment for the disease. The “effective amount” will vary depending on the agent(s), the disease and its severity and the age, weight, etc., of the subject to be treated.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, the term “enhancer”, as used herein, denotes sequence elements that augment, improve or ameliorate transcription of a nucleic acid sequence irrespective of its location and orientation in relation to the nucleic acid sequence to be expressed.

An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, polypeptide or nucleic acid, intends those having minimal homology while still maintaining desired structure or functionality. Unless specifically recited herein, it is contemplated that any polynucleotide, polypeptide or protein mentioned herein also includes equivalents thereof. For example, an equivalent intends at least about 70% homology or identity, or at least 80% homology or identity and alternatively, or at least about 85%, or alternatively at least about 90%, or alternatively at least about 95%, or alternatively 98% percent homology or identity and exhibits substantially equivalent biological activity to the reference protein, polypeptide or nucleic acid. Alternatively, when referring to polynucleotides, an equivalent thereof is a polynucleotide that hybridizes under stringent conditions to the reference polynucleotide or its complement.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

As used herein, “homology” or “identical”, percent “identity” or “similarity”, when used in the context of two or more nucleic acids or polypeptide sequences, refers to two or more sequences or subsequences that are the same or have a specified percentage of nucleotides or amino acid residues that are the same, e.g., at least 60% identity, preferably at least 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein). Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. The terms “homology” or “identical”, percent “identity” or “similarity” also refer to, or can be applied to, the complement of a test sequence. The terms also include sequences that have deletions and/or additions, as well as those that have substitutions. As described herein, the preferred algorithms can account for gaps and the like. Preferably, identity exists over a region that is at least about 25 amino acids or nucleotides in length, or more preferably over a region that is at least 50-100 amino acids or nucleotides in length. An “unrelated” or “non-homologous” sequence shares less than 40% identity, or alternatively less than 25% identity, with one of the sequences disclosed herein.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, the term “immunoregulatory molecule” may refer to any molecule that may regulate or directly influence immune responses, including but not limited to chemokines such as CCL2, CCL5, CCL14, CCL19, CCL20, CXCL8, CXCL13, and LEC; lymphokines and cytokines such as interleukins (e.g., IL-2, IL-7, IL-12, IL-15, IL-18, IL-21, etc.), interferons α, β and γ, factors stimulating cell growth (e.g., GM-CSF), and other factors (e.g., tumor necrosis factors, DC-SIGN, M1P1α, M1P1β, TGF-β or TNF); factors that provide co-stimulatory signals for T-cell activation such as B7 molecules and CD40; accessory molecules such as CD83; proteins involved in antigen processing and presentation such as TAP1/TAP2 transporter proteins, proteosome molecules such as LMP2 and LMP7, heat shock proteins such as gp96, HSP70 and HSP90, and MHC or HLA molecules; and biological equivalents thereof. Non-limiting examples of immunoregulatory molecules are disclosed herein.

As used herein, the term “B7.1” (also known as B7; BB1; B7-1; CD80; LAB7; CD28LG; CD28LG1) refers to a specific molecule associated with this name and any other molecules that have analogous biological function that share at least 80% amino acid sequence identity, preferably 90% sequence identity, more preferably at least 95% sequence identity with B7.1. Examples of the B7.1 sequence are provided herein. In addition, the sequences associated with GenBank Accession Nos. NM_005191.3 and NP_005182.1 are exemplary.

### Non-Limiting Examples Include NP_005182.1, SEQ ID NO: 42:

MGHTRRQGTS PSKCPYLNFF QLLVLAGLSH FCSGVIHVTK
EVKEVATLSC GHNVSVEELA QTRIYWQKEK KMVLTMMSGD
MNIWPEYKNR TIFDITNNLS IVILALRPSD EGTYECVVLK
YEKDAFKREH LAEVTLSVKA DFPTPSISDF EIPTSNIRRI
ICSTSGGFPE PHLSWLENGE ELNAINTTVS QDPETELYAV
SSKLDFNMTT NHSFMCLIKY GHLRVNQTFN WNTTKQEHFP
DNLLPSWAIT LISVNGIFVI CCLTYCFAPR CRERRRNERL
RRESVRPV

In some embodiments where B7.1 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (http://www.linscottsdirectory.com/).

As used herein, the term “CCL19” (also known as ELC; CKb11; MIP3B; MIP-3b; SCYA19) refers to a specific molecule associated with this name and any other molecules that have analogous biological function that share at least 80% amino acid sequence identity, preferably 90% sequence identity, more preferably at least 95% sequence identity with CCL19. Examples of the CCL19 sequence are provided herein. In addition, the sequences associated with GenBank Accession Nos. NC_000009.11 NC_018920.2 NT_008413.19, NP_006265.1 are exemplary.

### Non-Limiting Examples Include NP_006265.1, SEQ ID NO: 43:

MALLLALSLL VLWTSPAPTL SGTNDAEDCC LSVTQKPIPG
YIVRNFHYLL IKDGCRVPAV VFTTLRGRQL CAPPDQPWVE
RIIQRLQRTS AKMKRRSS

In some embodiments where CCL19 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (http://www.linscottsdirectory.com/).

As used herein, the term “CCL20” (also known as CKb4; LARC; ST38; MIP3A; Exodus; MIP-3a; SCYA20; MIP-3-alpha) refers to a specific molecule associated with this name and any other molecules that have analogous biological function that share at least 80% amino acid sequence identity, preferably 90% sequence identity, more preferably at least 95% sequence identity with CCL20. Examples of the CCL20 sequence are provided herein. In addition, the sequences associated with GenBank Accession Nos. NC_000002.11 NC_018913.2 NT_005403.18, NP_001123518.1, and NP_004582.1 are exemplary.

### An Examples Include NP_004582.1, SEQ ID NO: 44:

MCCTKSLLLA ALMSVLLLHL CGESEAASNF DCCLGYTDRI
LHPKFIVGFT RQLANEGCDI NAIIFHTKKK LSVCANPKQT
WVKYIVRLLS KKVKNM
and
NP_001123518.1, SEQ ID NO: 45:
MCCTKSLLLA ALMSVLLLHL CGESEASNFD CCLGYTDRIL
HPKFIVGFTR QLANEGCDIN AIIFHTKKKL SVCANPKQTW
VKYIVRLLSK KVKNM

In some embodiments where CCL20 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see web address: linscottsdirectory.com, last accessed on Apr. 9, 2015).

As used herein, the term “CD40L” (also known as IGM; IMID3; TRAP; gp39; CD154; CD40LG; HIGM1; T-BAM; TNFSF5; hCD40L) refers to a specific molecule associated with this name and any other molecules that have analogous biological function that share at least 80% amino acid sequence identity, preferably 90% sequence identity, more preferably at least 95% sequence identity with CD40L. Examples of the CD40L sequence are provided herein. In addition, the sequences associated with GenBank Accession Nos. NC_000023.10, NC_018934.2, NT_011786.17, NP_000065.1 are exemplary.

### Non-Limiting Examples Include NP_000065.1, SEQ ID NO: 46:

MIETYNQTSP RSAATGLPIS MKIFMYLLTV FLITQMIGSA
LFAVYLHRRL DKIEDERNLH EDFVFMKTIQ RCNTGERSLS
LLNCEEIKSQ FEGFVKDIML NKEETKKENS FEMQKGDQNP
QIAAHVISEA SSKTTSVLQW AEKGYYTMSN NLVTLENGKQ
LTVKRQGLYY IYAQVTFCSN REASSQAPFI ASLCLKSPGR
FERILLRAAN THSSAKPCGQ QSIHLGGVFE LQPGASVFVN
VTDPSQVSHG TGFTSFGLLK L

In some embodiments where CD40L is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see web address at linscottsdirectory.com, last accessed on Apr. 9, 2015).

As used herein, the term “CD137L” (also known as TNFSF9; 4-1BB-L) refers to a specific molecule associated with this name and any other molecules that have analogous biological function that share at least 80% amino acid sequence identity, preferably 90% sequence identity, more preferably at least 95% sequence identity with CD137L. Examples of the CD137L sequence are provided herein. In addition, the protein associated with GenBank Accession Nos. NC_000019.9, NT_011295.12, NC_018930.2, and NP_003802.1 are exemplary.

Non-Limiting Examples Include NP_003802.1, SEQ ID NO: 47:

MEYASDASLD PEAPWPPAPR ARACRVLPWA LVAGLLLLLL
LAAACAVFLA CPWAVSGARA SPGSAASPRL REGPELSPDD
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE

In some embodiments where CD137L is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see web address at: linscottsdirectory.com, last accessed on Apr. 9, 2015).

As used herein, the term “GITRL” (also known as TNFSF18; TL6; AITRL; hGITRL) refers to a specific molecule associated with this name and any other molecules that have analogous biological function that share at least 80% amino acid sequence identity, preferably 90% sequence identity, more preferably at least 95% sequence identity with GITRL. Examples of the GITRL sequence are provided herein. In addition, the protein associated with GenBank Accession Nos. NC_000001.10, NC_018912.2, NT_004487.20, and NP_005083.2 are exemplary.

Non-Limiting Examples Include NP_005083.2, SEQ ID NO: 48:

MTLHPSPITC EFLFSTALIS PKMCLSHLEN MPLSHSRTQG
AQRSSWKLWL FCSIVMLLFL CSFSWLIFIF LQLETAKEPC
MAKFGPLPSK WQMASSEPPC VNKVSDWKLE ILQNGLYLIY
GQVAPNANYN DVAPFEVRLY KNKDMIQTLT NKSKIQNVGG
TYELHVGDTI DLIFNSEHQV LKNNTYWGII LLANPQFIS

In some embodiments where GITRL is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see web address at linscottsdirectory.com, last accessed on Apr. 9, 2015).

As used herein, the term “GM-CSF” (also known as granulocyte-macrophage colony stimulating factor; CSF2) refers to a specific molecule associated with this name and any other molecules that have analogous biological function that share at least 80% amino acid sequence identity, preferably 90% sequence identity, more preferably at least 95% sequence identity with GM-CSF. Examples of the GM-CSF sequence are provided herein. In addition, the protein sequence associated with UniProt Reference No. P04141—CSF2_HUMAN:

(SEQ ID NO: 49)

MWLQSLLLLG TVACSISAPA RSPSPSTQPW EHVNAIQEAR
RLLNLSRDTA AEMNETVEVI SEMFDLQEPT CLQTRLELYK
QGLRGSLTKL KGPLTMMASH YKQHCPPTPE TSCATQIITF
ESFKENLKDF LLVIPFDCWE PVQE

In some embodiments where GM-CSF is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (http://www.linscottsdirectory.com).

As used herein, the term “IL-12” (also known as “interleukin 12”) refers to a specific molecule associated with this name and any other molecules that have analogous biological function that share at least 80% amino acid sequence identity, preferably 90% sequence identity, more preferably at least 95% sequence identity with IL-12. Examples of the IL-12 sequence are provided herein, and include but are not limited to mature form IL-12 and variants and fragments thereof, such as single chain IL-12, IL-12A (GenBank Accession Nos. NC_000003.11 NT_005612.17 NC_018914.2), and IL-12B (GenBank Accession Nos. NC_000005.9 NC_018916.2 NT_023133.14). The protein sequences associated with the sequences disclosed in U.S. Pat. No. 8,556,882 are exemplary, for instance, the single chain IL-12 encoded by the sequence SEQ ID NO: 50. In some embodiments where IL-12 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (http://www.linscottsdirectory.com).

As used herein, the term “IL-2” (also known as “interleukin 2”) refers to a specific molecule associated with this name and any other molecules that have analogous biological function that share at least 80% amino acid sequence identity, preferably 90% sequence identity, more preferably at least 95% sequence identity with IL-2. A non-limiting example is SEQ ID NO: 51, which provides the full length sequence of native human IL-2:

APTSSSTKKT QLQLEHLLLD LQMILNGINN YKNPKLTRML
TFKFYMPKKA TELKHLQCLE EELKPLEEVL NLAQSKNFHL
RPRDLISNIN VIVLELKGSE TTFMCEYADE TATIVEFLNR
WITFCQSIIS TLT

The term “low-toxicity IL-2” refers to a modified version of IL-2 exhibiting analogous biological function but lower toxicity when administered to a subject. In some embodiments, low-toxicity IL-2 comprises a mutation with reduced vasopermeability compared to wild type IL-2. U.S. Pat. No. 7,371,371 discloses exemplary mutations in the permeability enhancing region of wild type IL-2 between amino acid positions 22 to 58 of human IL-2. Non-limiting examples include a mutation of R to W at position 38 in the human sequence. U.S. Pat. No. 7,371,371 further discloses low-toxicity IL-2 comprising a mutation at one or more positions outside the permeability enhancing region of IL-2.

As used herein, the term “IL-15” (also known as “interleukin 15”) refers to a specific molecule associated with this name and any other molecules that have analogous biological function that share at least 80% amino acid sequence identity, preferably 90% sequence identity, more preferably at least 95% sequence identity with IL-15. Examples of the IL-15 sequence are provided herein. In addition, the protein sequences associated with GenBank Accession Nos. NC_000004.11, NC_018915.2, NT_016354.20, NP_000576.1, NP_751915.1 are exemplary. Non-limiting examples the mature protein encoded by, SEQ ID NO: 52. In some embodiments where Il-15 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see linscottsdirectory.com, noted above).

As used herein, the term “IL-18” (also known as “interleukin 18,” IGIF, “interleukin 1 gamma,” IL1F4, IFN-Gamma-Inducing Factor, IL-1g) refers to a specific molecule associated with this name and any other molecules that have analogous biological function that share at least 80% amino acid sequence identity, preferably 90% sequence identity, more preferably at least 95% sequence identity with IL-18. Examples of the IL-18 sequence are provided herein. In addition, the protein sequences associated with GenBank Accession Nos. NC_000011.9, NC_018922.2, NT_033899.9, NP_001230140.1, NP_001553.1 are exemplary. Non-limiting examples the mature protein encoded by, SEQ ID NO: 53. In some embodiments where IL-18 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see linscottsdirectory.com, noted above).

As used herein, the term “IL-21” (also known as “interleukin 21”; Za11; CVID11) refers to a specific molecule associated with this name and any other molecules that have analogous biological function that share at least 80% amino acid sequence identity, preferably 90% sequence identity, more preferably at least 95% sequence identity with IL-21. Examples of the IL-21 sequence are provided herein. In addition, the protein sequences associated with GenBank Accession Nos. NC_000004.11, NC_018915.2, NT_016354.20, are exemplary. Non-limiting examples include the mature protein encoded by, SEQ ID NO: 54. In some embodiments where Il-21 is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see linscottsdirectory.com, noted above).

As used herein, the term “LEC” (also known as CCL16; LMC; NCC4; CKb12; HCC-4; LCC-1; Mtn-1; NCC-4; SCYL4; ILINCK; SCYA16) refers to a specific molecule associated with this name and any other molecules that have analogous biological function that share at least 80% amino acid sequence identity, preferably 90% sequence identity, more preferably at least 95% sequence identity with LEC. Examples of the LEC sequence are provided herein. In addition, the protein sequences associated with GenBank Accession Nos. NC_000017.10, NT_010783.16, NT_187614.1, NC_018928.2, NP_004581.1 are exemplary.

Non-limiting examples include NP_004581.1, SEQ ID NO: 55:

MKVSEAALSL LVLILIITSA SRSQPKVPEW VNTPSTCCLK
YYEKVLPRRL VVGYRKALNC HLPAIIFVTK RNREVCTNPN
DDWVQEYIKD PNLPLLPTRN LSTVKIITAK NGQPQLLNSQ

In some embodiments where LEC is administered as part of a composition, it may be either synthesized or purchased from any available commercial source. Such surces include, Santa Cruz Biosciences, Origene, and other sellers of purified proteins and modified versions thereof. A listing of commercial sources may be found on Linscott's Directory of Immunological & Biological Reagents (see linscottsdirectory.com, noted above).

As used herein, the term “OX40L” (also known as TNFSF4; GP34; CD252; TXGP1; CD134L; OX-40L) refers to a specific molecule associated with this name and any other molecules that have analogous biological function that share at least 80% amino acid sequence identity, preferably 90% sequence identity, more preferably at least 95% sequence identity with OX40L. Examples of the OX40L sequence are provided herein. In addition, the protein sequences associated with GenBank Accession Nos. NC_000001.10, NT_004487.20, NC_018912.2, NP_003317.1 are exemplary.

Non-limiting examples include NP_003317.1, SEQ ID NO: 56:

MERVQPLEEN VGNAARPRFE RNKLLLVASV IQGLGLLLCF
TYICLHFSAL QVSHRYPRIQ SIKVQFTEYK KEKGFILTSQ
KEDEIMKVQN NSVIINCDGF YLISLKGYFS QEVNISLHYQ
KDEEPLFQLK KVRSVNSLMV ASLTYKDKVY LNVTTDNTSL
DDFHVNGGEL ILIHQNPGEF CVL

In some embodiments where OX40L is administered as part of a composition, it may be either synthesized or purchased from any available commercial source. Such sources include, Santa Cruz Biosciences, Origene, and other sellers of purified proteins and modified versions thereof. A listing of commercial sources may be found on Linscott's Directory of Immunological & Biological Reagents (see linscottsdirectory.com, noted above).

The term “isolated” as used herein refers to molecules or biologicals or cellular materials being substantially free from other materials. In one aspect, the term “isolated” refers to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

As used herein, the term “isolated cell” generally refers to a cell that is substantially separated from other cells of a tissue.

“Immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells).

As used herein the term “linker sequence” relates to any amino acid sequence comprising from 1 to 10, or alternatively, 8 amino acids, or alternatively 6 amino acids, or alternatively 5 amino acids that may be repeated from 1 to 10, or alternatively to about 8, or alternatively to about 6, or alternatively about 5, or 4 or alternatively 3, or alternatively 2 times. For example, the linker may comprise up to 15 amino acid residues consisting of a pentapeptide repeated three times. In one aspect, the linker sequence is a (Glycine4Serine)3 flexible polypeptide linker (SEQ ID NO: 67) comprising three copies of gly-gly-gly-gly-ser (SEQ ID NO: 68).

A “normal cell corresponding to the tumor tissue type” refers to a normal cell from a same tissue type as the tumor tissue. A non-limiting example is a normal lung cell from a patient having lung tumor, or a normal colon cell from a patient having colon tumor.

As used herein, the term “NFAT regulatory polynucleotide” refers to a polynucleotide that interacts with the Nuclear Factor of Activated T-cells (“NFAT”) family of transcription factors. NFAT refers to a collection of calcineurin-dependent transcriptional factors expressed in most immune cells and plays a vital role in activation of gene transcription in T cells (Fric, J. et al. (2012) Blood 120(7):1380-1389). In particular, NFAT controls the transcription of a large number of genes during an immune response, most notably IL-2, IL-4, TNF-α, and IFN-γ (Fric, J. et al. (2012) Blood 120(7):1380-1389; Rao, A. et al. (1997) Annu Rev Immunol. 15:707-747; Hogan, P. G. et al. (2003) Genes Dev. 17(18):2205-2232). An example of a polynucleotide that interacts with NFAT is: GGAGGAAAAACTGTTTCATACAGAAGGCG (SEQ ID NO: 57) and equivalents thereof.

As used herein, the term “T cell,” refers to a type of lymphocyte that matures in the thymus. T cells play an important role in cell-mediated immunity and are distinguished from other lymphocytes, such as B cells, by the presence of a T-cell receptor on the cell surface. T-cells may either be isolated or obtained from a commercially available source. “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Non-limiting examples of commercially available T-cell lines include lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™), TALL-104 cytotoxic human T cell line (ATCC # CRL-11386). Further examples include but are not limited to mature T-cell lines, e.g., such as Deglis, EBT-8, HPB-MLp-W, HUT 78, HUT 102, Karpas 384, Ki 225, My-La, Se-Ax, SKW-3, SMZ-1 and T34; and immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); and cutaneous T-cell lymphoma lines, e.g., HuT78 (ATCC CRM-TIB-161), MJ[G11] (ATCC CRL-8294), HuT102 (ATCC TIB-162). Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein, the term “NK cell,” also known as natural killer cell, refers to a type of lymphocyte that originates in the bone marrow and play a critical role in the innate immune system. NK cells provide rapid immune responses against viral-infected cells, tumor cells or other stressed cell, even in the absence of antibodies and major histocompatibility complex on the cell surfaces. NK cells may either be isolated or obtained from a commercially available source. Non-limiting examples of commercial NK cell lines include lines NK-92 (ATCC® CRL-2407™), NK-92MI (ATCC® CRL-2408™). Further examples include but are not limited to NK lines HANK1, KHYG-1, NKL, NK-YS, NOI-90, and YT. Non-limiting exemplary sources for such commercially available cell lines include the American Type Culture Collection, or ATCC, (http://www.atcc.org/) and the German Collection of Microorganisms and Cell Cultures (https://www.dsmz.de/).

As used herein in reference to a regulatory polynucleotide, the term “operatively linked” refers to an association between the regulatory polynucleotide and the polynucleotide sequence to which it is linked such that, when a specific protein binds to the regulatory polynucleotide, the linked polynucleotide is transcribed.

As used herein, the term “overexpress” with respect to a cell, a tissue, or an organ expresses a protein to an amount that is greater than the amount that is produced in a control cell, a control issue, or an organ. A protein that is overexpressed may be endogenous to the host cell or exogenous to the host cell.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “purified” does not require absolute purity; rather, it is intended as a relative term. Thus, for example, a purified nucleic acid, peptide, protein, biological complexes or other active compound is one that is isolated in whole or in part from proteins or other contaminants. Generally, substantially purified peptides, proteins, biological complexes, or other active compounds for use within the disclosure comprise more than 80% of all macromolecular species present in a preparation prior to admixture or formulation of the peptide, protein, biological complex or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, biological complex or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

As used herein, the term “purification marker” refers to at least one marker useful for purification or identification. A non-exhaustive list of this marker includes His, lacZ, GST, maltose-binding protein, NusA, BCCP, c-myc, CaM, FLAG, GFP, YFP, cherry, thioredoxin, poly(NANP), V5, Snap, HA, chitin-binding protein, Softag 1, Softag 3, Strep, or S-protein. Suitable direct or indirect fluorescence marker comprise FLAG, GFP, YFP, RFP, dTomato, cherry, Cy3, Cy 5, Cy 5.5, Cy 7, DNP, AMCA, Biotin, Digoxigenin, Tamra, Texas Red, rhodamine, Alexa fluors, FITC, TRITC or any other fluorescent dye or hapten.

As used herein, the term “recombinant protein” refers to a polypeptide which is produced by recombinant DNA techniques, wherein generally, DNA encoding the polypeptide is inserted into a suitable expression vector which is in turn used to transform a host cell to produce the heterologous protein.

As used herein, the term “specific binding” means the contact between an antibody and an antigen with a binding affinity of at least 10⁻⁶ M. In certain aspects, antibodies bind with affinities of at least about 10⁻⁷ M, and preferably 10⁻⁸ M, 10⁻⁹ M, 10⁻¹⁰ m, 10⁻¹¹ M, or 10⁻¹² M.

A “solid tumor” is an abnormal mass of tissue that usually does not contain cysts or liquid areas. Solid tumors can be benign or malignant, metastatic or non-metastatic. Different types of solid tumors are named for the type of cells that form them. Examples of solid tumors include sarcomas, carcinomas, and lymphomas.

The term “transduce” or “transduction” as it is applied to the production of chimeric antigen receptor cells refers to the process whereby a foreign nucleotide sequence is introduced into a cell. In some embodiments, this transduction is done via a vector.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector.

As used herein, the term “WPRE” or “Woodchuck Hepatitis Virus (WHP) Post-transcriptional Regulatory Element” refers to a specific nucleotide fragment associated with this name and any other molecules that have analogous biological function that share at least 70%, or alternatively at least 80% amino acid sequence identity, preferably 90% sequence identity, more preferably at least 95% sequence identity with the WPRE sequence as shown herein. For example, WPRE refers to a region similar to the human hepatitis B virus posttranscriptional regulatory element (HBVPRE) present in the Woodchuck hepatitis virus genomic sequence (GenBank Accession No. J04514), and that the 592 nucleotides from position 1093 to 1684 of this genomic sequence correspond to the post-transcriptional regulatory region (Journal of Virology, Vol. 72, p. 5085-5092, 1998). The analysis using retroviral vectors revealed that WPRE inserted into the 3′-terminal untranslated region of a gene of interest increases the amount of protein produced by 5 to 8 folds. It has also been reported that the introduction of WPRE suppresses mRNA degradation (Journal of Virology, Vol. 73, p. 2886-2892, 1999). In a broad sense, elements such as WPRE that increase the efficiency of amino acid translation by stabilizing mRNAs are also thought to be enhancers.

The sequences associated with each of the above listed GenBank Accession Nos. and references are herein incorporated by reference.

### List of Abbreviations

CAR: chimeric antigen receptor
HLA: histocompatibility lymphocyte antigen
IL: interleukin
Ip: intraperitoneal
IRES: internal ribosomal entry site
LEC: Liver-expressed chemokine
MFI: mean fluorescence intensity
MOI: multiplicity of infection
PBMC: peripheral blood mononuclear cells
PBS: phosphate buffered saline
scFv: single chain variable fragment
TNT: tumor necrosis therapy
WPRE: woodchuck hepatitis virus post-transcriptional regulatory element

### MODES FOR CARRYING OUT THE DISCLOSURE

Administration of immunoregulatory molecules has been pursued as a cancer therapeutic. However, due to severe side effects associated with systemic administration (Giovarelli, M. et al. (2000) J Immunol. 164:3200-3206; Lasek, W. et al. (2014) Cancer Immunol Immunother. 63:419-435), obtaining high concentrations of these immunoregulatory molecules in relevant tumors in order to achieve an effective immune response has been difficult.

Due to the unprecedented results being recently obtained in B-cell lymphomas and leukemia's using autologous treatment with genetically engineered chimeric antigen receptor (CAR) T-cells (Maude, S. L. et al. (2014) New Engl. J. Med. 371:1507-1517; Porter, D. L. et al. (2011) New Engl. J. Med. 365:725-733), a number of laboratories have begun to apply this approach to solid tumors including ovarian cancer, prostate cancer, and pancreatic tumors. CAR modified T-cells combine the HLA-independent targeting specificity of a monoclonal antibody with the cytolytic activity, proliferation, and homing properties of activated T-cells, but do not respond to checkpoint suppression. Because of their ability to kill antigen expressing targets directly, CAR T-cells are highly toxic to any antigen positive cells or tissues making it a requirement to construct CARs with highly tumor specific antibodies. To date, CAR modified T-cells to human solid tumors have been constructed against the α-folate receptor, mesothelin, and MUC-CD, PSMA, and other targets but most have some off-target expression of antigen in normal tissues. These constructs have not shown the same exceptional results in patients emphasizing the need for additional studies to identify new targets and methods of CAR T-cell construction that can be used against solid tumors.

Thus, this disclosure provides a chimeric antigen receptor (CAR) comprising a binding domain specific to a TNT relevant antigen, that in some aspects, is the antigen binding domain of a TNT antibody and methods and compositions relating to the use and production thereof.

### Chimeric Antigen Receptors and Uses Thereof

I. Components

The present disclosure provides chimeric antigen receptors (CAR) that bind to a TNT relevant antigen, the CAR comprising, or consisting essentially of, or consisting of, a cell activation moiety comprising an extracellular, transmembrane, and intracellular domain. The extracellular domain comprises a target-specific binding element otherwise referred to as the antigen binding domain. The intracellular domain or cytoplasmic domain comprises a costimulatory signaling region and a zeta chain portion. The CAR may optionally further comprise a spacer domain of up to 300 amino acids, preferably 10 to 100 amino acids, more preferably 25 to 50 amino acids.

Antigen Binding Domain.

In certain aspects, the present disclosure provides a CAR that comprises, or alternatively consists essentially thereof, or yet further consists of an antigen binding domain specific to a TNT-relevant antigen. TNT is an acronym for tumor necrosis therapy. A “tumor necrosis therapy (TNT) relevant antigen” is an antigen where the whole antigen, epitope, or fragment thereof is retained by a dying cell and where the whole antigen, epitope, or fragment thereof would not normally be exposed but for cell death. Non-limiting examples of such TNT relevant antigens are DNA or DNA/histone targets usually made accessible only after cell death; for example, TNT-1 binds to a portion of histone H1. Other TNT-relevant antigens that are also histone targets include but are not limited to H2A, H2B, H3, H4, H5, and/or other human or animal histones. Other TNT relevant antigens include, but are not limited to, single stranded DNA and heterochromatic DNA. The antigen binding domains can comprise, consist essentially of, or yet further consist of the antigen binding domains of the antibodies TNT-1, TNT-2, TNT-3, or NHS76.

In some embodiments, the antigen binding domain comprises, or alternatively consists essentially thereof, or yet consists of the antigen binding domain of a TNT antibody or an antibody that binds a TNT-relevant antigen. Monoclonal antibodies that specifically bind these antigens are commercially available, see for example the web address biocompare.com/pfu/110447/soids/123510/Antibodies/TNT, last accessed on Apr. 10, 2015). In one aspect, the antigen binding domain comprises the heavy chain variable region and the light chain variable region of a TNT antibody. In non-limiting embodiments, the heavy chain variable region and light chain variable region of a TNT antibody comprises, or alternatively consists essentially thereof, or yet consists of the antigen binding domain the TNT antibody.

In some embodiments, the heavy chain variable region comprises a CDRH1 sequence comprising, or alternatively consisting essentially of, or yet further consisting of, an amino acid sequence beginning with any one of the following sequences: (i) GFSLTDYG (SEQ ID NO: 1), (ii) GYSFTGYY (SEQ ID NO: 9), (iii) GYTFTRYW (SEQ ID NO: 17), (iv) SGYYWG (SEQ ID NO: 25), or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises a CDRH2 sequence comprising, or alternatively consisting essentially of, or yet further consisting of, an amino acid sequence beginning with any one of the following sequences: (i) IWGGGST (SEQ ID NO: 2), (ii) INPYNGAT (SEQ ID NO: 10), (iii) IYPGNSDT (SEQ ID NO: 18), (iv) SIYHSGSTYYNPSLKS (SEQ ID NO: 26), or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises a CDRH3 sequence comprising, or alternatively consisting essentially of, or yet further consisting of, an amino acid sequence beginning with any one of the following sequences: (i) AKEKRRGYYYAMDY (SEQ ID NO: 3), (ii) ARLDRGDY (SEQ ID NO: 11), (iii) ARGEEIGVRRWFAY (SEQ ID NO: 19), (iv) GKWSKFDY (SEQ ID NO: 27), or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the heavy chain variable region comprises, or alternatively consists essentially of, or yet further consists of, the polypeptide encoded by the below noted polynucleotide sequences: CAGGTGCAGCTGAAGGAGTCAGGACCTGGCCTGGTGGCGCCCTCACAGAGCCTG TCCATCACATGCACTGTCTCAGGGTTCTCATTAACCGACTATGGTGTAAGGTGGA TTCGCCAGCCTCCAGGAAAGGGTCTGGAGTGGCTGGGAGTAATATGGGGTGGTG GAAGCACATACTATAATTCAGCTCTCAAATCCAGACTGAGCATCAGCAAGGACA ACTCCAAGAGCCAAGTTTTCTTAAAAATGAACAGTCTGCAAACTGATGACACAG CCATGTACTACTGTGCCAAAGAGAAACGGAGGGGGTATTACTATGCTATGGACT ACTGGGGTCAAGGAACCTCAGTCACCGTCTCCTCA (SEQ ID NO: 7) or an antigen binding fragment thereof or an equivalent of each thereof.

In some embodiments, the heavy chain variable region comprises, or alternatively consists essentially of, or yet further consists of, the polypeptide encoded by the below noted polynucleotide sequences: GAGGTACAGCTGCAGCAGTCTGGACCTGAGCTGGTGAAGCCTGGGGCTTCAGTG AAGATATCCTGCAAGGCTTCTGGTTACTCATTCACTGGCTACTACATGCACTGGG TGAAGCAAAGCCATGTAAAGAGCCTTGAGTGGATTGGACGTATTAATCCTTACA ATGGTGCTACTAGCTACAACCAGAATTTCAAGGACAAGGCCAGCTTGACTGTAG ATAAGTCCTCCAGCACAGCCTACATGGAGCTCCACAGCCTGACATCTGAGGACTC TGCAGTCTATTACTGTGCAAGACTAGACCGGGGGGACTACTGGGGTCAAGGAAC CTCAGTCACCGTCTCCTCA (SEQ ID NO: 15) or an antigen binding fragment thereof or an equivalent of each thereof.

In some embodiments, the heavy chain variable region comprises, or alternatively consists essentially of, or yet further consists of, the polypeptide encoded by the below noted polynucleotide sequences: CAGGTCCAACTGCAGCAGTCAGGAGCTGAACTGGTCAAGACTGGGGCCTCAGTG AAGATGTCCTGCAAGGCTTCTGGCTACACCTTTACCAGATACTGGATGCACTGGG TAAAACAGAGGCCTGGACAGGCTCTGGAATGGATTGGCGCTATTTATCCTGGAA ATAGTGATACTAGCTACTACCAGAAGTTCAAGGGCAAGGCCAAACTGACTGCAG TCACATCTGCCAGCACTGCCTACATGGAGCTCAGCAGCCTGACATCTGAGGACTC TGCCGTCTATTACTGTGCAAGAGGGGAGGAAATAGGGGTACGACGCTGGTTTGC TTACTGGGGCCAAGGGACTCTGGTCACTGTCTCTGCA (SEQ ID NO: 23) or an antigen binding fragment thereof or an equivalent of each thereof.

In some embodiments, the heavy chain variable region comprises, or alternatively consists essentially of, or yet further consists of, the polypeptide encoded by the below noted polynucleotide sequences: CAGGTGCAGC TGCAGGAGTC CGGCCCAGGA CTGGTGAAGC CTTCGGAGAC CCTGTCCCTC ACCTGCGCTG TCTCTGGTTA CTCCATCAGC AGTGGTTACT ACTGGGGCTG GATTCGGCAG CCCCCAGGGA AGGGGCTGGA GTGGATTGGG AGTATCTATC ATAGTGGGAG CACCTACTAC AACCCGTCCC TCAAGAGTCG AGTCACCATA TCAGTAGACA CGTCCAAGAA CCAGTTCTCC CTGAAGCTGA GCTCTGTGAC CGCCGCAGAC ACGGCCGTGT ATTACTGTGC AAGAGGGAAG TGGTCGAAGT TTGACTATTG GGGCCAAGGC ACCCTGGTCA CCGTCTCTTC A (SEQ ID NO: 31) or an antigen binding fragment thereof or an equivalent of each thereof.

In some embodiments, the light chain variable region comprises a CDRL1 sequence comprising, or alternatively consisting essentially of, or yet further consisting of, an amino acid sequence beginning with any one of the following sequences: (i) SSVSSSY (SEQ ID NO: 4), (ii) ENVVTY (SEQ ID NO: 12), (iii) QSISNY (SEQ ID NO: 20), (iv) QGDSLRSYYAS (SEQ ID NO: 28), or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises a CDRL2 sequence comprising, or alternatively consisting essentially of, or yet further consisting of, an amino acid sequence beginning with any one of the following sequences: (i) STS (SEQ ID NO: 5), (ii) GAS (SEQ ID NO: 13), (iii) YAS (SEQ ID NO: 21), (iv) GKNNRPS (SEQ ID NO: 29), or equivalents of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises a CDRL3 sequence comprising, or alternatively consisting essentially of, or yet further consisting of, an amino acid sequence beginning with any one of the following sequences: (i) QQYSGYPLT (SEQ ID NO: 6), (ii) GQGYSYPYT (SEQ ID NO: 14), (iii) QQSNSWPLT (SEQ ID NO: 22), (iv) NSRDSSGNHVV (SEQ ID NO: 30), or equivalent of each thereof, followed by an additional 50 amino acids, or alternatively about 40 amino acids, or alternatively about 30 amino acids, or alternatively about 20 amino acids, or alternatively about 10 amino acids, or alternatively about 5 amino acids, or alternatively about 4, or 3, or 2 or 1 amino acids at the carboxy-terminus.

In some embodiments, the light chain variable region comprises, or alternatively consists essentially of, or yet further consists of, the polypeptide encoded by the polynucleotide sequence: GGAGAAAATGTGCTCACCCAGTCTCCAGCAATCATGTCTGCATCTCCAGGGGAA AAGGTCACCATGACCTGCAGGGCCAGCTCAAGTGTAAGTTCCAGTTACTTGCACT GGTACCAGCAGAAGTCAGGTGCCTCCCCCAAACTCTGGATTTATAGCACATCCAA CTTGGCTTCTGGAGTCCCTGCTCGCTTCAGTGGCAGTGGGTCTGGGACCTCTTACT CTCTCACAATCAGCAGTGTGGAGGCTGAAGATGCTGCCACTTATTACTGCCAGCA GTACAGTGGTTACCCACTCACGTTCGGAGGGGGGACCAAGCTGGAAATAAAA (SEQ ID NO: 8) or an antigen binding fragment thereof or an equivalent of each thereof.

In some embodiments, the light chain variable region comprises, or alternatively consists essentially of, or yet further consists of, the polypeptide encoded by the polynucleotide sequence: AACATTGTAATGACCCAATCTCCCAAATCCATGTCCATGTCAGTAGGAGAGAGG GTCACCTTGACCTGCAAGGCCAGTGAGAATGTGGTTACTTATGTTTCCTGGTATC AACAGAAACCAGAGCAGTCTCCTAAACTGCTGATATACGGGGCATCCAACCGGT ACACTGGGGTCCCCGATCGCTTCACAGGCAGTGGATCTGCAACAGATTTCACTCT GACCATCAGCAGTGTGCAGGCTGAAGACCTTGCAGATTATCACTGTGGACAGGG TTACAGCTATCCGTACACGTTCGGAGGGGGGACAAAGTTGGAAATAAAACGTACG (SEQ ID NO: 16) or an antigen binding fragment thereof or an equivalent of each thereof.

In some embodiments, the light chain variable region comprises, or alternatively consists essentially of, or yet further consists of, the polypeptide encoded by the polynucleotide sequence: GATATTGTGCTAACTCAGTCTCCAGCCACCCTGTCTGTGACTCCAGGAGATAGAG TCAGTCTTTCCTGCAGGGCCAGGCAAAGTATTAGCAACTACCTACACTGGTATCA ACAAAAATCACATGAGTCTCCAAGGCTTCTCATCAAGTATGCTTCCCAGTCCATC TCTGGCATCCCCTCCAGGTTCAGTGGCAGTGGATCAGGGACAGATTTCACTCTCA GTATCAACAGTGTGGAGACTGAAGATTTTGGAATGTATTTCTGTCAACAGAGTAA CAGCTGGCCGCTCACGTTCGGTGCTGGGACCAAGCTGGAAATAAAA (SEQ ID NO: 24) or an antigen binding fragment thereof or an equivalent of each thereof.

In some embodiments, the light chain variable region comprises, or alternatively consists essentially of, or yet further consists of, the polypeptide encoded by the polynucleotide sequence: TCCTCTGAGC TGACTCAGGA CCCTGCTGTG TCTGTGGCCT TGGGACAGAC AGTCAGGATC ACATGCCAAG GAGACAGCCT CAGAAGCTAT TATGCAAGCT GGTACCAGCA GAAGCCAGGA CAGGCCCCTG TACTTGTCAT CTATGGTAAA AACAACCGGC CCTCAGGGAT TCCAGACCGA TTCTCTGGCT CCAGCTCAGG AAACACAGCT TCCTTGACCA TCACTGGGGC TCAGGCGGAA GATGAGGCTG ACTATTACTG TAACTCCCGG GACAGCAGTG GTAACCATGT GGTATTCGGC GGAGGGACCA AGCTGACCGT CCTA (SEQ ID NO: 32) or an antigen binding fragment thereof or an equivalent of each thereof.

In another aspect of the present disclosure, the antigen binding domain of a TNT antibody includes one or more of the following characteristics:

(a) the light chain immunoglobulin variable domain sequence comprises one or more CDRs that are at least 80% identical to a CDR of a light chain variable domain of any of the disclosed light chain sequences;

(b) the heavy chain immunoglobulin variable domain sequence comprises one or more CDRs that are at least 80% identical to a CDR of a heavy chain variable domain of any of the disclosed heavy chain sequences;

(c) the light chain immunoglobulin variable domain sequence is at least 80% identical to a light chain variable domain of any of the disclosed light chain sequences;

(d) the HC immunoglobulin variable domain sequence is at least 80% identical to a heavy chain variable domain of any of the disclosed light chain sequences; and

(e) the antibody binds an epitope that overlaps with an epitope bound by any of the disclosed sequences.

Additional examples of equivalents include peptide having at least 85%, or alternatively at least 90%, or alternatively at least 95%, or alternatively at least 97% amino acid identity to the peptide or a polypeptide that is encoded by a polynucleotide that hybridizes under conditions of high stringency to the complement of a polynucleotide encoding the antigen binding domain, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

Exemplary antigen binding domains can comprise one or more of the below noted peptides, and in one aspect may comprise the all three CDRs of the noted HC and LC for a particular antigen disclosed in Table 1 and Table 2, respectively.

TABLE 1

| ANTI-TNT | | | | | | |
|---|---|---|---|---|---|---|
| ANTIBODY | CDRH1 | CDRH2 | CDRH3 | CDRL1 | CDRL2 | CDRL3 |
| TNT-1 | SEQ ID | SEQ ID | SEQ ID | SEQ ID | SEQ ID | SEQ ID |
| | NO: 1 | NO: 2 | NO: 3 | NO: 4 | NO: 5 | NO: 6 |
| TNT-2 | SEQ ID | SEQ ID | SEQ ID | SEQ ID | SEQ ID | SEQ ID |
| | NO: 9 | NO: 10 | NO: 11 | NO: 12 | NO: 13 | NO: 14 |
| TNT-3 | SEQ ID | SEQ ID | SEQ ID | SEQ ID | SEQ ID | SEQ ID |
| | NO: 17 | NO: 18 | NO: 19 | NO: 20 | NO: 21 | NO: 22 |
| NHS76 | SEQ ID | SEQ ID | SEQ ID | SEQ ID | SEQ ID | SEQ ID |
| | NO: 25 | NO: 26 | NO: 27 | NO: 28 | NO: 29 | NO: 30 |

TABLE 2

| ANTI-TNT | Heavy Chain Variable | Light Chain Variable |
|---|---|---|
| ANTIBODY | Region | Region |
| TNT-1 | SEQ ID NO: 7 | SEQ ID NO: 8 |
| TNT-2 | SEQ ID NO: 15 | SEQ ID NO: 16 |
| TNT-3 | SEQ ID NO: 23 | SEQ ID NO: 24 |
| NHS76 | SEQ ID NO: 31 | SEQ ID NO: 32 |

In one aspect, the present disclosure provides the antigen binding domain of an antibody that is at least 80%, or alternatively 85%, or alternatively 90%, or alternatively 95%, or alternatively at least 97%, identical to an antibody selected from the group consisting of TNT-1, TNT-2, TNT-3, and NHS76. Additional examples of equivalents include polypeptide that is encoded by a polynucleotide that hybridizes under conditions of high stringency to the complement of a polynucleotide encoding the antigen binding domain, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

In some aspects of the antibodies provided herein, the HC variable domain sequence comprises a variable domain sequence of TNT-1 and the LC variable domain sequence comprises a variable domain sequence of TNT-1.

In one aspect, the present disclosure provides the antigen binding domain of an antibody comprising the CDRs of TNT-1. In one aspect, the present disclosure provides the antigen binding domain of antibody that is at least 85%, or alternatively 80%, or alternatively 85%, or alternatively 90%, or alternatively 95%, or alternatively at least 97% identical to the CDRs of TNT-1, or a polypeptide that is encoded by a polynucleotide that hybridizes under conditions of high stringency to the complement of a polynucleotide encoding the CDRs of TNT, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

In some aspects of the antibodies provided herein, the HC variable domain sequence comprises a variable domain sequence of TNT-2 and the LC variable domain sequence comprises a variable domain sequence of TNT-2.

In one aspect, the present disclosure provides the antigen binding domain of an antibody comprising the CDRs of TNT-2. In one aspect, the present disclosure provides the antigen binding domain of antibody that is at least 85%, or alternatively 80%, or alternatively 85%, or alternatively 90%, or alternatively 95%, or alternatively at least 97% identical to the CDRs of TNT-2, or a polypeptide that is encoded by a polynucleotide that hybridizes under conditions of high stringency to the complement of a polynucleotide encoding the CDRs of TNT-2, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

In some aspects of the antibodies provided herein, the HC variable domain sequence comprises a variable domain sequence of TNT-3 and the LC variable domain sequence comprises a variable domain sequence of TNT-3.

In one aspect, the present disclosure provides the antigen binding domain of an antibody comprising the CDRs of TNT-3. In one aspect, the present disclosure provides the antigen binding domain of antibody that is at least 85%, or alternatively 85%, or alternatively 90%, or alternatively 95%, or alternatively at least 97% identical to the CDRs of TNT-3, or a polypeptide that is encoded by a polynucleotide that hybridizes under conditions of high stringency to the complement of a polynucleotide encoding the CDRs of TNT-3, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

In some aspects of the antibodies provided herein, the HC variable domain sequence comprises a variable domain sequence of NHS76 and the LC variable domain sequence comprises a variable domain sequence of NHS76 or an equivalent of each thereof.

In one aspect, the present disclosure provides the antigen binding domain of an antibody comprising the CDRs of NHS76. In one aspect, the present disclosure provides the antigen binding domain of antibody that is at least 80% identical to NHS76, or at least 85%, or alternatively 85%, or alternatively 90%, or alternatively 95%, or alternatively at least 97% identical to the CDRs of NHS76, or a polypeptide that is encoded by a polynucleotide that hybridizes under conditions of high stringency to the complement of a polynucleotide encoding the CDRs of NHS76, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

Transmembrane Domain.

The transmembrane domain may be derived either from a natural or from a synthetic source. Where the source is natural, the domain may be derived from any membrane-bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Cytoplasmic Domain.

The cytoplasmic domain or intracellular signaling domain of the CAR is responsible for activation of at least one of the traditional effector functions of an immune cell in which a CAR has been placed. The intracellular signaling domain refers to a portion of a protein which transduces the effector function signal and directs the immune cell to perform its specific function. An entire signaling domain or a truncated portion thereof may be used so long as the truncated portion is sufficient to transduce the effector function signal. Cytoplasmic sequences of the TCR and co-receptors as well as derivatives or variants thereof can function as intracellular signaling domains for use in a CAR. Intracellular signaling domains of particular use in this disclosure may be derived from FcR, TCR, CD3, CDS, CD22, CD79a, CD79b, CD66d. Since signals generated through the TCR are alone insufficient for full activation of a T cell, a secondary or co-stimulatory signal may also be required. Thus, the intracellular region of a co-stimulatory signaling molecule, including but not limited CD27, CD28, 4-IBB (CD 137), OX40, CD30, CD40, PD-1, ICOS, lymphocyte function-associated antigen-1 (LFA-1), CD2, CD7, LIGHT, NKG2C, B7-H3, or a ligand that specifically binds with CD83, to may also be included in the cytoplasmic domain of the CAR.

In some embodiments, the cell activation moiety of the chimeric antigen receptor is a T-cell signaling domain comprising, or alternatively consisting essentially of, or yet further consisting of, one or more proteins or fragments thereof selected from the group consisting of CD8 protein, CD28 protein, 4-1BB protein, and CD3-zeta protein.

In specific embodiments, the CAR comprises, or alternatively consists essentially thereof, or yet consists of an antigen binding domain of an TNT antibody, a CD8 α hinge domain, a CD8 α transmembrane domain, a costimulatory signaling region, and a CD3 zeta signaling domain. In further embodiments, the costimulatory signaling region comprises either or both a CD28 costimulatory signaling region and a 4-1BB costimulatory signaling region.

In some embodiments, the CAR can further comprise a detectable marker or purification marker. In another aspect, the CARs as described herein are contained in a composition, e.g., a pharmaceutically acceptable carrier for diagnosis or therapy.

II. Process for Preparing TNT Antibodies

Antibodies for use in this disclosure can be purchased or prepared using methods known in the art and briefly described herein. Their manufacture and uses are well known and disclosed in, for example, Harlow, E. and Lane, D., Antibodies: A Laboratory Manual, Cold Spring Harbor Laboratory Press, Cold Spring Harbor, N.Y., 1999. The antibodies may be generated using standard methods known in the art. Examples of antibodies include (but are not limited to) monoclonal, single chain, and functional fragments of antibodies.

Antibodies may be produced in a range of hosts, for example goats, rabbits, rats, mice, humans, and others. They may be immunized by injection with a target antigen or a fragment or oligopeptide thereof which has immunogenic properties, such as a C-terminal fragment a TNT relevant antigen or an isolated polypeptide. Depending on the host species, various adjuvants may be added and used to increase an immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (Bacille Calmette-Guerin) and Corynebacterium parvum are particularly useful. This disclosure also provides the isolated polypeptide and an adjuvant.

In certain aspects, the antibodies of the present disclosure are polyclonal, i.e., a mixture of plural types of TNT antibodies having different amino acid sequences. In one aspect, the polyclonal antibody comprises a mixture of plural types of TNT antibodies having different CDRs. As such, a mixture of cells which produce different antibodies is cultured, and an antibody purified from the resulting culture can be used (see WO 2004/061104).

Monoclonal Antibody Production.

Monoclonal antibodies to TNT relevant antigen may be prepared using any technique which provides for the production of antibody molecules by continuous cell lines in culture. Such techniques include, but are not limited to, the hybridoma technique (see, e.g., Kohler & Milstein, Nature 256: 495-497 (1975)); the trioma technique; the human B-cell hybridoma technique (see, e.g., Kozbor et al., Immunol. Today 4: 72 (1983)) and the EBV hybridoma technique to produce human monoclonal antibodies (see, e.g., Cole et al., in: MONOCLONAL ANTIBODIES AND CANCER THERAPY, Alan R. Liss, Inc., pp. 77-96 (1985)). Human monoclonal antibodies can be utilized in the practice of the present technology and can be produced by using human hybridomas (see, e.g., Cote et al., Proc. Natl. Acad. Sci. 80: 2026-2030 (1983)) or by transforming human B-cells with Epstein Barr Virus in vitro (see, e.g., Cole et al., in: MONOCLONAL ANTIBODIES AND CANCER THERAPY, Alan R. Liss, Inc., pp. 77-96 (1985)). For example, a population of nucleic acids that encode regions of antibodies can be isolated. PCR utilizing primers derived from sequences encoding conserved regions of antibodies is used to amplify sequences encoding portions of antibodies from the population and then reconstruct DNAs encoding antibodies or fragments thereof, such as variable domains, from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the TNT relevant antigen polypeptide. Alternatively, hybridomas expressing TNT monoclonal antibodies can be prepared by immunizing a subject, e.g., with an isolated polypeptide comprising, or alternatively consisting essentially of, or yet further consisting of, the amino acid sequence of the TNT relevant antigen or a fragment thereof, and then isolating hybridomas from the subject's spleen using routine methods. See, e.g., Milstein et al., (Galfre and Milstein, Methods Enzymol 73: 3-46 (1981)). Screening the hybridomas using standard methods will produce monoclonal antibodies of varying specificity (i.e., for different epitopes) and affinity. A selected monoclonal antibody with the desired properties, e.g., TNT relevant antigen binding, can be (i) used as expressed by the hybridoma, (ii) bound to a molecule such as polyethylene glycol (PEG) to alter its properties, or (iii) a cDNA encoding the monoclonal antibody can be isolated, sequenced and manipulated in various ways. In one aspect, the TNT monoclonal antibody is produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, N.Y., 349 (1988); Hammerling et al., Monoclonal Antibodies And T-Cell Hybridomas, 563-681 (1981).

Phage Display Technique.

As noted above, the antibodies of the present disclosure can be produced through the application of recombinant DNA and phage display technology. For example, TNT antibodies, can be prepared using various phage display methods known in the art. In phage display methods, functional antibody domains are displayed on the surface of a phage particle which carries polynucleotide sequences encoding them. Phage with a desired binding property is selected from a repertoire or combinatorial antibody library (e.g., human or murine) by selecting directly with an antigen, typically an antigen bound or captured to a solid surface or bead. Phage used in these methods are typically filamentous phage including fd and M13 with Fab, Fᵥ or disulfide stabilized Fᵥ antibody domains are recombinantly fused to either the phage gene III or gene VIII protein. In addition, methods can be adapted for the construction of Fab expression libraries (see, e.g., Huse et al., Science 246: 1275-1281, 1989) to allow rapid and effective identification of monoclonal Fab fragments with the desired specificity for a TNT relevant antigen polypeptide, e.g., a polypeptide or derivatives, fragments, analogs or homologs thereof. Other examples of phage display methods that can be used to make the isolated antibodies of the present disclosure include those disclosed in Huston et al., Proc. Natl. Acad. Sci. U.S.A., 85: 5879-5883 (1988); Chaudhary et al., Proc. Natl. Acad. Sci. U.S.A., 87: 1066-1070 (1990); Brinkman et al., J. Immunol. Methods 182: 41-50 (1995); Ames et al., J. Immunol. Methods 184: 177-186 (1995); Kettleborough et al., Eur. J. Immunol. 24: 952-958 (1994); Persic et al., Gene 187: 9-18 (1997); Burton et al., Advances in Immunology 57: 191-280 (1994); PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743.

Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12: 864-869 (1992); Sawai et al., AJRI 34: 26-34 (1995); and Better et al., Science 240: 1041-1043 (1988).

Generally, hybrid antibodies or hybrid antibody fragments that are cloned into a display vector can be selected against the appropriate antigen in order to identify variants that maintained good binding activity, because the antibody or antibody fragment will be present on the surface of the phage or phagemid particle. See, e.g., Barbas III et al., Phage Display, A Laboratory Manual (Cold Spring Harbor Laboratory Press, Cold Spring Harbor, N.Y., 2001). However, other vector formats could be used for this process, such as cloning the antibody fragment library into a lytic phage vector (modified T7 or Lambda Zap systems) for selection and/or screening.

Alternate Methods of Antibody Production.

Antibodies may also be produced by inducing in vivo production in the lymphocyte population or by screening recombinant immunoglobulin libraries or panels of highly specific binding reagents (Orlandi et al., PNAS 86: 3833-3837 (1989); Winter, G. et al., Nature, 349: 293-299 (1991)).

Alternatively, techniques for the production of single chain antibodies may be used. Single chain antibodies (scFᵥs) comprise a heavy chain variable region and a light chain variable region connected with a linker peptide (typically around 5 to 25 amino acids in length). In the scFᵥ, the variable regions of the heavy chain and the light chain may be derived from the same antibody or different antibodies. scFᵥs may be synthesized using recombinant techniques, for example by expression of a vector encoding the scFᵥ in a host organism such as E. coli. DNA encoding scFᵥ can be obtained by performing amplification using a partial DNA encoding the entire or a desired amino acid sequence of a DNA selected from a DNA encoding the heavy chain or the variable region of the heavy chain of the above-mentioned antibody and a DNA encoding the light chain or the variable region of the light chain thereof as a template, by PCR using a primer pair that defines both ends thereof, and further performing amplification combining a DNA encoding a polypeptide linker portion and a primer pair that defines both ends thereof, so as to ligate both ends of the linker to the heavy chain and the light chain, respectively. An expression vector containing the DNA encoding scFᵥ and a host transformed by the expression vector can be obtained according to conventional methods known in the art.

Antigen binding fragments may also be generated, for example the F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed to allow rapid and easy identification of monoclonal Fab fragments with the desired specificity (Huse et al., Science, 256: 1275-1281 (1989)).

Antibody Modifications.

The antibodies of the present disclosure may be multimerized to increase the affinity for an antigen. The antibody to be multimerized may be one type of antibody or a plurality of antibodies which recognize a plurality of epitopes of the same antigen. As a method of multimerization of the antibody, binding of the IgG CH3 domain to two scFᵥ molecules, binding to streptavidin, introduction of a helix-turn-helix motif and the like can be exemplified.

The antibody compositions disclosed herein may be in the form of a conjugate formed between any of these antibodies and another agent (immunoconjugate). In one aspect, the antibodies disclosed herein are conjugated to radioactive material. In another aspect, the antibodies disclosed herein can be bound to various types of molecules such as polyethylene glycol (PEG).

Antibody Screening.

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between the TNT relevant antigen, or any fragment or oligopeptide thereof and its specific antibody. A two-site, monoclonal-based immunoassay utilizing monoclonal antibodies specific to two non-interfering TNT relevant antigen epitopes may be used, but a competitive binding assay may also be employed (Maddox et al., J. Exp. Med., 158: 1211-1216 (1983)).

Antibody Purification.

The antibodies disclosed herein can be purified to homogeneity. The separation and purification of the antibodies can be performed by employing conventional protein separation and purification methods.

By way of example only, the antibody can be separated and purified by appropriately selecting and combining use of chromatography columns, filters, ultrafiltration, salt precipitation, dialysis, preparative polyacrylamide gel electrophoresis, isoelectric focusing electrophoresis, and the like. Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory (1988).

Examples of chromatography include affinity chromatography, ion exchange chromatography, hydrophobic chromatography, gel filtration chromatography, reverse phase chromatography, and adsorption chromatography. In one aspect, chromatography can be performed by employing liquid chromatography such as HPLC or FPLC.

In one aspect, a Protein A column or a Protein G column may be used in affinity chromatography. Other exemplary columns include a Protein A column, Hyper D, POROS, Sepharose F. F. (Pharmacia) and the like.

III. Isolated Nucleic Acids and Processes for Preparing CARs

Aspects of the present disclosure relate to an isolated cell comprising a TNT CAR and methods of producing such cells. The cell is a prokaryotic or a eukaryotic cell. In one aspect, the cell is a T cell, B cell, or an NK cell. The eukaryotic cell can be from any preferred species, e.g., an animal cell, a mammalian cell such as a human, a feline or a canine cell.

In specific embodiments, the isolated cell comprises, or alternatively consists essentially of, or yet further consists of an exogenous CAR comprising, or alternatively consisting essentially of, or yet further consisting of, an antigen binding domain of an TNT antibody, a CD8 α hinge domain, a CD8 α transmembrane domain, a CD28 costimulatory signaling region and/or a 4-1BB costimulatory signaling region, and a CD3 zeta signaling domain. In certain embodiments, the isolated cell is a T-cell, e.g., an animal T-cell, a mammalian T-cell, a feline T-cell, a canine T-cell or a human T-cell. In certain embodiments, the isolated cell is an NK-cell, e.g., an animal NK-cell, a mammalian NK-cell, a feline NK-cell, a canine NK-cell or a human NK-cell.

In certain embodiments, methods of producing TNT CAR expressing cells are disclosed the method comprising, or alternatively consisting essentially of or yet further consisting of transducing a population of isolated cells with a nucleic acid sequence encoding a TNT CAR. In a further aspect, a subpopulation of cells that have been successfully transduced with said nucleic acid sequence is selected. In some embodiments, the isolated cells are T-cells, an animal T-cell, a mammalian T-cell, a feline T-cell, a canine T-cell or a human T-cell, thereby producing TNT CAR T-cells. In certain embodiments, the isolated cell is an NK-cell, e.g., an animal NK-cell, a mammalian NK-cell, a feline NK-cell, a canine NK-cell or a human NK-cell, thereby producing TNT CAR NK-cells. In some embodiments, the isolated cells are B-cells, an animal B-cell, a mammalian B-cell, a feline B-cell, a canine B-cell or a human B-cell, thereby producing TNT CAR B-cells.

Sources of Isolated Cells.

Prior to expansion and genetic modification of the cells disclosed herein, cells may be obtained from a subject—for instance, in embodiments involving autologous therapy—or a commercially available culture.

Cells can be obtained from a number of sources in a subject, including peripheral blood mononuclear cells, bone marrow, lymph node tissue, cord blood, thymus tissue, tissue from a site of infection, ascites, pleural effusion, spleen tissue, and tumors.

Methods of isolating relevant cells are well known in the art and can be readily adapted to the present application; an exemplary method is described in the examples below. Isolation methods for use in relation to this disclosure include, but are not limited to Life Technologies Dynabeads® system; STEMcell Technologies EasySep™, RoboSep™ RosetteSep™, SepMate™; Miltenyi Biotec MACS™ cell separation kits, and other commercially available cell separation and isolation kits. Particular subpopulations of immune cells may be isolated through the use of beads or other binding agents available in such kits specific to unique cell surface markers. For example, MACS™ CD4+ and CD8+ MicroBeads may be used to isolate CD4+ and CD8+ T-cells.

Alternatively, cells may be obtained through commercially available cell cultures, including but not limited to, for T-cells, lines BCL2 (AAA) Jurkat (ATCC® CRL-2902™), BCL2 (S70A) Jurkat (ATCC® CRL-2900™), BCL2 (S87A) Jurkat (ATCC® CRL-2901™), BCL2 Jurkat (ATCC® CRL-2899™), Neo Jurkat (ATCC® CRL-2898™); for B cells, lines AHH-1 (ATCC® CRL-8146™), BC-1 (ATCC® CRL-2230™), BC-2 (ATCC® CRL-2231™), BC-3 (ATCC® CRL-2277™), CA46 (ATCC® CRL-1648™), DG-75 [D.G.-75] (ATCC® CRL-2625™), DS-1 (ATCC® CRL-11102™), EB-3 [EB3] (ATCC® CCL-85™), Z-138 (ATCC #CRL-3001), DB (ATCC CRL-2289), Toledo (ATCC CRL-2631), Pfiffer (ATCC CRL-2632), SR (ATCC CRL-2262), JM-1 (ATCC CRL-10421), NFS-5 C-1 (ATCC CRL-1693); NFS-70 C10 (ATCC CRL-1694), NFS-25 C-3 (ATCC CRL-1695), and SUP-B15 (ATCC CRL-1929); and, for NK cells, lines NK-92 (ATCC® CRL-2407™), NK-92MI (ATCC® CRL-2408™). Further examples include but are not limited to mature T-cell lines, e.g., Deglis, EBT-8, HPB-MLp-W, HUT 78, HUT 102, Karpas 384, Ki 225, My-La, Se-Ax, SKW-3, SMZ-1 and T34; immature T-cell lines, e.g., ALL-SIL, Be13, CCRF-CEM, CML-T1, DND-41, DU.528, EU-9, HD-Mar, HPB-ALL, H-SB2, HT-1, JK-T1, Jurkat, Karpas 45, KE-37, KOPT-K1, K-T1, L-KAW, Loucy, MAT, MOLT-1, MOLT 3, MOLT-4, MOLT 13, MOLT-16, MT-1, MT-ALL, P12/Ichikawa, Peer, PER0117, PER-255, PF-382, PFI-285, RPMI-8402, ST-4, SUP-T1 to T14, TALL-1, TALL-101, TALL-103/2, TALL-104, TALL-105, TALL-106, TALL-107, TALL-197, TK-6, TLBR-1, -2, -3, and -4, CCRF-HSB-2 (CCL-120.1), J.RT3-T3.5 (ATCC TIB-153), J45.01 (ATCC CRL-1990), J.CaM1.6 (ATCC CRL-2063), RS4; 11 (ATCC CRL-1873), CCRF-CEM (ATCC CRM-CCL-119); cutaneous T-cell lymphoma lines, e.g., HuT78 (ATCC CRM-TIB-161), MJ[G11] (ATCC CRL-8294), HuT102 (ATCC TIB-162); B-cell lines derived from anaplastic and large cell lymphomas, e.g., DEL, DL-40, FE-PD, JB6, Karpas 299, Ki-JK, Mac-2A Ply1, SR-786, SU-DHL-1, -2, -4, -5, -6, -7, -8, -9, -10, and -16, DOHH-2, NU-DHL-1, U-937, Granda 519, USC-DHL-1, RL; Hodgkin's lymphomas, e.g., DEV, HD-70, HDLM-2, HD-MyZ, HKB-1, KM-H2, L 428, L 540, L1236, SBH-1, SUP-HD1, and SU/RH-HD-1; and NK lines such as HANK1, KHYG-1, NKL, NK-YS, NOI-90, and YT. Null leukemia cell lines, including but not limited to REH, NALL-1, KM-3, L92-221, are a another commercially available source of immune cells, as are cell lines derived from other leukemias and lymphomas, such as K562 erythroleukemia, THP-1 monocytic leukemia, U937 lymphoma, HEL erythroleukemia, HL60 leukemia, HMC-1 leukemia, KG-1 leukemia, U266 myeloma. Non-limiting exemplary sources for such commercially available cell lines include the American Type Culture Collection, or ATCC, (http://www.atcc.org/) and the German Collection of Microorganisms and Cell Cultures (https://www.dsmz.de/).

Construction of NFAT Linked Polynucleotides.

Some aspects of the present disclosure relate to an isolated nucleic acid comprising an NFAT regulatory polynucleotide operatively linked to a polynucleotide encoding an immunoregulatory molecule. Techniques for preparing such linked polynucleotides are described in U.S. Pat. No. 8,556,882.

In some embodiments, the NFAT regulatory polynucleotide comprises, or alternatively consists essentially thereof, or yet further consists of an NFAT responsive element. Examples of NFAT responsive elements include but are not limited to NFAT1, NFAT2, NFAT3, NFAT4, and/or a complement or any equivalents thereof. In some embodiments, the NFAT regulatory polynucleotide comprises, or alternatively consists essentially of, or yet further consists of one or more binding motifs to which NFAT may bind. In some embodiments, the NFAT regulatory polynucleotide comprises one or more repetitions of the same binding motif and/or NFAT responsive element; in some embodiments, the NFAT regulatory polynucleotide comprises one or more different NFAT binding motifs and/or NFAT responsive elements. In certain embodiments, the NFAT regulatory polynucleotide comprises, or alternatively consists essentially of, or yet further consists of between 1 and 15, preferably between 2 and 10, still more preferably between 3 and 9, still more preferably 6 NFAT binding motifs and/or NFAT responsive elements. In a specific embodiment, the NFAT regulatory polynucleotide comprises, or alternatively consists essentially of, or yet further consists of 6 repeats of the same NFAT binding motif or NFAT responsive element.

In some embodiments, the NFAT regulatory polynucleotide comprises, or alternatively consists essentially thereof, or yet further consists of the sequence: AGGAAAAAC (SEQ ID NO: 58) or an equivalent thereof.

In some embodiments, the NFAT regulatory polynucleotide comprises, or alternatively consists essentially of, or yet further consists of the sequence: GGAGGAAAAACTGTTTCATACAGAAGGCG (SEQ ID NO: 57) or equivalent thereof.

In certain embodiments, the NFAT regulatory polynucleotide comprises, or alternatively consists essentially thereof, or yet further consists of SEQ ID NO: 57, SEQ ID NO: 58, or an equivalent thereof repeated 6 times.

In some embodiments, the polynucleotide encoding an immunoregulatory molecule comprises, or alternatively consists essentially of, or yet further consists of a sequence encoding the nucleotide sequence of the immunoregulatory molecule, a functional portion or variant thereof.

In some embodiments, the isolated nucleotide comprises, or alternatively consists essentially of, or further consists of a promoter sequence. In certain embodiments, the promoter sequence is the promoter sequence for an immunoregulatory molecule. In further embodiments, the promoter sequence may be that of the same immunoregulatory molecule as encoded by the polynucleotide encoding an immunoregulatory molecule. In alternate embodiments, the promoter sequence is that of a different immunoregulatory molecule.

In certain embodiments, the NFAT regulatory polynucleotide is operatively linked to the polynucleotide encoding the immunoregulatory molecule at the 3′ end. In certain embodiments, the promoter sequence is located between the two polynucleotides. In a yet further aspect, the isolated nucleic acids further comprise a detectable label or a gene to assist with selection of transduced cells, e.g., an antibiotic resistance gene.

Vectors.

CAR cells may be prepared using vectors. Aspects of the present disclosure relate to an isolated nucleic acid sequence encoding (i) a TNT CAR or (ii) a polynucleotide encoding an immunoregulatory molecule and vectors comprising, or alternatively consisting essentially of, or yet further consisting of, an either one or both of these nucleic acids and/or complements and/or equivalents of each thereof.

In some embodiments, the isolated nucleic acid sequence encodes for a CAR comprising, or alternatively consisting essentially of, or yet further consisting of an antigen binding domain of an TNT antibody, a CD8 α hinge domain, a CD8 α transmembrane domain, a CD28 costimulatory signaling region and/or a 4-1BB costimulatory signaling region, and a CD3 zeta signaling domain. In specific embodiments, the isolated nucleic acid sequence comprises, or alternatively consisting essentially thereof, or yet further consisting of, sequences encoding (a) an antigen binding domain of an TNT antibody followed by (b) a CD8 α hinge domain, (c) a CD8 α transmembrane domain followed by (d) a CD28 costimulatory signaling region and/or a 4-1BB costimulatory signaling region followed by (e) a CD3 zeta signaling domain.

In some embodiments, the isolated nucleic acid sequence encodes for a CAR and comprises, or alternatively consists essentially of, or yet further consists of, a Kozak consensus sequence upstream of the sequence encoding the antigen binding domain of the TNT antibody.

In some embodiments, the isolated nucleic acid comprises, or alternatively consists essentially of, or yet further consists of an immunoregulatory molecule. In further embodiments, the immunoregulatory molecule is one or more molecule selected from the group consisting of B7.1, CCL19, CCL21, CD40L, CD137L, GITRL, GM-CSF, IL-12, IL-2, low-toxicity IL-2, IL-15, IL-18, IL-21, LEC, and OX40L.

In some embodiments, the isolated nucleic acid comprises, or alternatively consists essentially of, or yet further consists of a polynucleotide sequence encoding immuneregulatory molecule and comprises, or alternatively consists essentially of, or yet further consists of, an NFTA regulatory polynucleotide operatively linked to the polynucleotide encoding the immunoregulatory molecule that may be generated according to the method disclosed above.

In some embodiments, the isolated nucleic acid comprises a detectable label and/or a polynucleotide conferring antibiotic resistance. In one aspect, the label or polynucleotide are useful to select cells successfully transduced with the isolated nucleic acids.

In some embodiments, the isolated nucleic acid sequence is comprised within a vector. In certain embodiments, the vector is a plasmid. In other embodiments, the vector is a viral vector. Non-limiting examples of such include without limitation a retroviral vector, a lentiviral vector, an adenoviral vector, and an adeno-associated viral vector. In specific embodiments, the vector is a lentiviral vector.

The preparation of exemplary vectors and the generation of CAR expressing cells using said vectors is discussed in detail in the examples below. In summary, the expression of natural or synthetic nucleic acids encoding CARs or immunoregulatory molecules is typically achieved by operably linking a nucleic acid encoding the CAR polypeptide or portions thereof to a promoter, and incorporating the construct into an expression vector. A similar method may be used to construct the isolated nucleic acid sequence comprising a polynucleotide encoding an immunoregulatory molecule. The vectors can be suitable for replication and integration eukaryotes. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

In one aspect, the term “vector” intends a recombinant vector that retains the ability to infect and transduce non-dividing and/or slowly-dividing cells and integrate into the target cell's genome. In several aspects, the vector is derived from or based on a wild-type virus. In further aspects, the vector is derived from or based on a wild-type lentivirus. Examples of such include without limitation, human immunodeficiency virus (HIV), equine infectious anemia virus (EIAV), simian immunodeficiency virus (SIV) and feline immunodeficiency virus (Hy). Alternatively, it is contemplated that other retrovirus can be used as a basis for a vector backbone such murine leukemia virus (MLV). It will be evident that a viral vector according to the disclosure need not be confined to the components of a particular virus. The viral vector may comprise components derived from two or more different viruses, and may also comprise synthetic components. Vector components can be manipulated to obtain desired characteristics, such as target cell specificity.

The recombinant vectors of this disclosure are derived from primates and non-primates. Examples of primate lentiviruses include the human immunodeficiency virus (HIV), the causative agent of human acquired immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anemia virus (EIAV) and the more recently described feline immunodeficiency virus (FIV) and bovine immunodeficiency virus (BIV). Prior art recombinant lentiviral vectors are known in the art, e.g., see U.S. Pat. Nos. 6,924,123; 7,056,699; 7,419,829 and 7,442,551, incorporated herein by reference.

U.S. Pat. No. 6,924,123 discloses that certain retroviral sequence facilitate integration into the target cell genome. This patent teaches that each retroviral genome comprises genes called gag, pol and env which code for virion proteins and enzymes. These genes are flanked at both ends by regions called long terminal repeats (LTRs). The LTRs are responsible for proviral integration, and transcription. They also serve as enhancer-promoter sequences. In other words, the LTRs can control the expression of the viral genes. Encapsidation of the retroviral RNAs occurs by virtue of a psi sequence located at the 5′ end of the viral genome. The LTRs themselves are identical sequences that can be divided into three elements, which are called U3, R and U5. U3 is derived from the sequence unique to the 3′ end of the RNA. R is derived from a sequence repeated at both ends of the RNA, and U5 is derived from the sequence unique to the 5′ end of the RNA. The sizes of the three elements can vary considerably among different retroviruses. For the viral genome. and the site of poly (A) addition (termination) is at the boundary between R and U5 in the right hand side LTR. U3 contains most of the transcriptional control elements of the provirus, which include the promoter and multiple enhancer sequences responsive to cellular and in some cases, viral transcriptional activator proteins.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome.

For the production of viral vector particles, the vector RNA genome is expressed from a DNA construct encoding it, in a host cell. The components of the particles not encoded by the vector genome are provided in trans by additional nucleic acid sequences (the “packaging system”, which usually includes either or both of the gag/pol and env genes) expressed in the host cell. The set of sequences required for the production of the viral vector particles may be introduced into the host cell by transient transfection, or they may be integrated into the host cell genome, or they may be provided in a mixture of ways. The techniques involved are known to those skilled in the art.

Retroviral vectors for use in this disclosure include, but are not limited to Invitrogen's pLenti series versions 4, 6, and 6.2 “ViraPower” system. Manufactured by Lentigen Corp.; pHIV-7-GFP, lab generated and used by the City of Hope Research Institute; “Lenti-X” lentiviral vector, pLVX, manufactured by Clontech; pLKO.1-puro, manufactured by Sigma-Aldrich; pLemiR, manufactured by Open Biosystems; and pLV, lab generated and used by Charité Medical School, Institute of Virology (CBF), Berlin, Germany.

Regardless of the method used to introduce exogenous nucleic acids into a host cell or otherwise expose a cell to the inhibitor of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

Packaging Vector and Cell Lines.

The isolated nucleic acids can be packaged into a retroviral packaging system by using a packaging vector and cell lines. The packaging vector includes, but is not limited to retroviral vector, lentiviral vector, adenoviral vector, and adeno-associated viral vector. The packaging vector contains elements and sequences that facilitate the delivery of genetic materials into cells. For example, the retroviral constructs are packaging vectors comprising at least one retroviral helper DNA sequence derived from a replication-incompetent retroviral genome encoding in trans all virion proteins required to package a replication incompetent retroviral vector, and for producing virion proteins capable of packaging the replication-incompetent retroviral vector at high titer, without the production of replication-competent helper virus. The retroviral DNA sequence lacks the region encoding the native enhancer and/or promoter of the viral 5′ LTR of the virus, and lacks both the psi function sequence responsible for packaging helper genome and the 3′ LTR, but encodes a foreign polyadenylation site, for example the SV40 polyadenylation site, and a foreign enhancer and/or promoter which directs efficient transcription in a cell type where virus production is desired. The retrovirus is a leukemia virus such as a Moloney Murine Leukemia Virus (MMLV), the Human Immunodeficiency Virus (HIV), or the Gibbon Ape Leukemia virus (GALV). The foreign enhancer and promoter may be the human cytomegalovirus (HCMV) immediate early (IE) enhancer and promoter, the enhancer and promoter (U3 region) of the Moloney Murine Sarcoma Virus (MMSV), the U3 region of Rous Sarcoma Virus (RSV), the U3 region of Spleen Focus Forming Virus (SFFV), or the HCMV IE enhancer joined to the native Moloney Murine Leukemia Virus (MMLV) promoter. The retroviral packaging vector may consist of two retroviral helper DNA sequences encoded by plasmid based expression vectors, for example where a first helper sequence contains a cDNA encoding the gag and pol proteins of ecotropic MMLV or GALV and a second helper sequence contains a cDNA encoding the env protein. The Env gene, which determines the host range, may be derived from the genes encoding xenotropic, amphotropic, ecotropic, polytropic (mink focus forming) or 10A1 murine leukemia virus env proteins, or the Gibbon Ape Leukemia Virus (GALV env protein, the Human Immunodeficiency Virus env (gp160) protein, the Vesicular Stomatitus Virus (VSV) G protein, the Human T cell leukemia (HTLV) type I and II env gene products, chimeric envelope gene derived from combinations of one or more of the aforementioned env genes or chimeric envelope genes encoding the cytoplasmic and transmembrane of the aforementioned env gene products and a monoclonal antibody directed against a specific surface molecule on a desired target cell.

In the packaging process, the packaging vectors and retroviral vectors are transiently cotransfected into a first population of mammalian cells that are capable of producing virus, such as human embryonic kidney cells, for example 293 cells (ATCC No. CRL1573, ATCC, Rockville, Md.) to produce high titer recombinant retrovirus-containing supernatants. In another method of the disclosure this transiently transfected first population of cells is then cocultivated with mammalian target cells, for example human lymphocytes, to transduce the target cells with the foreign gene at high efficiencies. In yet another method of the invention the supernatants from the above described transiently transfected first population of cells are incubated with mammalian target cells, for example human lymphocytes or hematopoietic stem cells, to transduce the target cells with the foreign gene at high efficiencies.

In another aspect, the packaging vectors are stably expressed in a first population of mammalian cells that are capable of producing virus, such as human embryonic kidney cells, for example 293 cells. Retroviral or lentiviral vectors are introduced into cells by either cotransfection with a selectable marker or infection with pseudotyped virus. In both cases, the vectors integrate. Alternatively, vectors can be introduced in an episomally maintained plasmid. High titer recombinant retrovirus-containing supernatants are produced.

Activation and Expansion of CAR Cells.

Whether prior to or after genetic modification of the cells to express a desirable CAR, the cells can be activated and expanded using generally known methods such as those described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041 and references such as Lapateva et al. (2014) Crit Rev Oncog 19(1-2):121-32; Tam et al. (2003) Cytotherapy 5(3):259-72; Garcia-Marquez et al. (2014) Cytotherapy 16(11):1537-44. Stimulation with the TNT relevant antigen ex vivo can activate and expand the selected CAR expressing cell subpopulation. Alternatively, the cells may be activated in vivo by interaction with TNT relevant antigen.

In the case of certain immune cells, additional cell populations, soluble ligands and/or cytokines, or stimulating agents may be required to activate and expand cells. The relevant reagents are well known in the art and are selected according to known immunological principles. For instance, soluble CD-40 ligand may be helpful in activating and expanding certain B-cell populations; similarly, irradiated feeder cells may be used in the procedure for activation and expansion of NK cells.

Methods of activating relevant cells are well known in the art and can be readily adapted to the present application; an exemplary method is described in the examples below. Isolation methods for use in relation to this disclosure include, but are not limited to Life Technologies Dynabeads® system activation and expansion kits; BD Biosciences Phosflow™ activation kits, Miltenyi Biotec MACS™ activation/expansion kits, and other commercially available cell kits specific to activation moieties of the relevant cell. Particular subpopulations of immune cells may be activated or expanded through the use of beads or other agents available in such kits. For example, α-CD3/α-CD28 Dynabeads® may be used to activate and expand a population of isolated T-cells.

IV. Methods of Use

Therapeutic Application.

Method aspects of the present disclosure relate to methods for inhibiting the growth of a tumor in a subject in need thereof and/or for treating a cancer patient in need thereof. In some embodiments, the tumor is a solid tumor. IN some embodiments, the cancer is a cancer affecting blood and/or bone marrow. In some embodiments, the tumor or cancer cell expresses or overexpresses a TNT-relevant antigen. In certain embodiments, these methods comprise, or alternatively consist essentially of, or yet further consist of, administering to the subject or patient an effective amount of the isolated cell. In further embodiments, this isolated cell comprises a TNT CAR. In still further embodiments, the isolated cell is a T-cell or an NK cell. In some embodiments, the isolated cell is autologous to the subject or patient being treated. In a further aspect, the tumor expresses TNT relevant antigen and the subject has been selected for the therapy by a diagnostic, such as the one described herein. The subject is an animal, a mammal, a canine, a feline, a bovine, an equine, a murine or a human patient.

The TNT CAR cells as disclosed herein may be administered either alone or in combination with diluents, known anti-cancer therapeutics, and/or with other components such as cytokines or other cell populations that are immunoregulatory. They can be administered as a first line therapy, a second line therapy, a third line therapy, or further therapy. Non-limiting examples of additional therapies include cytoreductive therapy, such as radiation therapy, cryotherapy, or chemotherapy, or biologics. Further non-limiting examples include other relevant cell types, such as unmodified immune cells, modified immune cells comprising vectors expressing one or more immunoregulatory molecules, or CAR cells specific to a different antigen than those disclosed herein. As with the CAR cells of the present disclosure, in some embodiments, these cells may be autologous or allogenic. Appropriate treatment regimens will be determined by the treating physician or veterinarian.

Pharmaceutical compositions of the present disclosure may be administered in a manner appropriate to the disease to be treated or prevented. The quantity and frequency of administration will be determined by such factors as the condition of the patient, and the type and severity of the patient's disease, although appropriate dosages may be determined by clinical trials. In one aspect they are administered directly by direct injection or systemically such as intravenous injection.

Aspects of the disclosure provide an exemplary method for determining if a patient is likely to respond to, or is not likely to respond to, TNT CAR therapy. The method comprises, or alternatively consists essentially thereof, or further consists of determining the presence or absence of necrosis in a tumor sample isolated from the patient and quantitating the amount of necrosis present in a given tumor sample, wherein the presence of the necrosis indicates that the patient is likely to respond to the TNT CAR therapy and the absence of necrosis indicates that the patient is not likely to respond to the TNT therapy. In certain embodiments, the method further comprises, or alternatively consists essentially of, or yet further consists of administering an effective amount of the TNT CAR therapy to the patient that is determined likely to respond to the TNT CAR therapy. The TNT CAR therapy can be autologous or allogenic to the patient and the patient can be subject that suffers from a solid tumor, animal or human.

Techniques of histological staining for necrosis are well known in the art. For example, hematoxylin and eosin stains, also referred to as “H&E staining,” are a common technique for identifying the presence of necrosis in tissues, especially in tumorigenic or cancerous growth. Cytoplasmic H&E staining demonstrates increased eosinophilia, attributable in part to the loss of cytoplasmic RNA and in part to denatured cytoplasmic proteins. In necrotic tissue stains, the cytoplasm often appears “moth eaten” due to enzyme digestion of cytoplasmic organelles. Myelin figures, calcification, and evidence of phagocytosis into other cells are also hallmarks of necrotic tissues that can be detected by histological staining. Necrotic tissues also have specific hallmarks in nuclear staining often demonstrating karyolysis, pyknosis, and karyorrhexis as a result of cell death. Using microscopy and either manual or automated quantitation of such necrotic hallmarks, relevance of TNT CAR therapy may be determined. Alternate means of detecting tumorigenic or cancerous growth or necrotic tissues in general, including but not limited to biomarker-based or imaging-based diagnostics, are also equally relevant to determining whether a patient will respond to TNT CAR therapy, and may be used accordingly.

V. Carriers

Additional aspects of the disclosure relate to compositions comprising, or alternatively consisting essentially of, or yet further consisting of, a carrier and one or more of the products—e.g., a TNT CAR, an isolated cell comprising a TNT CAR, an isolated nucleic acid, a vector, an isolated cell containing the TNT CAR and the immunomodulatory molecule and/or nucleic acids encoding such—described in the embodiments disclosed herein. In further aspects, the composition may additionally comprise an immunoregulatory molecule and/or an isolated nucleic acid comprising an NFAT regulatory polynucleotide and a polynucleotide encoding an immunoregulatory molecule.

In certain embodiments, the immunoregulatory molecule is one or more selected from the group consisting of B7.1, CCL19, CCL21, CD40L, CD137L, GITRL, GM-CSF, IL-12, IL-2, low-toxicity IL-2, IL-15, IL-18, IL-21, LEC, and OX40L.

Briefly, pharmaceutical compositions of the present disclosure including but not limited to any one of the claimed compositions as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for oral, intravenous, topical, enteral, and/or parenteral administration. In certain embodiments, the compositions of the present disclosure are formulated for intravenous administration.

Administration of the cells or compositions can be effected in one dose, continuously or intermittently throughout the course of treatment. Methods of determining the most effective means and dosage of administration are known to those of skill in the art and will vary with the composition used for therapy, the purpose of the therapy and the subject being treated. Single or multiple administrations can be carried out with the dose level and pattern being selected by the treating physician. Suitable dosage formulations and methods of administering the agents are known in the art. In a further aspect, the cells and composition of the disclosure can be administered in combination with other treatments.

The cells and populations of cell are administered to the host using methods known in the art and described, for example, in PCT/US2011/064191. This administration of the cells or compositions of the disclosure can be done to generate an animal model of the desired disease, disorder, or condition for experimental and screening assays.

VI. Kits

As set forth herein, the present disclosure provides methods for producing and administering TNT CAR cells. In one particular aspect, the present disclosure provides kits for performing these methods as well as instructions for carrying out the methods of the present disclosure such as collecting cells and/or tissues, and/or performing the screen/transduction/etc., and/or analyzing the results.

In one aspect the kit comprises, or alternatively consists essentially of, or yet further consists of, any one of the isolated nucleic acids disclosed herein and/or a vector comprising said nucleic acid and/or isolated allogenic cells, preferably T cells or NK cells, and/or instructions on the procuring of autologous cells from a patient. Such a kit may also comprise, or alternatively consist essentially of, or yet further comprise media and other reagents appropriate for the transduction and/or selection and/or activation and/or expansion of TNT CAR expressing cells, such as those disclosed herein.

In one aspect the kit comprises, or alternatively consists essentially of, or yet further consists of, an isolated CAR expressing cell or population thereof. In some embodiments, the cells of this kit may require activation and/or expansion prior to administration to a subject in need thereof. In further embodiments, the kit may further comprise, or consist essentially thereof, media and reagents, such as those covered in the disclosure above, to activate and/or expand the isolated CAR expressing cell. In some embodiments, the cell is to be used for TNT CAR therapy. In further embodiments, the kit comprises instructions on the administration of the isolated cell to a patient in need of TNT CAR therapy.

The kits of his disclosure can also comprise, e.g., a buffering agent, a preservative or a protein-stabilizing agent. The kits can further comprise components necessary for detecting the detectable-label, e.g., an enzyme or a substrate. The kits can also contain a control sample or a series of control samples, which can be assayed and compared to the test sample. Each component of a kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

The following examples are illustrative of procedures which can be used in various instances in carrying the disclosure into effect.

### Example 1—LEC Bioactivity

The bioactivity of the LEC fusion protein was demonstrated in vitro by measuring the migration of target cells in a 96-well microchemotaxis chamber (Neuroprobe, Gaithersburg, Md.) as described in the manufacturer's protocol (Li, J. et al. (2003) J Immunother. 26: 320-331; Li, J. et al. (2003) Cancer Res. 63: 8384-8392). Briefly, LEC/chTNT-3, recombinant human LEC, or parental chTNT-3 were serially diluted from 0.39 nM to 50 nM in binding medium (RPMI 1640 with 1% BSA and 25 mmol/L HEPES) and placed in the lower chamber of the microchemotaxis apparatus. One hundred μL binding medium containing 10⁵ THP-1 human monocytic cells were then added to the upper chamber and after 1.5 h of incubation in a humidified 5% CO₂, 37° C. incubator, the percentage of migrated cells was calculated to determine the migration index (average number of cells exposed to chemokine and fusion protein divided by the average number of cells exposed to binding media). All assays were performed in triplicate. As shown in FIG. 5, free human recombinant LEC and the fusion protein induced THP-1 cell migration. The migration of THP-1 cells exposed to the fusion protein was dose dependent starting at a concentration as low as 1.6 nM and peaking at concentration of 12.5 nM. Free human recombinant LEC peaked at a higher concentration of about 25 nM in this assay. THP-1 cells exposed to the parental antibody (chTNT-3) did not show any migration verifying the biologic activity of the LEC moiety of the fusion protein.

### Example 2—Generation of Secretory TNT CAR T-Cells

### Construction of NFAT Enhancer-Linked IL-12 & LEC Genes

An NFAT motif found from nucleotides −286 to −258 upstream of the human IL-2 gene transcriptional activation site is utilized for inducible constructs (denoted: (NFAT)₆). The underlined sequence indicates NFAT binding site and the italicized sequence indicates an AP-1 binding site (Fiering, S. et al. (1990) Genes Dev. 4(10):1823-1834) (SEQ ID NO: 57):

₋₂₈₆GGAGGAAAAACTGTTTCATACAGAAGGCG₋₂₅₈

Succeeding these NFAT motif repeats are nucleotides −70 through +47 of the IL-2 gene promoter, which retains its TATA box and transcriptional start sites. This minimal IL-2 promoter sequence is read directly to the IL-12 or LEC coding DNA sequences (FIGS. 6A & 6B). IL-12 or LEC are, therefore, only be expressed after proper T cell activation and binding of NFAT to the minimal IL-2 promoter, allowing for transcriptional initiation.

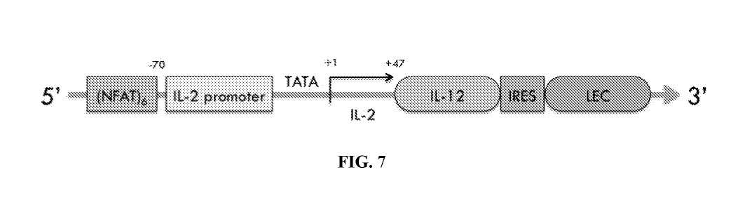

The inducible IL-12 gene is synthesized by Genewiz Gene Synthesis services. A restriction enzyme site is added at +47 to allow for subcloning of LEC or substitute genes into this inducible construct. Alternatively, for construction of a dual inducible IL-12-LEC gene, IL-12 and LEC genes are subcloned downstream from the minimal IL-2 promoter sequence and separated by an internal ribosome entry site shown below in FIG. 7.

Subcloning of TNT-CAR and inducible genes into lentiviral plasmids Separately, NovaBlue Singles™ chemically-competent E. coli cells is transformed with TNT-CAR and inducible plasmid cDNAs. Following growth of the transformed E. coli cells, the TNT-CAR and inducible plasmids are purified and digested with the appropriate restriction enzymes to be inserted into an HIV-1-based lentiviral vector containing HIV-1 5′ and 3′ long terminal repeats (LTRs), packaging signal (Ψ), EF1α promoter, internal ribosome entry site (IRES), woodchuck hepatitis virus post-transcriptional regulatory element (WPRE), and simian virus 40 origin (SV40) via overnight T₄ DNA ligase reaction (New England Biosciences; Ipswich, Mass.). NovaBlue Singles™ chemically-competent E. coli cells are then be transformed with the resulting TNT-CAR- and inducible gene-containing lentiviral plasmid.

### Production of Lentiviral Particles

Prior to transfection, HEK 293T cells are seeded at 4.0×10⁶ cells/100 mm tissue-culture-treated plate in 10 mL complete-Tet-DMEM and incubated overnight at 37° C. in a humidified 5% CO₂ incubator. Once 80-90% confluent, HEK 293T cells are co-transfected with TNT-CAR or inducible gene lentiviral vectors and lentiviral packaging vectors containing genes necessary to form lentiviral envelope & capsid components. A proprietary reaction buffer and polymer to facilitate the formation of vector-containing nanoparticles that bind HEK 293T cells are also added. After incubating the transfected-HEK 293T cell cultures for 4 hours at 37° C., the transfection medium is replaced with 10 mL fresh complete Tet DMEM. HEK 293T cells are then incubated for an additional 48 hours, after which cell supernatants are harvested and tested for lentiviral particles via sandwich ELISA against p24, the main lentiviral capsid protein. Lentivirus-containing supernatants are aliquoted and stored at −80° C. until use for transduction of target CD4⁺ and CD8⁺ T cells.

Purification, Activation, and Enrichment of Human CD4⁺ and CD8⁺ Peripheral Blood T-Cells

Peripheral blood mononuclear cells (PBMCs) enriched by density gradient centrifugation with Ficoll-Paque Plus (GE Healthcare; Little Chalfont, Buckinghamshire, UK) aree recovered and washed by centrifugation with PBS containing 0.5% bovine serum albumin (BSA) and 2 mM EDTA. MACS CD4⁺ and CD8⁺ MicroBeads (Miltenyi Biotec; San Diego, Calif.) kits are used to isolate these human T-cell subsets using magnetically activated LS columns to positive select for CD4⁺ and CD8⁺ T-cells. Magnetically-bound T-cells are then removed from the magnetic MACS separator, flushed from the LS column, and washed in fresh complete medium. The purity of CD4⁺ and CD8⁺ T-cell populations are assessed by flow cytometry using Life Technologies Acoustic Attune® Cytometer, and are enriched by Fluorescence-Activated Cell Sorting performed at USC's flow cytometry core facilities if needed. CD4⁺ and CD8⁺ T-cells mixed 1:1 are maintained at a density of 1.0×10⁶ cells/mL in complete medium supplemented with 100 IU/mL IL-2 in a suitable cell culture vessel, to which α-CD³/α-CD28 Human T-cell Dynabeads (Life Technologies; Carlsbad, Calif.) is added to activate cultured T cells. T-cells are then be incubated at 37° C. in a 5% CO₂ incubator for 2 days prior to transduction with TNT-CAR lentiviral particles.

Lentiviral Transduction of CD4⁺ CD8⁺ T-Cells

Activated T-cells are collected and dead cells are removed by Ficoll-Hypaque density gradient centrifugation or the use of MACS Dead Cell Removal Kit (Miltenyi Biotec; San Diego, Calif.). In a 6-well plate, activated T-cells are plated at a concentration of 1.0×10⁶ cells/mL in complete medium. Cells are transduced by the various methods below so that the transduction procedure that yields the greatest transduction efficiencies of viable cells are selected for successive TNT-CAR.IL-12-LEC cell production.

### Lentiviral Transduction Via Spinfection

TNT-CAR or inducible gene lentiviral particles are added to cell suspensions along with 4 μg/mL Polybrene, a cationic polymer that aids transduction by facilitating interaction between lentiviral particles and the target cell surface. Cells are centrifuged at 800×g for 1 hr at 32° C. and subsequently incubated overnight. Following centrifugation, lentivirus-containing medium is aspirated and cell pellets are resuspended in fresh complete medium with 100 IU/mL IL-2. Cells are placed in a 5% CO₂ humidified incubator at 37° C. overnight. On the following day, depleted medium is aspirated, and lentivirus supernatant is added again to cells. Three days post-transduction, cells are pelleted and resuspended in fresh complete medium with IL-2 and 400 μg/mL Geneticin (G418 sulfate) (Life Technologies; Carlsbad, Calif.).

### Lentiviral Transduction Via RetroNectin

RetroNectin is plated to non-tissue culture-treated plates via overnight incubation in PBS. Following, TNT-CAR or inducible gene lentiviral particles are added to the RetroNectin-coated plates. After a half-day incubation, lentiviral supernatants are aspirated, and CD4⁺ CD8⁺ T cells mixed 1:1 is added to the RetroNectin-lentivirus-coated plates in fresh complete medium with 100 IU/mL IL-2 and placed in a 5% CO₂ humidified incubator at 37° C. for 3 days. Afterward, cells are pelleted and resuspended in fresh complete medium with IL-2 and 400 μg/mL Geneticin.

### TNT-CAR.IL-12-LEC Cell Selection Via Antibiotic Selection

To select for TNT-CAR.IL-12-LEC cells using antibiotics, two antibiotic resistance genes are introduced separately to the TNT-CAR and inducible IL-12-LEC lentiviral vectors. In a two-step transduction procedure, CD4⁺ CD8⁺ T cells are transduced with TNT-CAR lentiviruses via spinfection or RectroNectin described above. Following one week of culture with the first antibiotic, viable cells are purified and will undergo a second transduction with inducible IL-12-LEC lentiviruses via spinfection or RectroNectin. Post-transduction, cells are incubated in the culture medium containing both antibiotics to select for TNT-CAR.IL-12-LEC-containing cells.

### TNT-CAR.IL-12-LEC Cell Selection Via FACS

Briefly, T cell cultures post-transduction are labeled with a biotin-conjugated α-idiotype TNT antibody to identify the scFv region of the TNT-CAR. Successively, a fluorescein isothiocyanate-conjugated streptavidin is used to bind α-TNT. Cells are kept on ice and transferred to USC's Flow Cytometry Core facility for enrichment of TNT-CAR cells via fluorescence activated cell sorting (FACS). Cell sorting is performed by the core's personnel using a BD FACSAria™ II (BD Biosciences; Franklin Lakes, N.J.). For enrichment of inducible-IL-12-LEC containing cells, a GFP reporter gene is used for positive selection of transduced cells.

### Example 3

### Construction of NFAT Enhancer-Linked IL-12 & LEC Genes

Eight repeats of an NFAT motif found from nucleotides −286 to −258 upstream of the human IL-2 gene transcriptional activation site are utilized for inducible constructs (denoted: (NFAT)₈). The underlined sequence indicates NFAT binding site and the italicized sequence indicates an AP-1 binding site (SEQ ID NO: 57):

₋₂₈₆GGAGGAAAAAC [symbol] TACAGAAGGCG₋₂₅₈

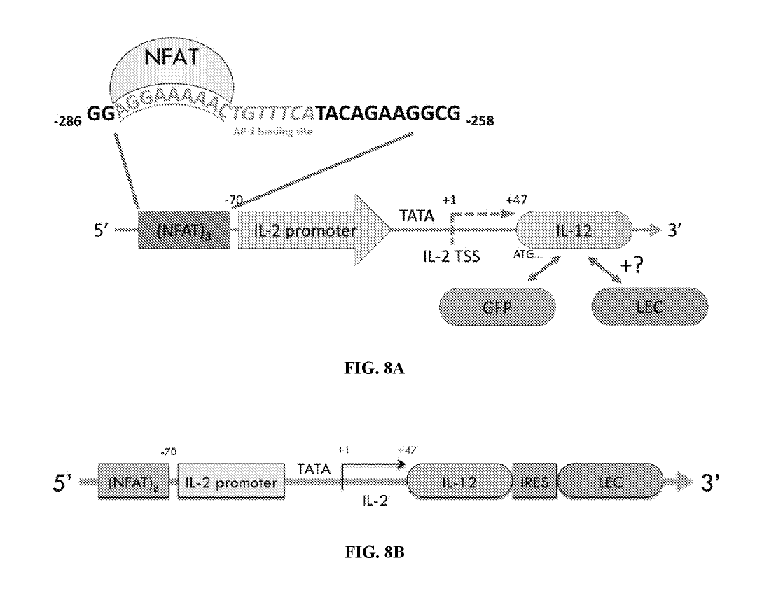

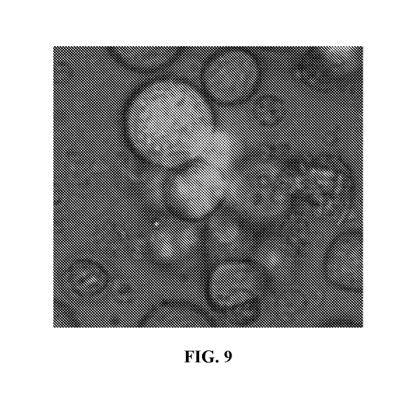

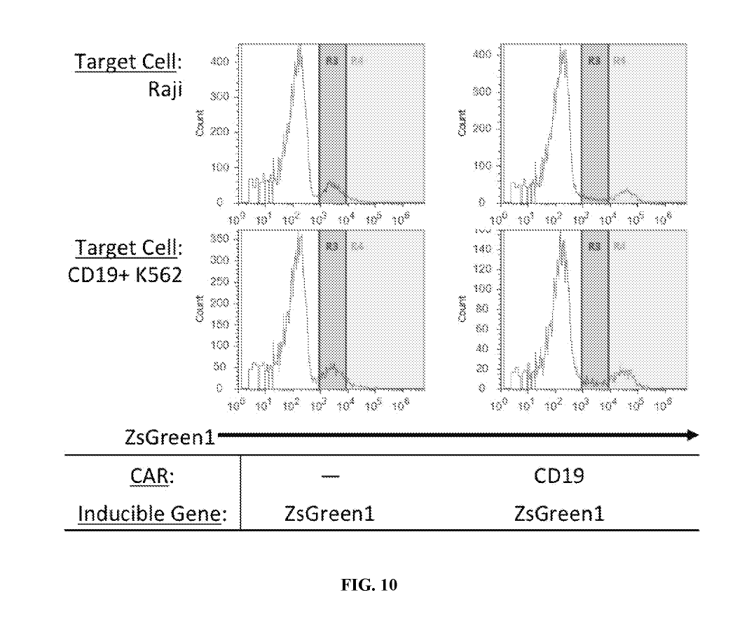

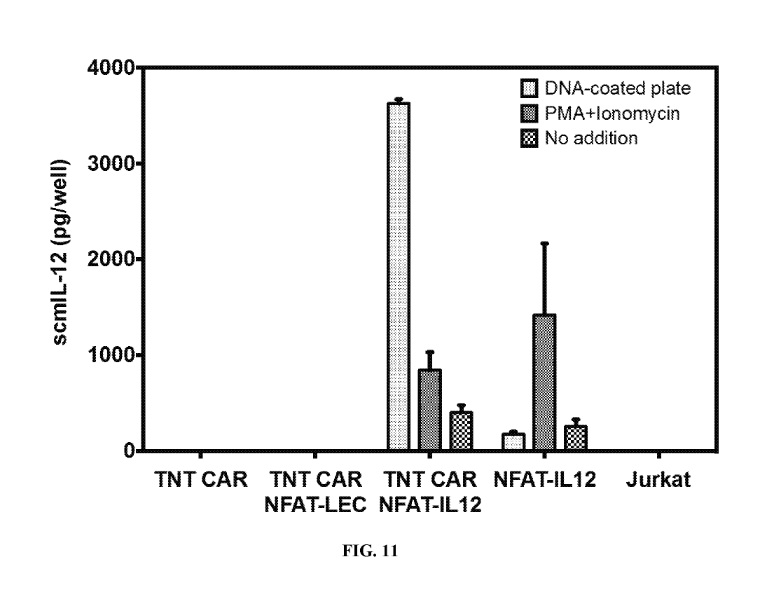

Succeeding these NFAT motif repeats are nucleotides −70 through +47 of the IL-2 gene promoter, which retains its TATA box and transcriptional start sites. This minimal IL-2 promoter sequence will read directly to the IL-12 or LEC coding DNA sequences (FIG. 8A). IL-12 or LEC will, therefore, only be expressed after proper T cell activation and binding of NFAT to the minimal IL-2 promoter, allowing for transcriptional initiation.

The inducible IL-12 gene is synthesized by Genewiz Gene Synthesis services. A restriction enzyme site is added at +47 to allow for subcloning of LEC or substitute genes into this inducible construct. Alternatively, for construction of a dual inducible IL-12-LEC gene, IL-12 and LEC genes are subcloned downstream from the minimal IL-2 promoter sequence and separated by an internal ribosome entry site shown in FIG. 8B.

Subcloning of TNT-CAR and Inducible Genes into Lentiviral Plasmids

Separately, NEB® 5-alpha chemically-competent E. coli cells are transformed with TNT-CAR and inducible plasmid cDNAs (New England Biosciences; Ipswich, Mass.). Following growth of the transformed E. coli cells, the TNT-CAR and inducible plasmids are purified and digested with the appropriate restriction enzymes to be inserted into an HIV-1-based lentiviral vector containing HIV-1 5′ and 3′ long terminal repeats (LTRs), packaging signal (Ψ), EF1α promoter, internal ribosome entry site (IRES), woodchuck hepatitis virus post-transcriptional regulatory element (WPRE), and simian virus 40 origin (SV40) via overnight T₄ DNA ligase reaction (New England Biosciences; Ipswich, Mass.). NEB® 5-alpha chemically-competent E. coli cells will then be transformed with the resulting TNT-CAR- and inducible gene-containing lentiviral plasmid.

### Production of Lentiviral Particles

Prior to transfection, 293 LTV cells (a subclone of HEK 293 T cells selected for superior lentivirus producibility) are seeded at 4.0×10⁶ cells/150 cm² tissue-culture-treated plate in 10 mL complete DMEM supplemented with 10% dialyzed FCS plus penicillin/streptomycin and incubated overnight at 37° C. in a humidified 5% CO₂ incubator. At 80-90% confluency and two hours prior to transfection, the medium is exchanged with 10 ml DMEM supplemented with 10% dialysed FCS without penicillin/streptamycin. After two hours, cells are co-transfected with TNT-CAR and/or inducible gene lentiviral plasmids plus the packaging and envelope plasmids containing lentiviral genes necessary to form lentiviral capsid & envelope components. A proprietary reaction buffer and polymer solution developed by Takara/Clontech (Mountain View, Calif.) are added to facilitate the formation of plasmid-containing nanoparticles that bind 293 LTV cells. After incubating the transfected-293 LTV cell cultures for 24 hours at 37° C., the transfection medium is replaced with 10 mL fresh complete DMEM supplemented with 10% dialyzed FCS plus penicillin/streptomycin. Following, lentivirus-containing supernatant is collected every 24 hours for 3 days. After the final collection, supernatant is centrifuged at 350×g at 4° C. and filtered of residual cells and debris. Lentivirus-containing supernatant will then be ultracentrifuged at 20,000×g for 2 hours at 4° C. and resuspeneded in PBS containing 7% trehalose and 1% BSA. Lentivirus-containing supernatants are aliquoted and stored at −80° C. until use for transduction of target effector cells.

Purification, Activation, and Enrichment of Human CD4⁺ and CD8⁺ Peripheral Blood T-Cells

Peripheral blood mononuclear cells (PBMCs) enriched by density gradient centrifugation with Ficoll-Paque Plus (GE Healthcare; Little Chalfont, Buckinghamshire, UK) are recovered and washed by centrifugation with PBS containing 0.5% bovine serum albumin (BSA) and 2 mM EDTA. T-cell enrichment kits (Stem Cell Technologies) are used to magnetically isolate PBMCs for CD4⁺ and CD8⁺ T-cells. The purity of CD4⁺ and CD8⁺ T-cell populations are assessed by flow cytometry using Life Technologies Acoustic Attune® Cytometer, and are enriched by Fluorescence-Activated Cell Sorting performed at USC's flow cytometry core facilities if needed. CD4⁺ and CD8⁺ T-cells mixed 1:1 are maintained at a density of 1.0×10⁶ cells/mL in complete 50% Click's media/50% RPMI-1640 supplemented with 100 IU/mL IL-2 in a suitable cell culture vessel, to which α-CD3/α-CD28 Human T-cell activator beads (Stem Cell Technologies) are added to activate cultured T cells. T-cells will then be incubated at 37° C. in a 5% CO₂ incubator for 2 days prior to transduction with TNT-CAR lentiviral particles.

Lentiviral Transduction of CD4⁺ CD8⁺ T-Cells

Activated T-cells are collected and dead cells are removed by Ficoll-Hypaque density gradient centrifugation or the use of MACS Dead Cell Removal Kit (Miltenyi Biotec; San Diego, Calif.). In a 6-well plate, activated T-cells are plated at a concentration of 1.0×10⁶ cells/mL in complete medium. Cells are transduced with the lentiviral particles using Lentiblast transfection aid (Oz Biosciences, San Diego, Calif.). To increase transduction efficiency of difficult-to-transduce cells, namely NK-92MI, cells may be centrifuged at 800×g for 1 hr at 32° C. in a RetroNectin-coated plate (Takara/Clontech; Mountain View, Calif.) for 1 hr at 32° C. before overnight incubation. On the morning following transduction, lentivirus-containing depleted medium is exchanged with fresh complete RPMI. The transduced cells with be cultured until further downstream assay and analysis.

### TNT-CAR.IL-12-LEC Cell Selection Via Antibiotic Selection

To select for TNT-CAR.IL-12-LEC cells using antibiotics, two antibiotic resistance genes are introduced separately to the TNT-CAR and inducible IL-12-LEC lentiviral plasmids. In a two-step transduction procedure, CD4⁺ CD8⁺ T cells are transduced with TNT-CAR lentiviruses via spinfection or RectroNectin described above. Following one week of culture with the first antibiotic, viable cells are purified and will undergo a second transduction with inducible IL-12-LEC lentiviruses via spinfection or RectroNectin. Post-transduction, cells are incubated in the culture medium containing both antibiotics to select for TNT-CAR.IL-12-LEC-containing cells.

### TNT-CAR.IL-12-LEC Cell Selection Via FACS

Briefly, T cell cultures post-transduction are labeled with a biotin-conjugated α-idiotype TNT antibody to identify the scFv region of the TNT-CAR. Successively, a fluorescein isothiocyanate-conjugated streptavidin is used to bind α-TNT. Cells are kept on ice and transferred to USC's Flow Cytometry Core facility for enrichment of TNT-CAR cells via fluorescence activated cell sorting (FACS). Cell sorting is performed by the core's personnel using a BD FACSAria™ II (BD Biosciences; Franklin Lakes, N.J.). For enrichment of inducible-IL-12-LEC containing cells, a green fluorescent protein (GFP) reporter gene is used for positive selection of transduced cells.

### Assays for Induction of Inducible Genes

### Stimulation of CAR NFAT Cells

CAR NFAT cells are plated at densities ranging from 0.5-5.0×10⁵ cells/mL in complete medium. To induce NFAT genes, one of the following conditions are administered: target cells or target antigen are added at a 1:1 CAR-NFAT-to-target cell ratio; cells are stimulated with 1-2% PHA, or PMA (final concentration: 10 ng/mL) plus ionomycin (500 ng/mL); or cells are given diluent control. After an incubation overnight, CAR-NFAT cells or supernatants are harvested for downstream analysis.

### Flow Cytometry

Harvested cells are washed in PBS twice by centrifugation. Following, cells will resuspended in PBS 2% FCS and undergo FcR blocking with subsequent staining with fluorescent antibodies against the CAR cells (i.e. anti-scFv, anti-CD3) and target cell antigen (e.g. anti-CD19). After a one-hour incubation at 4° C., cells are washed in PBS 2% FCS and analyzed for induced gene expression using Life Technologies Attune® Acoustic Focusing Cytometer.

### ELISA for LEC and IL-12

Stimulated cells are centrifuged and supernatants transferred to a 96-well plate coated with the proper capture antibody (see table below). After an overnight incubation at 4° C., the ELISA plate is washed in PBS 0.05% Tween-20 and then probed with the appropriate biotinylated detection antibody conjugated. Following, the plate is incubated with streptavidin-HRP, developed using TMB substrate (Biolegend; San Diego, Calif.). The colorigenic TMB reaction is stopped with 1 M H₂SO₄. Absorbances at 450 nm are measured on BioTek's Synergy HT microplate reader (BioTek; Winooski, Vt.).

TABLE 3

| | LEC | IL-12 |
|---|---|---|
| Capture | Mouse anti-human LEC | Rat anti-mouse IL-12 p35 subunit |
| | Clone: 1.2_5G3-1D7 | Clone: C18.2 (eBioscience; San |
| | (Peprotech; Rocky Hill, | Diego, CA) |
| | NJ) | |
| Detection | Goat anti-human LEC | Rat anti-mouse IL-12 p40 subunit |
| | Clone: polyclonal | Clone: C17.8 (eBioscience; San |
| | (Peprotech; Rocky Hill, | Diego, CA) |
| | NJ) | |

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### SEQUENCE LISTING

TNT-1 CDHR1, SEQ ID NO: 1:
GFSLTDYG
TNT-1 CDHR2, SEQ ID NO: 2:
IWGGGST
TNT-1 CDHR3, SEQ ID NO: 3:
AKEKRRGYYYAMDY
TNT-1 CDLR1, SEQ ID NO: 4:
SSVSSSY
TNT-1 CDLR2, SEQ ID NO: 5:
STS
TNT-1 CDLR3, SEQ ID NO: 6:
QQYSGYPLT
TNT-1 Heavy Chain Variable Region Sequence,
SEQ ID NO: 7:
CAGGTGCAGCTGAAGGAGTCAGGACCTGGCCTGGTGGCGCCCTCACAGA
GCCTGTCCATCACATGCACTGTCTCAGGGTTCTCATTAACCGACTATGG
TGTAAGGTGGATTCGCCAGCCTCCAGGAAAGGGTCTGGAGTGGCTGGGA
GTAATATGGGGTGGTGGAAGCACATACTATAATTCAGCTCTCAAATCCA
GACTGAGCATCAGCAAGGACAACTCCAAGAGCCAAGTTTTCTTAAAAAT
GAACAGTCTGCAAACTGATGACACAGCCATGTACTACTGTGCCAAAGAG
AAACGGAGGGGGTATTACTATGCTATGGACTACTGGGGTCAAGGAACCT
CAGTCACCGTCTCCTCA
TNT-1 Light Chain Variable Region Sequence,
SEQ ID NO: 8:
GGAGAAAATGTGCTCACCCAGTCTCCAGCAATCATGTCTGCATCTCCAG
GGGAAAAGGTCACCATGACCTGCAGGGCCAGCTCAAGTGTAAGTTCCAG
TTACTTGCACTGGTACCAGCAGAAGTCAGGTGCCTCCCCCAAACTCTGG
ATTTATAGCACATCCAACTTGGCTTCTGGAGTCCCTGCTCGCTTCAGTG
GCAGTGGGTCTGGGACCTCTTACTCTCTCACAATCAGCAGTGTGGAGGC
TGAAGATGCTGCCACTTATTACTGCCAGCAGTACAGTGGTTACCCACTC
ACGTTCGGAGGGGGGACCAAGCTGGAAATAAAA
TNT-2 CDHR1, SEQ ID NO: 9:
GYSFTGYY
TNT-2 CDHR2, SEQ ID NO: 10:
INPYNGAT
TNT-2 CDHR3, SEQ ID NO: 11:
ARLDRGDY
TNT-2 CDLR1, SEQ ID NO: 12:
ENVVTY
TNT-2 CDLR2, SEQ ID NO: 13:
GAS
TNT-2 CDLR3, SEQ ID NO: 14:
GQGYSYPYT
TNT-2 Heavy Chain Variable Region Sequence,
SEQ ID NO: 15:
GAGGTACAGCTGCAGCAGTCTGGACCTGAGCTGGTGAAGCCTGGGGCTT
CAGTGAAGATATCCTGCAAGGCTTCTGGTTACTCATTCACTGGCTACTA
CATGCACTGGGTGAAGCAAAGCCATGTAAAGAGCCTTGAGTGGATTGGA
CGTATTAATCCTTACAATGGTGCTACTAGCTACAACCAGAATTTCAAGG
ACAAGGCCAGCTTGACTGTAGATAAGTCCTCCAGCACAGCCTACATGGA
GCTCCACAGCCTGACATCTGAGGACTCTGCAGTCTATTACTGTGCAAGA
CTAGACCGGGGGGACTACTGGGGTCAAGGAACCTCAGTCACCGTCTCCT
CA
TNT-2 Light Chain Variable Region Sequence,
SEQ ID NO: 16:
AACATTGTAATGACCCAATCTCCCAAATCCATGTCCATGTCAGTAGGAG
AGAGGGTCACCTTGACCTGCAAGGCCAGTGAGAATGTGGTTACTTATGT
TTCCTGGTATCAACAGAAACCAGAGCAGTCTCCTAAACTGCTGATATAC
GGGGCATCCAACCGGTACACTGGGGTCCCCGATCGCTTCACAGGCAGTG
GATCTGCAACAGATTTCACTCTGACCATCAGCAGTGTGCAGGCTGAAGA
CCTTGCAGATTATCACTGTGGACAGGGTTACAGCTATCCGTACACGTTC
GGAGGGGGGACAAAGTTGGAAATAAAACGTACG
TNT-3 CDHR1, SEQ ID NO: 17:
GYTFTRYW
TNT-3 CDHR2, SEQ ID NO: 18:
IYPGNSDT
TNT-3 CDHR3, SEQ ID NO: 19:
ARGEEIGVRRWFAY
TNT-3 CDLR1, SEQ ID NO: 20:
QSISNY
TNT-3 CDLR2, SEQ ID NO: 21:
YAS
TNT-3 CDLR3, SEQ ID NO: 22:
QQSNSWPLT
TNT-3 Heavy Chain Variable Region Sequence,
SEQ ID NO: 23:
CAGGTCCAACTGCAGCAGTCAGGAGCTGAACTGGTCAAGACTGGGGCCT
CAGTGAAGATGTCCTGCAAGGCTTCTGGCTACACCTTTACCAGATACTG
GATGCACTGGGTAAAACAGAGGCCTGGACAGGCTCTGGAATGGATTGGC
GCTATTTATCCTGGAAATAGTGATACTAGCTACTACCAGAAGTTCAAGG
GCAAGGCCAAACTGACTGCAGTCACATCTGCCAGCACTGCCTACATGGA
GCTCAGCAGCCTGACATCTGAGGACTCTGCCGTCTATTACTGTGCAAGA
GGGGAGGAAATAGGGGTACGACGCTGGTTTGCTTACTGGGGCCAAGGGA
CTCTGGTCACTGTCTCTGCA
TNT-3 Light Chain Variable Region Sequence,
SEQ ID NO: 24:
GATATTGTGC TAACTCAGTC TCCAGCCACC CTGTCTGTGA
CTCCAGGAGA TAGAGTCAGT CTTTCCTGCA GGGCCAGGCA
AAGTATTAGC AACTACCTAC ACTGGTATCA ACAAAAATCA
CATGAGTCTC CAAGGCTTCT CATCAAGTAT GCTTCCCAGT
CCATCTCTGG CATCCCCTCC AGGTTCAGTG GCAGTGGATC
AGGGACAGAT TTCACTCTCA GTATCAACAG TGTGGAGACT
GAAGATTTTG GAATGTATTT CTGTCAACAG AGTAACAGCT
GGCCGCTCAC GTTCGGTGCT GGGACCAAGC TGGAAATAAA
A
NHS76 CDHR1, SEQ ID NO: 25:
SGYYWG
NHS76 CDHR2, SEQ ID NO: 26:
SIYHSGSTYYNPSLKS
NHS76 CDHR3, SEQ ID NO: 27:
GKWSKFDY
NHS76 CDLR1, SEQ ID NO: 28:
QGDSLRSYYAS
NHS76 CDLR2, SEQ ID NO: 29:
GKNNRPS
NHS76 CDLR3, SEQ ID NO: 30:
NSRDSSGNHVV
NHS76 Heavy Chain Variable Region Sequence,
SEQ ID NO: 31:
CAGGTGCAGC TGCAGGAGTC CGGCCCAGGA CTGGTGAAGC
CTTCGGAGAC CCTGTCCCTC ACCTGCGCTG TCTCTGGTTA
CTCCATCAGC AGTGGTTACT ACTGGGGCTG GATTCGGCAG
CCCCCAGGGA AGGGGCTGGA GTGGATTGGG AGTATCTATC
ATAGTGGGAG CACCTACTAC AACCCGTCCC TCAAGAGTCG
AGTCACCATA TCAGTAGACA CGTCCAAGAA CCAGTTCTCC
CTGAAGCTGA GCTCTGTGAC CGCCGCAGAC ACGGCCGTGT
ATTACTGTGC AAGAGGGAAG TGGTCGAAGT TTGACTATTG
GGGCCAAGGC ACCCTGGTCA CCGTCTCTTC A
NHS76 Light Chain Variable Region Sequence,
SEQ ID NO: 32:
TCCTCTGAGC TGACTCAGGA CCCTGCTGTG TCTGTGGCCT
TGGGACAGAC AGTCAGGATC ACATGCCAAG GAGACAGCCT
CAGAAGCTAT TATGCAAGCT GGTACCAGCA GAAGCCAGGA
CAGGCCCCTG TACTTGTCAT CTATGGTAAA AACAACCGGC
CCTCAGGGAT TCCAGACCGA TTCTCTGGCT CCAGCTCAGG
AAACACAGCT TCCTTGACCA TCACTGGGGC TCAGGCGGAA
GATGAGGCTG ACTATTACTG TAACTCCCGG GACAGCAGTG
GTAACCATGT GGTATTCGGC GGAGGGACCA AGCTGACCGT
CCTA
Human CD8 alpha hinge domain, SEQ ID NO: 33:
PAKPTTTPAPRPPTPAPTIASQPLSLRPEACRPAAGGAVHTRGLDFACD
IY
Mouse CD8 alpha hinge domain, SEQ ID NO: 34:
KVNSTTTKPVLRTPSPVHPTGTSQPQRPEDCRPRGSVKGTGLDFACDIY
Cat CD8 alpha hinge domain, SEQ ID NO: 35:
PVKPTTTPAPRPPTQAPITTSQRVSLRPGTCQPSAGSTVEASGLDLSCD
IY
Human CD8 alpha transmembrane domain,
SEQ ID NO: 36:
IYIWAPLAGTCGVLLLSLVIT
Mouse CD8 alpha transmembrane domain,
SEQ ID NO: 37: IWAPLAGICVALLLSLIITLI
Rat CD8 alpha transmembrane domain,
SEQ ID NO: 38: IWAPLAGICAVLLLSLVITLI
The 4-1BB costimulatory signaling region,
SEQ ID NO: 39:
KRGRKKLLYIFKQPFMRPVQTTQEEDGCSCRFPEEEEGGCEL
CD28 Sequence (SEQ ID NO: 40):
MLRLLLALNL FPSIQVTGNK ILVKQSPMLV AYDNAVNLSC
KYSYNLFSRE FRASLHKGLDSAVEVCVVYG NYSQQLQVYS
KTGFNCDGKL GNESVTFYLQ NLYVNQTDIY
FCKIEVMYPPPYLDNEKSNG TIIHVKGKHL CPSPLFPGPS
KPFWVLVVVG GVLACYSLLVTVAFIIFWVR SKRSRLLHSD
YMNMTPRRPG PTRKHYQPYA PPRDFAAYRS
CD3 zeta signaling domain (SEQ ID NO: 41):
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR
“B7.1” NP_005182.1,
SEQ ID NO: 42:
MGHTRRQGTS PSKCPYLNFF QLLVLAGLSH FCSGVIHVTK
EVKEVATLSC GHNVSVEELA QTRIYWQKEK KMVLTMMSGD
MNIWPEYKNR TIFDITNNLS IVILALRPSD EGTYECVVLK
YEKDAFKREH LAEVTLSVKA DFPTPSISDF EIPTSNIRRI
ICSTSGGFPE PHLSWLENGE ELNAINTTVS QDPETELYAV
SSKLDFNMTT NHSFMCLIKY GHLRVNQTFN WNTTKQEHFP
DNLLPSWAIT LISVNGIFVI CCLTYCFAPR CRERRRNERL
RRESVRPV
“CCL19” NP_006265.1,
SEQ ID NO: 43:
MALLLALSLL VLWTSPAPTL SGTNDAEDCC LSVTQKPIPG
YIVRNFHYLL IKDGCRVPAV VFTTLRGRQL CAPPDQPWVE
RIIQRLQRTS AKMKRRSS
“CCL20”

### An Examples Include NP_004582.1, SEQ ID NO: 44:

MCCTKSLLLA ALMSVLLLHL CGESEAASNF DCCLGYTDRI
LHPKFIVGFT RQLANEGCDI NAIIFHTKKK LSVCANPKQT
WVKYIVRLLS KKVKNM
and NP_001123518.1, SEQ ID NO: 45:
MCCTKSLLLA ALMSVLLLHL CGESEASNFD CCLGYTDRIL
HPKFIVGFTR QLANEGCDIN AIIFHTKKKL SVCANPKQTW
VKYIVRLLSK KVKNM
“CD40L” NP_000065.1, SEQ ID NO: 46:
MIETYNQTSP RSAATGLPIS MKIFMYLLTV FLITQMIGSA
LFAVYLHRRL DKIEDERNLH EDFVFMKTIQ RCNTGERSLS
LLNCEEIKSQ FEGFVKDIML NKEETKKENS FEMQKGDQNP
QIAAHVISEA SSKTTSVLQW AEKGYYTMSN NLVTLENGKQ
LTVKRQGLYY IYAQVTFCSN REASSQAPFI ASLCLKSPGR
FERILLRAAN THSSAKPCGQ QSIHLGGVFE LQPGASVFVN
VTDPSQVSHG TGFTSFGLLK L
“CD137L” NP_003802.1, SEQ ID NO: 47:
MEYASDASLD PEAPWPPAPR ARACRVLPWA LVAGLLLLLL
LAAACAVFLA CPWAVSGARA SPGSAASPRL REGPELSPDD
PAGLLDLRQG MFAQLVAQNV LLIDGPLSWY SDPGLAGVSL
TGGLSYKEDT KELVVAKAGV YYVFFQLELR RVVAGEGSGS
VSLALHLQPL RSAAGAAALA LTVDLPPASS EARNSAFGFQ
GRLLHLSAGQ RLGVHLHTEA RARHAWQLTQ GATVLGLFRV
TPEIPAGLPS PRSE
“GITRL” NP_005083.2, SEQ ID NO: 48:
MTLHPSPITC EFLFSTALIS PKMCLSHLEN MPLSHSRTQG
AQRSSWKLWL FCSIVMLLFL CSFSWLIFIF LQLETAKEPC
MAKFGPLPSK WQMASSEPPC VNKVSDWKLE ILQNGLYLIY
GQVAPNANYN DVAPFEVRLY KNKDMIQTLT NKSKIQNVGG
TYELHVGDTI DLIFNSEHQV LKNNTYWGII LLANPQFIS
“GM-CSF” UniProt Reference No.
P04141 - CSF2_HUMAN (SEQ ID NO: 49):
MWLQSLLLLG TVACSISAPA RSPSPSTQPW EHVNAIQEAR
RLLNLSRDTA AEMNETVEVI SEMFDLQEPT CLQTRLELYK
QGLRGSLTKL KGPLTMMASH YKQHCPPTPE TSCATQIITF
ESFKENLKDF LLVIPFDCWE PVQE
“IL-12” SEQ ID NO: 50:
CATGGCCATG TGTCATCAGC AGCTGGTCAT CAGCTGGTTC
AGCCTGGTGT TCCTGGCCAG CCCCCTGGTG GCCATCTGGG
AGCTGAAGAA AGACGTGTAC GTGGTGGAGC TGGACTGGTA
TCCCGACGCC CCTGGCGAGA TGGTGGTGCT GACCTGCGAC
ACCCCCGAAG AGGACGGCAT CACCTGGACC CTGGACCAGA
GCAGCGAGGT GCTGGGCAGC GGCAAGACCC TGACCATCCA
GGTCAAAGAG TTCGGCGACG CCGGCCAGTA CACCTGCCAC
AAGGGCGGCG AAGTGCTGTC CCACAGCCTG CTGCTGCTGC
ACAAGAAAGA GGATGGCATC TGGTCCACCG ACATCCTGAA
GGACCAGAAA GAGCCCAAGA ACAAGACCTT CCTGCGGTGC
GAGGCCAAGA ACTACAGCGG CCGGTTCACC TGTTGGTGGC
TGACCACCAT CAGCACCGAC CTGACCTTCA GCGTGAAGAG
CAGCCGGGGC AGCAGCGACC CTCAGGGCGT GACCTGCGGA
GCCGCCACCC TGAGCGCCGA GAGAGTGCGG GGCGACAACA
AAGAGTACGA GTACAGCGTC GAGTGCCAGG AAGATAGCGC
CTGCCCTGCC GCCGAGGAAA GCCTGCCCAT CGAGGTGATG
GTGGACGCCG TGCACAAGCT GAAGTACGAG AACTACACCT
CCAGCTTTTT CATCCGGGAC ATCATCAAGC CCGACCCCCC
CAAGAACCTG CAGCTGAAGC CCCTGAAGAA CAGCCGGCAG
GTGGAGGTGT CCTGGGAGTA CCCTGACACC TGGTCCACCC
CCCACAGCTA CTTCAGCCTG ACCTTCTGTG TGCAGGTGCA
GGGCAAGAGC AAGCGGGAGA AGAAAGACCG GGTGTTCACC
GACAAGACCA GCGCCACCGT GATCTGCCGG AAGAACGCCA
GCATCAGCGT GCGGGCCCAG GACCGGTACT ACAGCAGCTC
CTGGTCCGAG TGGGCCAGCG TGCCCTGCAG CGGCGGAGGG
GGCGGAGGAA GCCGGAACCT GCCCGTGGCT ACCCCCGACC
CCGGCATGTT CCCCTGCCTG CACCACAGCC AGAACCTGCT
GCGGGCCGTG AGCAACATGC TGCAGAAGGC CCGGCAGACC
CTGGAATTCT ACCCCTGCAC CAGCGAGGAA ATCGACCACG
AGGACATCAC CAAGGATAAG ACCAGCACCG TGGAGGCCTG
CCTGCCCCTG GAACTGACCA AGAACGAGAG CTGTCTGAAC
TCTCGGGAGA CAAGCTTCAT CACCAACGGC TCTTGCCTGG
CCAGCAGAAA GACCAGCTTC ATGATGGCCC TGTGCCTGAG
CAGCATCTAC GAGGACCTGA AGATGTACCA GGTGGAGTTC
AAGACCATGA ACGCCAAGCT GCTGATGGAC CCCAAGCGGC
AGATCTTCCT GGATCAGAAC ATGCTGGCCG TGATCGACGA
GCTGATGCAG GCCCTGAACT TCAACAGCGA GACAGTGCCC
CAGAAGTCCA GCCTGGAAGA GCCCGACTTC TACAAGACCA
AGATCAAGCT GTGCATCCTC CTGCATGCCT TCCGGATCCG
GGCCGTGACC ATCGACCGGG TGATGAGCTA CCTGAACGCC
AGCTGATGAG C
“IL-2” SEQ ID NO: 51:
APTSSSTKKT QLQLEHLLLD LQMILNGINN YKNPKLTRML
TFKFYMPKKA TELKHLQCLE EELKPLEEVL NLAQSKNFHL
RPRDLISNIN VIVLELKGSE TTFMCEYADE TATIVEFLNR
WITFCQSIIS TLT
“IL-15” SEQ ID NO: 52.
WVNVISDLKKIEDLIQSMHIDATLYTESDVHPSCKVTAMKCFLLELQVI
SLESGDASIHDTVENLIILANNSLSSNGNVTESGCKECEELEKKNIKEF
LQSFVHIVQMFINTS
“IL-18” SEQ ID NO: 53.
MAAEPVEDNCINFVAMKFIDNTLYFIAEDDENLESDYFGKLESKLSVIR
NLNDQVLFIDQGNRPLFEDMTDSDCRDNAPRTIFIISMYKDSQPRGMAV
TISVKCEKISTLSCENKIISFK
EMNPPDNIKDTKSDIIFFQRSVPGHDNKMQFESSSYEGYFLACEKERDL
FKLILKKEDEL GDRSIMFTVQNED
“IL-21” SEQ ID NO: 54.
QGQDRHMIRMRQLIDIVDQLKNYVNDLVPEFLPAPEDVETNCEWSAFSC
FQKAQLKSANTGNNERIINVSIKKLKRKPPSTNAGRRQKHRLTCPSCDS
YEKKPPKEFLERFKSLLQKMIHQHLSSRTHGSEDS
“LEC” NP_004581.1,
SEQ ID NO: 55:
MKVSEAALSL LVLILIITSA SRSQPKVPEW VNTPSTCCLK
YYEKVLPRRL VVGYRKALNC HLPAIIFVTK RNREVCTNPN
DDWVQEYIKD PNLPLLPTRN LSTVKIITAK NGQPQLLNSQ
“OX40L” NP_003317.1,
SEQ ID NO: 56:
MERVQPLEEN VGNAARPRFE RNKLLLVASV IQGLGLLLCF
TYICLHFSAL QVSHRYPRIQ SIKVQFTEYK KEKGFILTSQ
KEDEIMKVQN NSVIINCDGF YLISLKGYFS QEVNISLHYQ
KDEEPLFQLK KVRSVNSLMV ASLTYKDKVY LNVTTDNTSL
DDFHVNGGEL ILIHQNPGEF CVL

Example Polynucleotide that Interacts with NFAT (SEQ ID NO: 57):

GGAGGAAAAACTGTTTCATACAGAAGGCG

Example Polynucleotide that Interacts with NFAT (SEQ ID NO: 58):

AGGAAAAAC
Hinge domain: IgG1 heavy chain hinge sequence,
SEQ ID NO: 59:
CTCGAGCCCAAATCTTGTGACAAAACTCACACATGCCCACCGTGCCCG
Transmembrane domain: CD28 transmembrane region
SEQ ID NO: 60:
TTTTGGGTGCTGGTGGTGGTTGGTGGAGTCCTGGCTTGCTATAGCTTGC
TAGTAACAGTGGCCTTTATTATTTTCTGGGTG
Intracellular domain: 4-1BB co-stimulatory
signaling region, SEQ ID NO: 61:
AAACGGGGCAGAAAGAAACTCCTGTATATATTCAAACAACCATTTATGA
GACCAGTACAAACTACTCAAGAGGAAGATGGCTGTAGCTGCCGATTTCC
AGAAGAAGAAGAAGGAGGATGTGAACTG
Intracellular domain: CD28 co-stimulatory
signaling region, SEQ ID NO: 62:
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCC
Intracellular domain: CD3 zeta signaling
region, SEQ ID NO: 63:
AGAGTGAAGTTCAGCAGGAGCGCAGACGCCCCCGCGTACCAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGCTAA
ICOS costimulatory signaling region,
SEQ ID NO: 64:
ACAAAAAAGA AGTATTCATC CAGTGTGCAC GACCCTAACG
GTGAATACAT GTTCATGAGA GCAGTGAACA CAGCCAAAAA
ATCCAGACTC ACAGATGTGA CCCTA
OX40 costimulatory signaling region,
SEQ ID NO: 65:
AGGGACCAG AGGCTGCCCC CCGATGCCCA CAAGCCCCCT
GGGGGAGGCA GTTTCCGGAC CCCCATCCAA GAGGAGCAGG
CCGACGCCCA CTCCACCCTG GCCAAGATC

1. A chimeric antigen receptor (CAR) comprising: (a) an antigen binding domain of a TNT antibody; (b) a hinge domain; (c) a transmembrane domain; (d) and an intracellular domain.

2. The chimeric antigen receptor (CAR) of claim 1, comprising: (a) an antigen binding domain of a TNT antibody; (b) a CD8 α hinge domain; (c) a CD8 α transmembrane domain; (d) a CD28 costimulatory signaling region and/or a 4-1BB costimulatory signaling region; and (e) a CD3 zeta signaling domain.

3. The CAR of claim 1, wherein the antigen binding domain of the TNT antibody comprises a TNT heavy chain variable region and a TNT light chain variable region.

4. The CAR of claim 3, further comprising a linker polypeptide located between the TNT heavy chain variable region and the TNT light chain variable region.

5. The CAR of claim 3, wherein the TNT heavy chain variable region comprises a CDR region comprising any one of SEQ ID NOs: 1-3, 9-11, 17-19, 25-27, or an equivalent of each thereof.

6. The CAR of claim 3, wherein the TNT heavy chain variable region comprises an amino acid sequence encoded by any one of SEQ ID NOs: 7, 15, 23, 31, or an equivalent of each thereof.

7. The CAR of claim 3, wherein the TNT light chain variable region a CDR region comprising any one of SEQ ID NOs: 4-6, 12-14, 28-30 or an equivalent of each thereof.

8. The CAR of claim 3, wherein the TNT light chain variable region a CDR region comprises an amino acid sequence encoded by any one of SEQ ID NOs: 8, 16, 24, 32 or an equivalent of each thereof.

9. The CAR of claim 4, wherein the linker polypeptide comprises polypeptide comprising the sequence (glycine-serine)n wherein n is an integer from 1 to 6 (SEQ ID NO: 66).

10. The CAR of claim 1, further comprising a detectable marker or a purification marker attached to the CAR.

11. The CAR of claim 5, wherein an equivalent comprises a polypeptide having at least 80% amino acid identity to the CAR or a polypeptide that is encoded by a polynucleotide that hybridizes under conditions of high stringency to the complement of a polynucleotide encoding the CAR, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

12. An isolated nucleic acid sequence encoding the CAR of claim 1 or its complement, or an equivalent of each thereof, wherein an equivalent has at least 80% sequence identity to the polynucleotide or its complement, or a polynucleotide that hybridizes under conditions of high stringency to the polynucleotide or the complement of the isolated nucleic acid encoding the CAR, wherein conditions of high stringency comprises incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water.

13. The isolated nucleic acid of claim 12, further comprising a Kozak consensus sequence located upstream of a polynucleotide encoding the antigen binding domain of the TNT antibody.

14. The isolated nucleic sequence of claim 12, wherein the isolated nucleic acid further comprises an antibiotic resistance polynucleotide.

15. A vector comprising the isolated nucleic acid sequence of any one of claim 12.

16. The vector of claim 15, wherein the vector is a plasmid.

17. The vector of claim 15, wherein the vector is a viral vector selected from the group of a retroviral vector, a lentiviral vector, an adenoviral vector, and an adeno-associated viral vector.

18. An isolated cell comprising the CAR of claim 1.

19. The isolated cell of claim 18, further comprising an isolated nucleic acid comprising an NFAT regulatory polynucleotide operatively linked to a polynucleotide encoding an immunoregulatory molecule.

20. The isolated cell claim 19, further comprising a polynucleotide encoding an antibiotic resistance polypeptide coupled to the isolated nucleic acid.

21. The isolated cell of claim 19, wherein immunoregulatory molecule is one or more selected from the group consisting of B7.1, CCL19, CCL21, CD40L, CD137L, GITRL, GM-CSF, IL-12, IL-2, low-toxicity IL-2, IL-15, IL-18, IL-21, LEC, and OX40L.

22. The isolated cell of claim 19, wherein the immunoregulatory molecule comprises IL-12 and/or GM-CSF.

23. The isolated cell of claim 19, wherein the immunoregulatory molecule comprises IL-12 and/or one or more of IL-2 and low-toxicity IL-2.

24. The isolated cell of claim 19, wherein the immunoregulatory molecule comprises IL-12 and/or IL-15.

25. The isolated cell of claim 19, wherein the immunoregulatory molecule comprises IL-12 and/or IL-21.

26. The isolated cell of claim 19, wherein the immunoregulatory molecule comprises IL-12 and/or B7.1.

27. The isolated cell of claim 19, wherein the immunoregulatory molecule comprises IL-12 and/or OX40L.

28. The isolated cell of claim 19, wherein the immunoregulatory molecule comprises IL-12 and/or CD40L.

29. The isolated cell of claim 19, wherein the immunoregulatory molecule comprises IL-12 and/or GITRL.

30. The isolated cell of claim 19, wherein the immunoregulatory molecule comprises IL-12 and/or IL-18.

31. The isolated cell of claim 19, wherein the immunoregulatory molecule comprises one or more of IL-2 and low-toxicity IL-2 and one or more of CCL19, CCL21, and LEC.

32. The isolated cell of claim 19, wherein the immunoregulatory molecule comprises IL-15 and one or more of CCL19, CCL21, and LEC.

33. The isolated cell of claim 19, wherein the immunoregulatory molecule comprises IL-21 and one or more of CCL19, CCL21, and LEC.

34. The isolated cell of claim 19, wherein the immunoregulatory molecule comprises GM-CSF and one or more of CCL19, CCL21, and LEC.

35. The isolated cell of claim 19, wherein the immunoregulatory molecule comprises OX40L and one or more of CCL19, CCL21, and LEC.

36. The isolated cell of claim 19, wherein the immunoregulatory molecule comprises CD137L and one or more of CCL19, CCL21, and LEC.

37. The isolated cell of claim 19, wherein the immunoregulatory molecule comprises B7.1 and one or more of CCL19, CCL21, and LEC.

38. The isolated cell of claim 19, wherein the immunoregulatory molecule comprises CD40L and one or more of CCL19, CCL21, and LEC.

39. The isolated cell of claim 19, wherein the immunoregulatory molecule comprises GITRL and one or more of CCL19, CCL21, and LEC.

40. The isolated cell of claim 18, wherein the cell is a prokaryotic cell or a eukaryotic cell.

41. The isolated cell of claim 18, wherein the cell is a eukaryotic cell.

42. The isolated of claim 41, wherein the eukaryotic cell is selected from an animal cell, a mammalian cell, a bovine cell, a feline cell, a canine cell, a murine cell, an equine cell or a human cell.

43. The isolated cell of claim 42, wherein the eukaryotic cell is a T-cell, B cell, or a NK cell.

44. A composition comprising the CAR of claim 1.

45. The composition of claim 44, further comprising an immunoregulatory molecule.

46. The composition of claim 45, wherein immunoregulatory molecule is one or more selected from the group consisting of B7.1, CCL19, CCL21, CD40L, CD137L, GITRL, GM-CSF, IL-12, IL-2, low-toxicity IL-2, IL-15, IL-18, IL-21, LEC, and OX40L.

47. The composition of claim 45, wherein the immunoregulatory molecule comprises IL-12 and/or GM-CSF.

48. The composition of claim 45, wherein the immunoregulatory molecule comprises IL-12 and/or one or more of IL-2 and low-toxicity IL-2.

49. The composition of claim 45, wherein the immunoregulatory molecule comprises IL-12 and/or IL-15.

50. The composition of claim 45, wherein the immunoregulatory molecule comprises IL-12 and/or IL-21.

51. The composition of claim 45, wherein the immunoregulatory molecule comprises IL-12 and/or B7.1.

52. The composition of claim 45, wherein the immunoregulatory molecule comprises IL-12 and/or OX40L.

53. The composition of claim 45, wherein the immunoregulatory molecule comprises IL-12 and/or CD40L.

54. The composition of claim 45, wherein the immunoregulatory molecule comprises IL-12 and/or GITRL.

55. The composition of claim 45, wherein the immunoregulatory molecule comprises IL-12 and/or IL-18.

56. The composition of claim 45, wherein the immunoregulatory molecule comprises one or more of IL-2 and low-toxicity IL-2 and one or more of CCL19, CCL21, and LEC.

57. The composition of claim 45, wherein the immunoregulatory molecule comprises IL-15 and one or more of CCL19, CCL21, and LEC.

58. The composition of claim 45, wherein the immunoregulatory molecule comprises IL-21 and one or more of CCL19, CCL21, and LEC.

59. The composition of claim 45, wherein the immunoregulatory molecule comprises GM-CSF and one or more of CCL19, CCL21, and LEC.

60. The composition of claim 45, wherein the immunoregulatory molecule comprises OX40L and one or more of CCL19, CCL21, and LEC.

61. The composition of claim 45, wherein the immunoregulatory molecule comprises CD137L and one or more of CCL19, CCL21, and LEC.

62. The composition of claim 45, wherein the immunoregulatory molecule comprises B7.1 and one or more of CCL19, CCL21, and LEC.

63. The composition of claim 45, wherein the immunoregulatory molecule comprises CD40L and one or more of CCL19, CCL21, and LEC.

64. The composition of claim 45, wherein the immunoregulatory molecule comprises GITRL and one or more of CCL19, CCL21, and LEC.

65. An isolated complex comprising an isolated cell comprising the CAR of claim 1 bound to a cell expressing a TNT antigen.

66. An isolated complex comprising the isolated cell comprising the CAR of any one of claim 1 bound to a TNT relevant antigen or a fragment thereof.

67. A method of producing a TNT CAR expressing cell comprising transducing an isolated cell with the isolated nucleic acid sequence of claim 12.

68. The method of claim 67, further comprising:
transducing the cell an isolated nucleic acid comprising an NFAT regulatory polynucleotide operatively linked to a polynucleotide encoding an immunoregulatory molecule.

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. A method of inhibiting the growth of a tumor expressing a TNT relevant antigen, comprising contacting the tumor with the isolated cell of claim 41.

91. A method of claim 90, wherein the contacting is in vitro or in vivo.

92. The method of claim 91, wherein the contacting is in vivo and the isolated cells are autologous to a subject being treated.

93. The method of claim 92, further comprising administering to the subject an effective amount of a cytoreductive therapy.

94. The method of claim 93, wherein the cytoreductive therapy comprises chemotherapy, cryotherapy, and/or radiation therapy.

95. A method of treating cancer expressing a TNT relevant antigen in a subject in need thereof, comprising administering to the subject an effective amount of the isolated cell of claim 41.

96. The method of claim 95, wherein the isolated cells are autologous to the subject being treated.

97. The method of claim 95, further comprising administering to the subject an effective amount of a cytoreductive therapy.

98. The method of claim 97, wherein the cytoreductive therapy comprises chemotherapy, cryotherapy, and/or radiation therapy.

99. The method of claim 95, wherein the cancer is a cancer that affects the blood and/or the bone marrow.

100. The method of claim 95, wherein the subject is a human patient.

101. A kit comprising the composition of claim 1, instructions for use.

102. (canceled)
